(19) United States
(12) Patent Application Publication
(10) Pub. No.: US 2023/0265501 A1
(43) Pub. Date: Aug. 24, 2023

(54) PHASE PROTECTIVE REAGENT FLOW ORDERING

(71) Applicant: Singular Genomics Systems, Inc., San Diego, CA (US)

(72) Inventors: Timothy LOONEY, Austin, TX (US); Eli N. GLEZER, Del Mar, CA (US); Yuji ISHITSUKA, San Diego, CA (US)

(21) Appl. No.: 18/069,078

(22) Filed: Dec. 20, 2022

Related U.S. Application Data
(63) Continuation of application No. PCT/US2022/020776, filed on Mar. 17, 2022.
(60) Provisional application No. 63/229,252, filed on Aug. 4, 2021.
(60) Provisional application No. 63/162,383, filed on Mar. 17, 2021.

(51) Int. Cl. C12Q 1/6869

(52) U.S. Cl. CPC C12Q 1/6869

(57) ABSTRACT

Disclosed herein, inter alia, are phase protective reagent flow orders and methods useful for improving sequencing efficiency.

### CROSS-REFERENCES TO RELATED APPLICATIONS

This application claims the benefit of U.S. Provisional Application No. 63/162,383, filed Mar. 17, 2021, and U.S. Provisional Application No. 63/229,252, filed Aug. 4, 2021, each of which are incorporated herein by reference in their entirety and for all purposes.

### BACKGROUND

Traditional sequencing-by-synthesis (SBS) methodologies employ serial incorporation and detection of labeled nucleotide analogues. For example, high-throughput SBS technology uses cleavable fluorescent nucleotide reversible terminator (NRT) sequencing chemistry. These cleavable fluorescent NRTs were designed based on the following rationale: each of the four nucleotide types (dA, dC, dG, dT, and/or dU) is modified by attaching a unique cleavable fluorophore to the specific location of the nucleobase and capping the 3′—OH group of the nucleotide sugar with a small reversible moiety (also referred to herein as a reversible terminator) so that they are still recognized by DNA polymerase as substrates. The reversible terminator temporarily halts the polymerase reaction after nucleotide incorporation while the fluorophore signal is detected. After incorporation and signal detection, the fluorophore and the reversible terminator are cleaved to resume the polymerase reaction in the next cycle. Typically, many polynucleotides are confined to an area of a discrete region (referred to as a cluster) and are synchronized in their nucleotide incorporation and detection. Some strands may extend faster or slower than their surrounding counterparts, resulting in the clusters of monoclonal amplicons being out-of-phase. During SBS, dephasing leads to signal loss and lowered base call accuracy, ultimately restricting the maximum read length produced by a sequencing device.

### BRIEF SUMMARY

In view of the foregoing, innovative approaches to address issues with existing sequencing technologies are needed. To increase sequencing efficiency, accuracy, and permit longer sequencing read lengths, there is a need for new strategies to correct dephasing. Disclosed herein, inter alia, are solutions to these and other problems in the art which, in embodiments, increase the fidelity and accuracy of high throughput sequencing methods. In certain embodiments, the compositions and methods provided herein reduce the amount of dephasing by traditional next generation sequencing techniques. In embodiments, the phase protective flow orders described herein reduce signal loss and improve base call accuracy compared to traditional next generation sequencing methods.

In an aspect is provided a method for sequencing a nucleic acid template, said method including: a) hybridizing one or more sequencing primers to the nucleic acid template; b) executing a plurality of sequencing cycles, each cycle including: (i) contacting the nucleic acid template with a sequencing solution in the presence of a polymerase; and (ii) detecting a characteristic signature indicating that the nucleotide has been incorporated into the sequencing primer; wherein the sequencing solutions of at least two sequencing cycles include a different combination of fewer than four nucleotide types, wherein each nucleotide of each nucleotide type includes a reversible terminator.

In an aspect is provided a method for extending a primer hybridized to a nucleic acid template, the method including: (a) contacting the primer with a first extension solution in the presence of a polymerase; (b) contacting the primer with a second extension solution in the presence of a polymerase thereby extending the primer by a single nucleotide; wherein: the first extension solution includes a first doublet of nucleotide types and the second extension solution includes a second doublet of nucleotide types, wherein the first doublet of nucleotide types have no nucleotide types in common with the second doublet of nucleotide types; and (c) repeating steps (a) and (b), wherein each repetition of steps (a) and (b) is a cycle, wherein each cycle is performed at least 20 times thereby performing a series of cycles, wherein each cycle is a first ordered cycle or a second ordered cycle, wherein the first ordered cycle contacts the primer with the first extension solution first and the second extension solution second, wherein the second ordered cycle contacts the primer with the second extension solution first and the first extension solution second, wherein the series of cycles is performed according to a non-cyclic sequence.

In an aspect is provided a method for extending a primer hybridized to a nucleic acid template, the method including: (a) contacting the primer with a first extension solution in the presence of a polymerase; (b) contacting the primer with a second extension solution in the presence of a polymerase thereby extending the primer by a single nucleotide; wherein: the first extension solution includes a first triplet of nucleotide types and the second extension solution includes a second triplet of nucleotide types, wherein the first triplet of nucleotide types has one or two nucleotide types in common with the second triplet of nucleotide types; and (c) repeating steps (a) and (b), wherein each repetition of steps (a) and (b) is a cycle, wherein each cycle is performed at least 20 times thereby performing a series of cycles, wherein each cycle is a first ordered cycle or a second ordered cycle, wherein the first ordered cycle contacts the primer with the first extension solution first and the second extension solution second, wherein the second ordered cycle contacts the primer with the second extension solution first and the first extension solution second, wherein the series of cycles is performed according to a non-cyclic sequence.

In an aspect is provided a method for extending a primer hybridized to a nucleic acid template, the method including: (a) contacting the primer with a first extension solution in the presence of a polymerase; (b) contacting the primer with a second extension solution in the presence of a polymerase thereby extending the primer by a single nucleotide; wherein: the first extension solution includes a first doublet of nucleotide types and the second extension solution includes a second triplet of nucleotide types, wherein the first doublet of nucleotide types has one or two nucleotide types in common with the second triplet of nucleotide types; and (c) repeating steps (a) and (b), wherein each repetition of steps (a) and (b) is a cycle, wherein each cycle is performed at least 20 times thereby performing a series of cycles, wherein each cycle is a first ordered cycle or a second ordered cycle, wherein the first ordered cycle contacts the primer with the first extension solution first and the second extension solution second, wherein the second ordered cycle contacts the primer with the second extension solution first and the first extension solution second, wherein the series of cycles is performed according to a non-cyclic sequence.

In an aspect is provided a method for extending a primer hybridized to a nucleic acid template, the method including: (a) contacting the primer with a first extension solution in the presence of a polymerase; (b) contacting the primer with a second extension solution in the presence of a polymerase thereby extending the primer by a single nucleotide; wherein: the first extension solution includes a first triplet of nucleotide types and the second extension solution includes a second doublet of nucleotide types, wherein the first triplet of nucleotide types has one or two nucleotide types in common with the second doublet of nucleotide types; and (c) repeating steps (a) and (b), wherein each repetition of steps (a) and (b) is a cycle, wherein each cycle is performed at least 20 times thereby performing a series of cycles, wherein each cycle is a first ordered cycle or a second ordered cycle, wherein the first ordered cycle contacts the primer with the first extension solution first and the second extension solution second, wherein the second ordered cycle contacts the primer with the second extension solution first and the first extension solution second, wherein the series of cycles is performed according to a non-cyclic sequence.

In an aspect is provided a kit for determining the identity of a base in a target nucleic acid by sequencing-by-synthesis, the kit including (a) a first mixture of deoxyribonucleotide triphosphates (dNTPs) including: a first plurality of dNTPs including a first label; and a second plurality of dNTPs including a second label; and (b) a second mixture of deoxyribonucleotide triphosphates (dNTPs) including: a third plurality of dNTPs comprising the first label; and a fourth plurality of dNTPs including the second label; wherein each of the first, second, third, and fourth pluralities of dNTPs is selected from the group consisting of dATP, dTTP, dCTP, and dGTP, and are different from each other; and wherein the first label and the second label are different labels and are distinguishable.

In an aspect is provided a kit for determining the identity of a base in a target nucleic acid by sequencing-by-synthesis, the kit including (a) a first mixture of deoxyribonucleotide triphosphates (dNTPs) including: a first plurality of dNTPs including a first label; a second plurality of dNTPs including a second label; and a third plurality of dNTPs including a third label; (b) a second mixture of deoxyribonucleotide triphosphates (dNTPs) including: the second plurality of dNTPs including the first label; the third plurality of dNTPs including the second label; and a fourth plurality of dNTPs including the third label; (c) a third mixture of dNTPs including: the fourth plurality of dNTPs including the first label; the first plurality of dNTPs including the second label; and the second plurality of dNTPs including the third label; and (d) a fourth mixture of dNTPs including: the third plurality of dNTPs including the first label; the fourth plurality of dNTPs including the second label; and the first plurality of dNTPs including the third label; wherein each of the first, second, third, and fourth pluralities of dNTPs is selected from the group consisting of dATP, dTTP, dCTP, and dGTP, and are different from each other; and wherein the first label, the second label, and the third label are different labels and are distinguishable.

In an aspect is provided a kit for determining the identity of a base in a target nucleic acid by sequencing-by-synthesis, the kit including (a) a first mixture of deoxyribonucleotide triphosphates (dNTPs) including: a first plurality of dNTPs including a first label; and a second plurality of dNTPs including a second label; and (b) a second mixture of deoxyribonucleotide triphosphates (dNTPs) including: a third plurality of dNTPs including the first label; and a fourth plurality of dNTPs including the second label; and (c) a third mixture including non-incorporating dNTPs including: a first plurality of non-incorporating dNTPs; a second plurality of non-incorporating dNTPs; a third plurality of non-incorporating dNTPs; and a fourth plurality of non-incorporating dNTPs; wherein each of the first, second, third, and fourth pluralities of dNTPs is selected from the group consisting of dATP, dTTP, dCTP, and dGTP, and are different from each other; wherein the first label and the second label are different labels and are distinguishable; and wherein each of the first, second, third, and fourth pluralities of non-incorporating dNTPs is selected from the group consisting of a non-incorporable analog of dATP, a non-incorporable analog of dTTP, a non-incorporable analog of dCTP, and a non-incorporable analog of dGTP, and are different from each other.

In an aspect is provided a device for sequencing a nucleic acid template, including: i) a reaction vessel for receiving flows of different solutions, wherein each of a plurality of the solutions includes a different combination of two to three nucleotide types, wherein each nucleotide of each nucleotide type includes a reversible terminator; ii) a plurality of reservoirs that each contain a different nucleotide type; iii) flow paths from each reservoir to the reaction vessel; and iv) a fluidics controller that controls the flow from the reservoirs to the reaction vessel, wherein the fluidics controller is programmed to randomly provide flow from the plurality of reservoirs to the reaction vessel in order to form the solutions.

In an aspect is provided a device for sequencing a nucleic acid template, including: i) a reaction vessel for receiving flows of different solutions, wherein each of a plurality of the solutions includes a different combination of two to three nucleotide types, wherein each nucleotide of each nucleotide type includes a reversible terminator; ii) a first reservoir that contains a first solution and a second reservoir that contains a second solution, wherein the first and second solutions collectively include all four nucleotide types; iii) flow paths from each reservoir to the reaction vessel; and iv) a fluidics controller that controls the flow from the reservoirs to the reaction vessel, wherein the fluidics controller is programmed to randomly provide flow from each of the reservoirs to the reaction vessel.

In an aspect is provided a device for sequencing a nucleic acid template, including: i) a reaction vessel for receiving flows of different solutions, wherein each of a plurality of the solutions includes a different combination of four nucleotide types, wherein at least one nucleotide type is a non-incorporating nucleotide type, and the remaining nucleotide types each include a reversible terminator; ii) a first reservoir that contains a first solution and a second reservoir that contains a second solution, wherein the first and second solutions collectively include all four nucleotide types; iii) flow paths from each reservoir to the reaction vessel; and iv) a fluidics controller that controls the flow from the reservoirs to the reaction vessel, wherein the fluidics controller is programmed to randomly provide flow from each of the reservoirs to the reaction vessel.

### BRIEF DESCRIPTION OF THE DRAWINGS

FIG. 1 illustrates a workflow for simulating dephasing during sequencing-by-synthesis. The simulation consists of 1000 cluster objects each composed of 1000 copies of a 1000 base template sequence composed of the four DNA bases in a random ordering. Cluster template sequences are read by successively exposing the clusters to up to four nucleotides as indicated by a given nucleotide flow order. For each of the 1000 template copies within a cluster, the simulation checks whether the next nucleotide matches one of the nucleotides present in the cycle and whether a lead error (incorporation of two bases) or lag error (failure to incorporate a base) has occurred, modeling each as a random process with 1% error probability. If an error does not occur, a single base is added to the extending template copy. After each successive cycle, the simulation determines the average fraction of template copies lacking lead or lag errors (in phase templates), as well as the average number of bases incorporated (i.e., read) across all clusters in the model.

FIG. 2 illustrates fraction templates in phase as a function of nucleotide flow order and cycle number. Dephasing during sequencing by synthesis was simulated as described in FIG. 1 for a four-nucleotide (default) flow order and alternative flow orders. Random AB consists of a random selection between two reagents, reagent A which contains nucleotides dA and dC; and reagent flow B which includes nucleotides dT and dG; de Bruijn B(2,5) indicates a selection between two two-nucleotide flows, where the ordering of the two solutions follows a de Bruijn sequence of order 5 with an alphabet of size 2; Random 3 consists of a random selection of three of the four nucleotides each flow (i.e., each flow contains 3 nucleotides); de Bruijn B(2,4) indicates a selection between two two-nucleotide flows, where the ordering of the two solutions follows a de Bruijn sequence of order 4 with an alphabet of size 2; Gafieira indicates the single nucleotide per flow order of the same name from U.S. Pat. Application Publication US2012/0264621, modified such that three consecutive bases of the order are delivered per cycle; Samba indicates the single nucleotide per flow order of the same name from U.S. Pat. Application Publication US2012/026462, modified such that three consecutive bases of the order are delivered per cycle; de Bruijn B(2,3) indicates a selection between two two-nucleotide flows, where the ordering of the two solutions follows a de Bruijn sequence of order 3 with an alphabet of size 2; Rotating AABB indicates alternating between two two-nucleotide flows A and B in a repeated ordering of AABB; Rotating AB indicates a regular alternation between two two-nucleotide flows A and B. In either AABB or AB, A may represent, for example, the two purine nucleotides, while B may represent, for example, the two pyrimidine nucleotides. The legend labels are sorted from top to bottom in descending order based on the fraction of in phase templates of each flow at a read length of 500bp.

FIGS. 3A-3B illustrate the performance of the Random AB and de Bruijn B(2,5) flow orders presented in FIG. 2, where the simulation has been extended to generate 1000 bp mean read lengths. FIG. 3A indicates the fraction of in phase templates per cluster at a mean read length of 1000 bp for Random AB and de Bruijn B(2,5) flow orders. For each of the 1000 clusters within each simulation, the fraction of in phase templates was determined, where in phase templates correspond to those having the mode number of base incorporations per cluster. At 1000 bp, the random flow order showed a higher overall fraction of in phase templates than the B(2,5) sequence derived flow order (mean 50.1% vs 41.2%), but also a higher variance across clusters (standard deviations of 0.138 and 0.849, respectively). FIG. 3B indicates the phasing profile at a mean read length of 1000 bp for Random AB and de Bruijn B(2,5) flow orders. For each of the 1000 extending template sequences within each cluster in the simulation, a phasing offset was obtained, measured as the number of the base pairs a template sequence was ahead or behind of the mode number of base incorporations for the cluster. Results indicate populations of synchronized out of phase molecules resulting from application of the de Bruijn B(2,5) sequence derived flow order. Synchronized populations of out of phase molecules are not evident in the simulation using the Random AB flow order.

FIG. 4 indicates results from the simulated performance of 888 permutations of a 12-length cyclic two solution flow order. That data presented in FIG. 4 indicate the relationship between sequencing efficiency, measured as the number of cycles required to produce a mean read length of 500bp, and the fraction of in phase templates at a mean read length of 500 bp. There is a strong correlation (r = 0.78), indicating a general tradeoff between sequencing efficiency and phase protection.

FIG. 5 illustrates the fraction of in phase templates as a function of nucleotide flow order and cycle number. Dephasing during sequencing by synthesis was simulated as described in FIG. 1 for a four-nucleotide (default) flow order and alternative flow orders. Random AB consists of a random selection between two reagents, reagent A which contains nucleotides dA and dC; and reagent flow B which includes nucleotides dT and dG; Thue-Morse, Thue-Morse 2, Thue-Morse 2b, Thue-Morse 3, and Thue-Morse 4 flow orders consist of a selection between two reagents as indicated by the flow order sequences in Table 2. Legend labels are sorted from top to bottom in descending order based on the fraction of in phase templates of each flow at a read length of 500 bp.

FIGS. 6A-6B illustrate a comparison of the performance of the Random AB flow order presented in FIGS. 2 and 3 with that of the Thue-Morse 2 sequence following simulation to generate 1000bp mean read lengths. FIG. 6A indicates the fraction of in phase templates per cluster at a mean read length of 1000bp for Random AB and Thue-Morse 2 flow orders. For each of the 1000 clusters within each simulation, the fraction of in phase templates was determined, where in phase templates correspond to those having the mode number of base incorporations per cluster. At 1000bp, the Random AB and Thue-Morse 2 flow orders showed a similar overall fraction of in phase templates (mean 50.1% vs 50.1%, respectively) and variance across clusters (standard deviations of .138 and .122, respectively). FIG. 6B indicates the phasing profile at a mean read length of 1000bp for Random AB and Thue-Morse 2 flow orders. For each of the 1000 extending template sequences within each cluster in the simulation a phasing offset was obtained, measured as the number of the base pairs a template sequence was ahead or behind of the mode number of base incorporations for the cluster.

FIG. 7 illustrates normalized channel signal intensity per cycle following sequencing via a four nucleotide (default) flow. Sequencing-by-synthesis was performed using four nucleotides each labeled by a separate fluorescent dye (C: green, T: yellow, G: orange, A: red). The net signal intensity deriving from each of the four dyes is presented over 55 flow cycles.

FIG. 8 illustrates normalized channel signal intensity per cycle following sequencing by synthesis via a three-nucleotide alternative flow order. 10 four nucleotides flows were followed by 70 flows consisting of a random selection of three of the four nucleotides (corresponding to “Random 3” flow in FIG. 1). Values are displayed for cycles 1-55 of the 70-cycle experiment. Cycles 10-55 demonstrate a drop in signal intensity for one of the four dyes each cycle, reflecting the absence of one of the four nucleotides from each flow cycle.

FIG. 9 illustrates normalized channel signal intensity per cycle following sequencing by synthesis via a three-nucleotide alternative flow order. 10 four nucleotides flows were followed by 70 flows consisting of a random selection of three of the four nucleotides (corresponding to “Random 3” flow in FIG. 1). Values are displayed for cycles 1-55 of the 70-cycle experiment. Cycles 10-55 demonstrate a drop in signal intensity for one of the four dyes each cycle, reflecting the absence of one of the four nucleotides from each flow cycle.

FIG. 10 illustrates estimated in phase templates per cycle for four nucleotide (default) and random three nucleotide flow orders. Sequencing-by-synthesis was performed using four nucleotide and three nucleotide flow orders as described in FIG. 8. For each of the approximately 8000 clusters analyzed per condition, the fraction of in phase template molecules (i.e., lacking a lag or lead error) is calculated as the signal deriving from the fluorescent channel corresponding to the expected base incorporation for a given cycle (incorporation matching the known template sequence) as a fraction of total signal for the cycle. Boxplots indicate the median.

FIG. 11 illustrates the variable (V), diversity (D), joining (J) and constant/isotype (C) region of the expressed, rearranged IGH receptor, including the membrane domain located at the 3′ end of the constant gene. Alternative splicing of membrane exons determines whether the translated receptor is membrane bound or secreted as an immunoglobulin. Alternating between two flow orders as part of interval sequencing allows one to determine the membrane exon and isotype, bypass the irrelevant body of the constant gene, then sequence the critical variable portion of the antibody while minimizing sequencing time.

FIG. 12 illustrates reconstruction of a genomic breakpoint region by consensus assembly of sequences produced by alternating between two flow orders as part of interval sequencing. Consensus assembly of the sequence fragments produces the full sequence of the region, precisely mapping the breakpoint junction.

FIGS. 13A-13B illustrate production of a higher fidelity consensus sequence. FIG. 13A illustrates production of a higher fidelity consensus sequence via long read sequencing of a DNA fragment containing tandem copies of a sequence of interest. Following sequencing, comparison of the sequence copies to one another enables detection and elimination of sequencing or PCR derived errors. FIG. 13B illustrates production of a higher fidelity consensus sequence by combining a plurality of reads derived from application of light and dark sequencing flows, in order to create a higher fidelity consensus sequence.

### DETAILED DESCRIPTION

### I. Definitions

The practice of the technology described herein will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Examples of such techniques are available in the literature. Methods, devices and materials similar or equivalent to those described herein can be used in the practice of embodiments of this invention.

All patents, patent applications, articles and publications mentioned herein, both supra and infra, are hereby expressly incorporated herein by reference in their entireties.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art. The following definitions are provided to facilitate understanding of certain terms used frequently herein and are not meant to limit the scope of the present disclosure.

As used herein, the singular terms “a”, “an”, and “the” include the plural reference unless the context clearly indicates otherwise. Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/- 10% of the specified value. In embodiments, about means the specified value.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein, the term “control” or “control experiment” is used in accordance with its plain and ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

As used herein, the term “complement” is used in accordance with its plain and ordinary meaning and refers to a nucleotide (e.g., RNA nucleotide or DNA nucleotide) or a sequence of nucleotides capable of base pairing with a complementary nucleotide or sequence of nucleotides. As described herein and commonly known in the art the complementary (matching) nucleotide of adenosine is thymidine in DNA, or alternatively in RNA the complementary (matching) nucleotide of adenosine is uracil, and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence. “Duplex” means at least two oligonucleotides and/or polynucleotides that are fully or partially complementary undergo Watson-Crick type base pairing among all or most of their nucleotides so that a stable complex is formed. Complementary single stranded nucleic acids and/or substantially complementary single stranded nucleic acids can hybridize to each other under hybridization conditions, thereby forming a nucleic acid that is partially or fully double stranded. When referring to a double-stranded polynucleotide including a first strand hybridized to a second strand, it is understood that each of the first strand and the second strand are independently single-stranded polynucleotides. All or a portion of a nucleic acid sequence may be substantially complementary to another nucleic acid sequence, in some embodiments. As referred to herein, “substantially complementary” refers to nucleotide sequences that can hybridize with each other under suitable hybridization conditions. Hybridization conditions can be altered to tolerate varying amounts of sequence mismatch within complementary nucleic acids that are substantially complementary. Substantially complementary portions of nucleic acids that can hybridize to each other can be 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more complementary to each other. In some embodiments substantially complementary portions of nucleic acids that can hybridize to each other are 100% complementary. Nucleic acids, or portions thereof, that are configured to hybridize to each other often comprise nucleic acid sequences that are substantially complementary to each other.

As described herein, the complementarity of sequences may be partial, in which only some of the nucleic acids match according to base pairing, or complete, where all the nucleic acids match according to base pairing. Thus, two sequences that are complementary to each other, may have a specified percentage of nucleotides that complement one another (e.g., about 60%, preferably 65%, 70%, 75%, 80%, 85%, 90%, 91%, 92%, 93%, 94%, 95%, 96%, 97%, 98%, 99%, or higher complementarity over a specified region). In embodiments, two sequences are complementary when they are completely complementary, having 100% complementarity. In embodiments, sequences in a pair of complementary sequences form portions of a single polynucleotide with non-base-pairing nucleotides (e.g., as in a hairpin structure, with or without an overhang) or portions of separate polynucleotides. In embodiments, one or both sequences in a pair of complementary sequences form portions of longer polynucleotides, which may or may not include additional regions of complementarity.

As used herein, the term “contacting” is used in accordance with its plain ordinary meaning and refers to the process of allowing at least two distinct species (e.g., chemical compounds including biomolecules or cells) to become sufficiently proximal to react, interact or physically touch. However, the resulting reaction product can be produced directly from a reaction between the added reagents or from an intermediate from one or more of the added reagents that can be produced in the reaction mixture. The term “contacting” may include allowing two species to react, interact, or physically touch, wherein the two species may be a compound, nucleic acid, a protein, or enzyme (e.g., a DNA polymerase).

As used herein, the term “nucleic acid” is used in accordance with its plain and ordinary meaning and refers to nucleotides (e.g., deoxyribonucleotides or ribonucleotides) and polymers thereof in either single-, double- or multiple-stranded form, or complements thereof. The terms “polynucleotide,” “oligonucleotide,” “oligo” or the like refer, in the usual and customary sense, to a sequence of nucleotides. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA with linear or circular framework. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences. A “nucleoside” is structurally similar to a nucleotide, but is missing the phosphate moieties. An example of a nucleoside analogue would be one in which the label is linked to the base and there is no phosphate group attached to the sugar molecule. As may be used herein, the terms “nucleic acid oligomer” and “oligonucleotide” are used interchangeably and are intended to include, but are not limited to, nucleic acids having a length of 200 nucleotides or less. In some embodiments, an oligonucleotide is a nucleic acid having a length of 2 to 200 nucleotides, 2 to 150 nucleotides, 5 to 150 nucleotides or 5 to 100 nucleotides. The terms “polynucleotide,” “oligonucleotide,” “oligo” or the like refer, in the usual and customary sense, to a linear sequence of nucleotides. Oligonucleotides are typically from about 5, 6, 7, 8, 9, 10, 12, 15, 25, 30, 40, 50 or more nucleotides in length, up to about 100 nucleotides in length. In some embodiments, an oligonucleotide is a primer configured for extension by a polymerase when the primer is annealed completely or partially to a complementary nucleic acid template. A primer is often a single stranded nucleic acid. In certain embodiments, a primer, or portion thereof, is substantially complementary to a portion of an adapter. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. In some embodiments, an oligonucleotide may be immobilized to a solid support.

The term “non-incorporating nucleotide(s)” or “non-hydrolyzable nucleotide(s),” as used herein, refers to a nucleotide analog capable of binding transiently to a polymerase in a template-dependent manner. For example, a non-incorporating nucleotide is not capable of forming a phosphodiester bond with another nucleotide in a polymerase-dependent reaction involving the release of pyrophosphate. The non-incorporating nucleotide can bind reversibly to the polymerase and may or may not have a structure similar to that of a native nucleotide which may include base, sugar, and phosphate moieties. The non-incorporating nucleotides can bind the polymerase/template complex in a template-dependent manner or can act as a universal mimetic and bind the polymerase/template complex in a non-template-dependent manner. The non-incorporating nucleotides can be a nucleotide mimetic of incorporable nucleotides, such as adenosine, guanosine, cytidine, thymidine or uridine nucleotides. The non-incorporating nucleotide includes any compound having a nucleotide structure, or a portion thereof, which can bind a polymerase. The non-incorporating nucleotide may be a dye-labeled nucleotide. In one embodiment, the non-incorporating nucleotide having multiple phosphate or phosphonate groups, the linkage between the phosphate or phosphonate groups can be non-hydrolyzable by the polymerase. The non-hydrolyzable linkages include, but are not limited to, amino, alkyl, methyl, and thio groups. Non-incorporating nucleotide tetraphosphate analogs having alpha-thio or alpha-boreno substitutions having been described (Rank, U.S. Published Pat. Application No. 2008/0108082; and Gelfand, U.S. Published Pat. Application No. 2008/0293071). The non-incorporating nucleotides can be alpha-phosphate modified nucleotides, alpha-beta nucleotide analogs, beta-phosphate modified nucleotides, beta-gamma nucleotide analogs, gamma-phosphate modified nucleotides, caged nucleotides, or dinucleotide analogs. Many examples of non-incorporating nucleotides are known (Rienitz A et al. Nucleic Acids Research. 1985; 13(15):5685-5695, which is incorporated herein by reference in its entirety), including commercially-available ones from Jena Bioscience (Jena, Germany). In embodiments, the non-incorporating nucleotide is an α,β-methylene-2′-deoxynucleoside 5′-triphosphate, as described in Liang et al. J. Med. Chem. 2008; 51(20):6460-70 and Upton TG et al. Org. Lett. 2009; 11(9):1883-1886, each of which is incorporated herein by reference in its entirety. Examples of α,β-methylene-2′-deoxynucleoside 5′-triphosphates include dCMP-C-PP (2′-Deoxycytidine-5′-[(α,β)-methyleno]triphosphate), dTMP-C-PP (2′-Deoxythymidine-5′-[(α,β)-methyleno]triphosphate), dGMP-C-PP (2′-Deoxyguanosine-5′-[(α,β)-methyleno]triphosphate), and dAMP-C-PP (2′-Deoxyadenosine-5′-[(α,β)-methyleno]triphosphate).

The term “universal base analog,” as used herein, refers to a nucleotide analog that is capable of forming a base pair to any of the four natural nucleotide bases (e.g., cytosine (C), guanine (G), adenine (A), or thymine (T)). Thus, any other base may be paired with a universal base analog in a double-stranded polynucleotide. Universal base analogs may be divided into hydrogen bonding bases and pi-stacking bases. Hydrogen bonding bases form hydrogen bonds with any of the natural nucleobases. The hydrogen bonds formed by hydrogen bonding bases are weaker than the hydrogen bonds between natural nucleobases. Pi-stacking bases are non-hydrogen bonding, hydrophobic, aromatic bases that stabilize duplex polynucleotides by stacking interactions. Examples of hydrogen bonding bases include, but are not limited to, hypoxanthine (inosine), 7-deazahypoxanthine, 2-azahypoxanthine, 2-hydroxypurine, purine, and 4-Amino-1H-pyrazolo [3,4-d]pyrimidine. In embodiments, universal base analogs included in the bases in a universal region of a universal template strand are hydrogen bonding bases. In embodiments, all universal base analogs included in the bases in the universal region are inosine or derivatives thereof. Examples of pi-stacking bases include, but are not limited to, nitroimidazole, indole, benzimidazole, 5-fluoroindole, 5-nitroindole, N-indol-5-yl-formamide, isoquinoline, and methylisoquinoline. Examples of universal bases are discussed in Berger et al., Universal Bases for Hybridization, Replication and Chain Termination, Nucleic Acids Research 2000, August 1, 28(15) pp. 2911-2914; David Loakes, The Applications of Universal DNA Base Analogs, 29(12) Nucleic Acids Research 2437 (2001); and Feng Liang et al., Universal base analogs and their applications in DNA sequencing technology, 3 RSC Advances 14910-14928 (2013).

The term “predetermined nucleotide sequence,” as used herein, refers to an a priori polynucleotide sequence. For example, a predetermined nucleotide sequence is known in advance of synthesis or any observation technique. In embodiments, the predetermined polynucleotide sequence may be manually specified by a human user or generated by a computer system. The predetermined polynucleotide sequences may include about 100-200 nucleotides. The predetermined polynucleotide sequences may encode digital data. The specific polynucleotide sequence of nucleotide bases (e.g., GCTAGACCT) may encode a bit sequence (e.g., 011010). Proof of concept systems and techniques for storing data in polynucleotides have been previously demonstrated. See Lee Organick et al., Random Access in Large-Scale DNA Data Storage, 36:3 Nat. Biotech. 243 (2018) and Christopher N. Takahashi et al., Demonstration of End-to-End Automation of DNA Data Storage, 9 Sci. Rep. 4998 (2019).

The term “primer,” as used herein, is defined to be one or more nucleic acid fragments that may specifically hybridize to a nucleic acid template, be bound by a polymerase, and be extended in a template-directed process for nucleic acid synthesis. A primer can be of any length depending on the particular technique it will be used for. For example, PCR primers are generally between 10 and 40 nucleotides in length. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. The length and complexity of the nucleic acid hybridizing to the nucleic acid template may vary. One of skill can adjust these factors to provide optimum hybridization and signal production for a given hybridization procedure, and to provide a desired resolution among different genes or genomic locations. The primer permits the addition of a nucleotide residue thereto, or oligonucleotide or polynucleotide synthesis therefrom, under suitable conditions known in the art. In an embodiment the primer is a DNA primer, i.e., a primer consisting of, or largely consisting of, deoxyribonucleotide residues. The primers are designed to have a sequence that is the complement of a region of template/target DNA to which the primer hybridizes. The addition of a nucleotide residue to the 3′ end of a primer by formation of a phosphodiester bond results in a DNA extension product. The addition of a nucleotide residue to the 3′ end of the DNA extension product by formation of a phosphodiester bond results in a further DNA extension product. In another embodiment the primer is an RNA primer. In embodiments, a primer is hybridized to a target polynucleotide. A “primer” comprises a sequence that is complementary to a polynucleotide template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis.

As used herein, the terms “solid support” and “substrate” and “solid surface” refers to discrete solid or semi-solid surfaces to which a plurality of primers may be attached. A solid support may encompass any type of solid, porous, or hollow sphere, ball, cylinder, or other similar configuration composed of plastic, ceramic, metal, or polymeric material (e.g., hydrogel) onto which a nucleic acid may be immobilized (e.g., covalently or non-covalently). Solid supports may be in the form of discrete particles, which alone does not imply or require any particular shape. The term “particle” means a small body made of a rigid or semi-rigid material. The body can have a shape characterized, for example, as a sphere, oval, microsphere, or other recognized particle shape whether having regular or irregular dimensions. As used herein, the term “discrete particles” refers to physically distinct particles having discernible boundaries. The term “particle” does not indicate any particular shape. The shapes and sizes of a collection of particles may be different or about the same (e.g., within a desired range of dimensions, or having a desired average or minimum dimension). A particle may be substantially spherical (e.g., microspheres) or have a non-spherical or irregular shape, such as cubic, cuboid, pyramidal, cylindrical, conical, oblong, or disc-shaped, and the like. In embodiments, the particle has the shape of a sphere, cylinder, spherocylinder, or ellipsoid. Discrete particles collected in a container and contacting one another will define a bulk volume containing the particles, and will typically leave some internal fraction of that bulk volume unoccupied by the particles, even when packed closely together. In embodiments, cores and/or core-shell particles are approximately spherical. As used herein the term “spherical” refers to structures which appear substantially or generally of spherical shape to the human eye, and does not require a sphere to a mathematical standard. In other words, “spherical” cores or particles are generally spheroidal in the sense of resembling or approximating to a sphere. In embodiments, the diameter of a spherical core or particle is substantially uniform, e.g., about the same at any point, but may contain imperfections, such as deviations of up to 1, 2, 3, 4, 5 or up to 10%. Because cores or particles may deviate from a perfect sphere, the term “diameter” refers to the longest dimension of a given core or particle. Likewise, polymer shells are not necessarily of perfect uniform thickness all around a given core. Thus, the term “thickness” in relation to a polymer structure (e.g., a shell polymer of a core-shell particle) refers to the average thickness of the polymer layer.

A solid support may further comprise a polymer or hydrogel on the surface to which the primers are attached (e.g., the primers are covalently attached to the polymer, wherein the polymer is in direct contact with the solid support). Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefin copolymers, polyimides etc.), nylon, ceramics, resins, Zeonor, silica or silica-based materials including silicon and modified silicon, carbon, metals, inorganic glasses, optical fiber bundles, photopatternable dry film resists, UV-cured adhesives and polymers. The solid supports for some embodiments have at least one surface located within a flow cell. The solid support, or regions thereof, can be substantially flat. The solid support can have surface features such as wells, pits, channels, ridges, raised regions, pegs, posts or the like. The term solid support is encompassing of a substrate (e.g., a flow cell) having a surface comprising a polymer coating covalently attached thereto. In embodiments, the solid support is a flow cell. The term “flow cell” as used herein refers to a chamber including a solid surface across which one or more fluid reagents can be flowed. Examples of flow cells and related fluidic systems and detection platforms that can be readily used in the methods of the present disclosure are described, for example, in Bentley et al., Nature 456:53-59 (2008). In certain embodiments a substrate comprises a surface (e.g., a surface of a flow cell, a surface of a tube, a surface of a chip, surface of a particle), for example a metal surface (e.g., steel, gold, silver, aluminum, silicon and copper). In some embodiments a substrate (e.g., a substrate surface) is coated and/or comprises functional groups and/or inert materials. In certain embodiments a substrate comprises a bead, a chip, a capillary, a plate, a membrane, a wafer (e.g., silicon wafers), a comb, or a pin for example. In some embodiments a substrate comprises a bead and/or a nanoparticle. A substrate can be made of a suitable material, non-limiting examples of which include a plastic or a suitable polymer (e.g., polycarbonate, poly(vinyl alcohol), poly(divinylbenzene), polystyrene, polyamide, polyester, polyvinylidene difluoride (PVDF), polyethylene, polyurethane, polypropylene, and the like), borosilicate, silica, nylon, Wang resin, Merrifield resin, metal (e.g., iron, a metal alloy, sepharose, agarose, polyacrylamide, dextran, cellulose and the like or combinations thereof. In some embodiments a substrate comprises a magnetic material (e.g., iron, nickel, cobalt, platinum, aluminum, and the like). In certain embodiments a substrate comprises a magnetic bead (e.g., DYNABEADS®, hematite, AMPure XP). Magnets can be used to purify and/or capture nucleic acids bound to certain substrates (e.g., substrates comprising a metal or magnetic material).

As used herein, the term “polymer” refers to macromolecules having one or more structurally unique repeating units. The repeating units are referred to as “monomers,” which are polymerized for the polymer. Typically, a polymer is formed by monomers linked in a chain-like structure. A polymer formed entirely from a single type of monomer is referred to as a “homopolymer.” A polymer formed from two or more unique repeating structural units may be referred to as a “copolymer.” A polymer may be linear or branched, and may be random, block, polymer brush, hyperbranched polymer, bottlebrush polymer, dendritic polymer, or polymer micelles. The term “polymer” includes homopolymers, copolymers, tripolymers, tetra polymers and other polymeric molecules made from monomeric subunits. Copolymers include alternating copolymers, periodic copolymers, statistical copolymers, random copolymers, block copolymers, linear copolymers and branched copolymers. The term “polymerizable monomer” is used in accordance with its meaning in the art of polymer chemistry and refers to a compound that may covalently bind chemically to other monomer molecules (such as other polymerizable monomers that are the same or different) to form a polymer. Polymers can be hydrophilic, hydrophobic, or amphiphilic, as known in the art. Thus, “hydrophilic polymers” are substantially miscible with water and include, but are not limited to, polyethylene glycol and the like. “Hydrophobic polymers” are substantially immiscible with water and include, but are not limited to, polyethylene, polypropylene, polybutadiene, polystyrene, polymers disclosed herein, and the like. “Amphiphilic polymers” have both hydrophilic and hydrophobic properties and are typically copolymers having hydrophilic segment(s) and hydrophobic segment(s). Polymers include homopolymers, random copolymers, and block copolymers, as known in the art. The term “homopolymer” refers, in the usual and customary sense, to a polymer having a single monomeric unit. The term “copolymer” refers to a polymer derived from two or more monomeric species. The term “random copolymer” refers to a polymer derived from two or more monomeric species with no preferred ordering of the monomeric species. The term “block copolymer” refers to polymers having two or homopolymer subunits linked by covalent bond. Thus, the term “hydrophobic homopolymer” refers to a homopolymer which is hydrophobic. The term “hydrophobic block copolymer” refers to two or more homopolymer subunits linked by covalent bonds and which is hydrophobic.

As used herein, the term “hydrogel” refers to a three-dimensional polymeric structure that is substantially insoluble in water, but which is capable of absorbing and retaining large quantities of water to form a substantially stable, often soft and pliable, structure. In embodiments, water can penetrate in between polymer chains of a polymer network, subsequently causing swelling and the formation of a hydrogel. In embodiments, hydrogels are super-absorbent (e.g., containing more than about 90% water) and can be comprised of natural or synthetic polymers.

The term “surface” is intended to mean an external part or external layer of a substrate. The surface can be in contact with another material such as a gas, liquid, gel, polymer, organic polymer, second surface of a similar or different material, metal, or coating. The surface, or regions thereof, can be substantially flat. The substrate and/or the surface can have surface features such as wells, pits, channels, ridges, raised regions, pegs, posts or the like.

As used herein, the terms “cluster” and “colony” are used interchangeably to refer to a discrete site on a solid support that includes a plurality of immobilized polynucleotides and a plurality of immobilized complementary polynucleotides. The term “clustered array” refers to an array formed from such clusters or colonies. In this context the term “array” is not to be understood as requiring an ordered arrangement of clusters. The term “array” is used in accordance with its ordinary meaning in the art, and refers to a population of different molecules that are attached to one or more solid-phase substrates such that the different molecules can be differentiated from each other according to their relative location. An array can include different molecules that are each located at different addressable features on a solid-phase substrate. The molecules of the array can be nucleic acid primers, nucleic acid probes, nucleic acid templates or nucleic acid enzymes such as polymerases or ligases. Arrays useful in the invention can have densities that ranges from about 2 different features to many millions, billions or higher. The density of an array can be from 2 to as many as a billion or more different features per square cm. For example an array can have at least about 100 features/cm², at least about 1,000 features/cm², at least about 10,000 features /cm², at least about 100,000 features /cm², at least about 10,000,000 features /cm², at least about 100,000,000 features /cm², at least about 1,000,000,000 features /cm², at least about 2,000,000,000 features /cm²or higher. In embodiments, the arrays have features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher.

Nucleic acids, including e.g., nucleic acids with a phosphorothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

In some embodiments, a nucleic acid comprises a capture nucleic acid. A capture nucleic acid refers to a nucleic acid that is attached to a substrate (e.g., covalently attached). In some embodiments, a capture nucleic acid comprises a primer. In some embodiments, a capture nucleic acid is a nucleic acid configured to specifically hybridize to a portion of one or more nucleic acid templates (e.g., a template of a library). In some embodiments a capture nucleic acid configured to specifically hybridize to a portion of one or more nucleic acid templates is substantially complementary to a suitable portion of a nucleic acid template, or an amplicon thereof. In some embodiments a capture nucleic acid is configured to specifically hybridize to a portion of an adapter, or a portion thereof. In some embodiments a capture nucleic acid, or portion thereof, is substantially complementary to a portion of an adapter, or a complement thereof. In embodiments, a capture nucleic acid is a probe oligonucleotide. Typically, a probe oligonucleotide is complementary to a target polynucleotide or portion thereof, and further comprises a label (such as a binding moiety) or is attached to a surface, such that hybridization to the probe oligonucleotide permits the selective isolation of probe-bound polynucleotides from unbound polynucleotides in a population. A probe oligonucleotide may or may not also be used as a primer.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amio acid on a protein or polypeptide through a covalent, non-covalent, or other interaction.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

As used herein, the term “template nucleic acid” refers to any polynucleotide molecule that may be bound by a polymerase and utilized as a template for nucleic acid synthesis. A template nucleic acid may be a target nucleic acid. In general, the term “target nucleic acid” refers to a nucleic acid molecule or polynucleotide in a starting population of nucleic acid molecules having a target sequence whose presence, amount, and/or nucleotide sequence, or changes in one or more of these, are desired to be determined. In general, the term “target sequence” refers to a nucleic acid sequence on a single strand of nucleic acid. The target sequence may be a portion of a gene, a regulatory sequence, genomic DNA, cDNA, RNA including mRNA, miRNA, rRNA, or others. The target sequence may be a target sequence from a sample or a secondary target such as a product of an amplification reaction. A target nucleic acid is not necessarily any single molecule or sequence. For example, a target nucleic acid may be any one of a plurality of target nucleic acids in a reaction, or all nucleic acids in a given reaction, depending on the reaction conditions. For example, in a nucleic acid amplification reaction with random primers, all polynucleotides in a reaction may be amplified. As a further example, a collection of targets may be simultaneously assayed using polynucleotide primers directed to a plurality of targets in a single reaction. As yet another example, all or a subset of polynucleotides in a sample may be modified by the addition of a primer-binding sequence (such as by the ligation of adapters containing the primer binding sequence), rendering each modified polynucleotide a target nucleic acid in a reaction with the corresponding primer polynucleotide(s). In the context of selective sequencing, “target nucleic acid(s)” refers to the subset of nucleic acid(s) to be sequenced from within a starting population of nucleic acids.

In embodiments, a target nucleic acid is a cell-free nucleic acid. In general, the terms “cell-free,” “circulating,” and “extracellular” as applied to nucleic acids (e.g. “cell-free DNA” (cfDNA) and “cell-free RNA” (cfRNA)) are used interchangeably to refer to nucleic acids present in a sample from a subject or portion thereof that can be isolated or otherwise manipulated without applying a lysis step to the sample as originally collected (e.g., as in extraction from cells or viruses). Cell-free nucleic acids are thus unencapsulated or “free” from the cells or viruses from which they originate, even before a sample of the subject is collected. Cell-free nucleic acids may be produced as a byproduct of cell death (e.g., apoptosis or necrosis) or cell shedding, releasing nucleic acids into surrounding body fluids or into circulation. Accordingly, cell-free nucleic acids may be isolated from a non-cellular fraction of blood (e.g., serum or plasma), from other bodily fluids (e.g., urine), or from non-cellular fractions of other types of samples.

As used herein, the terms “analogue” and “analog”, in reference to a chemical compound, refers to compound having a structure similar to that of another one, but differing from it in respect of one or more different atoms, functional groups, or substructures that are replaced with one or more other atoms, functional groups, or substructures. In the context of a nucleotide, a “nucleotide analog” and “modified nucleotide” refer to a compound that, like the nucleotide of which it is an analog, can be incorporated into a nucleic acid molecule (e.g., an extension product) by a suitable polymerase, for example, a DNA polymerase in the context of a nucleotide analogue. The terms also encompass nucleic acids containing known nucleotide analogs or modified backbone residues or linkages, which are synthetic, naturally occurring, or non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see, e.g., see Eckstein, OLIGONUCLEOTIDES AND ANALOGUES: A PRACTICAL APPROACH, Oxford University Press) as well as modifications to the nucleotide bases such as in 5-methyl cytidine or pseudouridine.; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA)), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

As used herein, a “native” nucleotide is used in accordance with its plain and ordinary meaning and refers to a naturally occurring nucleotide that does not include an exogenous label (e.g., a fluorescent dye, or other label) or chemical modification such as those that may characterize a nucleotide analog (e.g., a reversible terminating moiety). Examples of native nucleotides useful for carrying out procedures described herein include: dATP (2′-deoxyadenosine-5′-triphosphate); dGTP (2′-deoxyguanosine-5′-triphosphate); dCTP (2′-deoxycytidine-5′-triphosphate); dTTP (2′-deoxythymidine-5′-triphosphate); and dUTP (2′-deoxyuridine-5′-triphosphate). A “canonical” nucleotide is an unmodified nucleotide.

As used herein, the term “modified nucleotide” refers to a nucleotide modified in some manner. Typically, a nucleotide contains a single 5-carbon sugar moiety, a single nitrogenous base moiety and 1 to three phosphate moieties. In embodiments, a nucleotide can include a blocking moiety (alternatively referred to herein as a reversible terminator moiety) and/or a label moiety. A blocking moiety on a nucleotide prevents formation of a covalent bond between the 3′ hydroxyl moiety of the nucleotide and the 5′ phosphate of another nucleotide. A blocking moiety on a nucleotide can be reversible, whereby the blocking moiety can be removed or modified to allow the 3′ hydroxyl to form a covalent bond with the 5′ phosphate of another nucleotide. A blocking moiety can be effectively irreversible under particular conditions used in a method set forth herein. In embodiments, the blocking moiety is attached to the 3′ oxygen of the nucleotide and is independently —NH₂, —CN, —CH₃, C₂-C₆ allyl (e.g., —CH₂—CH═CH₂), methoxyalkyl (e.g., —CH₂—O—CH₃), or —CH₂N₃. In embodiments, the blocking moiety is attached to the 3′ oxygen of the nucleotide and is independently

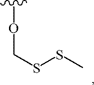

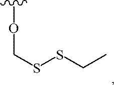

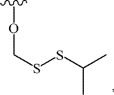

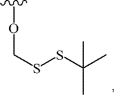

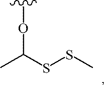

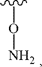

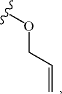

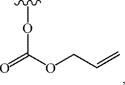

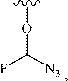

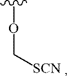

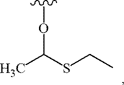

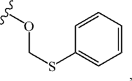

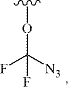

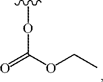

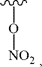

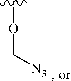

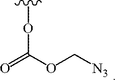

A label moiety of a nucleotide can be any moiety that allows the nucleotide to be detected, for example, using a spectroscopic method. Exemplary label moieties are fluorescent labels, mass labels, chemiluminescent labels, electrochemical labels, detectable labels and the like. One or more of the above moieties can be absent from a nucleotide used in the methods and compositions set forth herein. For example, a nucleotide can lack a label moiety or a blocking moiety or both. Examples of nucleotide analogues include, without limitation, 7-deaza-adenine, 7-deaza-guanine, the analogues of deoxynucleotides shown herein, analogues in which a label is attached through a cleavable linker to the 5-position of cytosine or thymine or to the 7-position of deaza-adenine or deaza-guanine, and analogues in which a small chemical moiety is used to cap the —OH group at the 3′-position of deoxyribose. Nucleotide analogues and DNA polymerase-based DNA sequencing are also described in U.S. Patent No. 6,664,079, which is incorporated herein by reference in its entirety for all purposes.

In embodiments, the nucleotides of the present disclosure use a cleavable linker to attach the label to the nucleotide. The use of a cleavable linker ensures that the label can, if required, be removed after detection, avoiding any interfering signal with any labelled nucleotide incorporated subsequently. The use of the term “cleavable linker” is not meant to imply that the whole linker is required to be removed from the nucleotide base. The cleavage site can be located at a position on the linker that ensures that part of the linker remains attached to the nucleotide base after cleavage. The linker can be attached at any position on the nucleotide base provided that Watson-Crick base pairing can still be carried out. In the context of purine bases, it is preferred if the linker is attached via the 7-position of the purine or the preferred deazapurine analogue, via an 8-modified purine, via an N-6 modified adenosine or an N-2 modified guanine. For pyrimidines, attachment is preferably via the 5-position on cytidine, thymidine or uracil and the N-4 position on cytosine.

The term “cleavable linker” or “cleavable moiety” as used herein refers to a divalent or monovalent, respectively, moiety which is capable of being separated (e.g., detached, split, disconnected, hydrolyzed, a stable bond within the moiety is broken) into distinct entities. A cleavable linker is cleavable (e.g., specifically cleavable) in response to external stimuli (e.g., enzymes, nucleophilic/basic reagents, reducing agents, photo-irradiation, electrophilic/acidic reagents, organometallic and metal reagents, or oxidizing reagents). A chemically cleavable linker refers to a linker which is capable of being split in response to the presence of a chemical (e.g., acid, base, oxidizing agent, reducing agent, Pd(0), tris-(2-carboxyethyl)phosphine, dilute nitrous acid, fluoride, tris(3-hydroxypropyl)phosphine), sodium dithionite (Na₂S₂O₄), or hydrazine (N₂H₄)). A chemically cleavable linker is non-enzymatically cleavable. In embodiments, the cleavable linker is cleaved by contacting the cleavable linker with a cleaving agent. In embodiments, the cleaving agent is a phosphine containing reagent (e.g., TCEP or THPP), sodium dithionite (Na₂S₂O₄), weak acid, hydrazine (N₂H₄), Pd(0), or light-irradiation (e.g., ultraviolet radiation). A chemically cleavable linker is non-enzymatically cleavable. In embodiments, the cleavable linker is cleaved by contacting the cleavable linker with a cleaving agent. In embodiments, the cleaving agent is a phosphine containing reagent (e.g., TCEP or THPP), sodium dithionite (Na₂S₂O₄), weak acid, hydrazine (N₂H₄), Pd(0), or light-irradiation (e.g., ultraviolet radiation). In embodiments, cleaving includes removing. A “cleavable site” or “scissile linkage” in the context of a polynucleotide is a site which allows controlled cleavage of the polynucleotide strand (e.g., the linker, the primer, or the polynucleotide) by chemical, enzymatic, or photochemical means known in the art and described herein. A scissile site may refer to the linkage of a nucleotide between two other nucleotides in a nucleotide strand (i.e., an intemucleosidic linkage). In embodiments, the scissile linkage can be located at any position within the one or more nucleic acid molecules, including at or near a terminal end (e.g., the 3′ end of an oligonucleotide) or in an interior portion of the one or more nucleic acid molecules. In embodiments, conditions suitable for separating a scissile linkage include a modulating the pH and/or the temperature. In embodiments, a scissile site can include at least one acid-labile linkage. For example, an acid-labile linkage may include a phosphoramidate linkage. In embodiments, a phosphoramidate linkage can be hydrolysable under acidic conditions, including mild acidic conditions such as trifluoroacetic acid and a suitable temperature (e.g., 30° C.), or other conditions known in the art, for example Matthias Mag, et al Tetrahedron Letters, Volume 33, Issue 48, 1992, 7319-7322. In embodiments, the scissile site can include at least one photolabile intemucleosidic linkage (e.g., o-nitrobenzyl linkages, as described in Walker et al, J. Am. Chem. Soc. 1988, 110, 21, 7170-7177), such as o-nitrobenzyloxymethyl or p-nitrobenzyloxymethyl group(s). In embodiments, the scissile site includes at least one uracil nucleobase. In embodiments, a uracil nucleobase can be cleaved with a uracil DNA glycosylase (UDG) or Formamidopyrimidine DNA Glycosylase Fpg. In embodiments, the scissile linkage site includes a sequence-specific nicking site having a nucleotide sequence that is recognized and nicked by a nicking endonuclease enzyme or a uracil DNA glycosylase.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site blast.ncbi.nlm.nih.gov/Blast.cgi or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

As used herein, the term “removable” group, e.g., a label or a blocking group or protecting group, is used in accordance with its plain and ordinary meaning and refers to a chemical group that can be removed from a nucleotide analogue such that a DNA polymerase can extend the nucleic acid (e.g., a primer or extension product) by the incorporation of at least one additional nucleotide. Removal may be by any suitable method, including enzymatic, chemical, or photolytic cleavage. Removal of a removable group, e.g., a blocking group, does not require that the entire removable group be removed, only that a sufficient portion of it be removed such that a DNA polymerase can extend a nucleic acid by incorporation of at least one additional nucleotide using a nucleotide or nucleotide analogue. In general, the conditions under which a removable group is removed are compatible with a process employing the removable group (e.g., an amplification process or sequencing process).

As used herein, the terms “blocking moiety,” “reversible blocking group,” “reversible terminator” and “reversible terminator moiety” are used in accordance with their plain and ordinary meanings and refer to a cleavable moiety which does not interfere with incorporation of a nucleotide comprising it by a polymerase (e.g., a DNA polymerase, such as a modified DNA polymerase), but prevents further strand extension until removed (“unblocked”). For example, a reversible terminator may refer to a blocking moiety located, for example, at the 3′ position of the nucleotide and may be a chemically cleavable moiety such as an allyl Group, an azidomethyl group or a methoxymethyl group, or may be an enzymatically cleavable group such as a phosphate ester. Suitable nucleotide blocking moieties are described in applications WO 2004/018497, U.S. Pat. Nos. 7,057,026, 7,541,444, WO 96/07669, U.S. Pat. Nos. 5,763,594, 5,808,045, 5,872,244 and 6,232,465 the contents of which are incorporated herein by reference in their entirety. The nucleotides may be labelled or unlabeled. The nucleotides may be modified with reversible terminators useful in methods provided herein and may be 3′—O—blocked reversible or 3′-unblocked reversible terminators. In nucleotides with 3′—O—blocked reversible terminators, the blocking group may be represented as -OR [reversible terminating (capping) group], wherein O is the oxygen atom of the 3′—OH of the pentose and R is the blocking group, while the label is linked to the base, which acts as a reporter and can be cleaved. The 3′—O—blocked reversible terminators are known in the art, and may be, for instance, a 3′-ONH₂reversible terminator, a 3′—O—allyl reversible terminator, or a 3′—O—azidomethyl reversible terminator. In embodiments, the reversible terminator moiety is

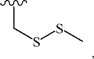

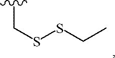

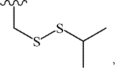

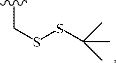

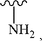

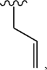

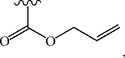

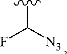

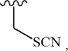

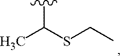

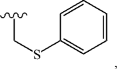

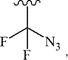

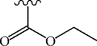

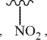

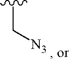

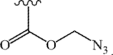

The term “allyl” as described herein refers to an unsubstituted methylene attached to a vinyl group (i.e., -CH=CH₂), having the formula

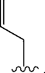

In embodiments, the reversible terminator moiety is

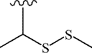

as described in US10,738,072, which is incorporated herein by reference for all purposes. For example, a nucleotide including a reversible terminator moiety may be represented by the formula:

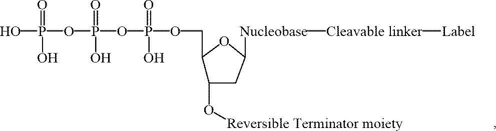

where the nucleobase is adenine or adenine analogue, thymine or thymine analogue, guanine or guanine analogue, or cytosine or cytosine analogue.

In some embodiments, a nucleic acid comprises a molecular identifier or a molecular barcode. As used herein, the term “molecular barcode” (which may be referred to as a “tag”, a “barcode”, a “molecular identifier”, an “identifier sequence” or a “unique molecular identifier” (UMI)) refers to any material (e.g., a nucleotide sequence, a nucleic acid molecule feature) that is capable of distinguishing an individual molecule in a large heterogeneous population of molecules. In embodiments, a barcode is unique in a pool of barcodes that differ from one another in sequence, or is uniquely associated with a particular sample polynucleotide in a pool of sample polynucleotides. In embodiments, every barcode in a pool of adapters is unique, such that sequencing reads comprising the barcode can be identified as originating from a single sample polynucleotide molecule on the basis of the barcode alone. In other embodiments, individual barcode sequences may be used more than once, but adapters comprising the duplicate barcodes are associated with different sequences and/or in different combinations of barcoded adapters, such that sequence reads may still be uniquely distinguished as originating from a single sample polynucleotide molecule on the basis of a barcode and adjacent sequence information (e.g., sample polynucleotide sequence, and/or one or more adjacent barcodes). In embodiments, barcodes are about or at least about 5, 6, 7, 8, 9, 10, 15, 20, 25, 30, 40, 50, 75 or more nucleotides in length. In embodiments, barcodes are shorter than 20, 15, 10, 9, 8, 7, 6, or 5 nucleotides in length. In embodiments, barcodes are about 10 to about 50 nucleotides in length, such as about 15 to about 40 or about 20 to about 30 nucleotides in length. In a pool of different barcodes, barcodes may have the same or different lengths. In general, barcodes are of sufficient length and include sequences that are sufficiently different to allow the identification of sequencing reads that originate from the same sample polynucleotide molecule. In embodiments, each barcode in a plurality of barcodes differs from every other barcode in the plurality by at least three nucleotide positions, such as at least 3, 4, 5, 6, 7, 8, 9, 10, or more nucleotide positions. In some embodiments, substantially degenerate barcodes may be known as random.

As used herein, the terms “label” and “labels” are used in accordance with their plain and ordinary meanings and refer to molecules that can directly or indirectly produce or result in a detectable signal either by themselves or upon interaction with another molecule. Non-limiting examples of detectable labels include fluorescent dyes, biotin, digoxin, haptens, and epitopes. In general, a dye is a molecule, compound, or substance that can provide an optically detectable signal, such as a colorimetric, luminescent, bioluminescent, chemiluminescent, phosphorescent, or fluorescent signal. In embodiments, the label is a dye. In embodiments, the dye is a fluorescent dye. Non-limiting examples of dyes, some of which are commercially available, include CF dyes (Biotium, Inc.), Alexa Fluor dyes (Thermo Fisher), DyLight dyes (Thermo Fisher), Cy dyes (GE Healthscience), IRDyes (Li-Cor Biosciences, Inc.), and HiLyte dyes (Anaspec, Inc.). In embodiments, a particular nucleotide type is associated with a particular label, such that identifying the label identifies the nucleotide with which it is associated. In embodiments, the label is luciferin that reacts with luciferase to produce a detectable signal in response to one or more bases being incorporated into an elongated complementary strand, such as in pyrosequencing. In embodiment, a nucleotide comprises a label (such as a dye). In embodiments, the label is not associated with any particular nucleotide, but detection of the label identifies whether one or more nucleotides having a known identity were added during an extension step (such as in the case of pyrosequencing).

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di-and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, homologs and isomers thereof, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (-O-). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

In embodiments, the detectable label is a fluorescent dye. In embodiments, the detectable label is a fluorescent dye capable of exchanging energy with another fluorescent dye (e.g., fluorescence resonance energy transfer (FRET) chromophores). Examples of detectable agents include imaging agents, including fluorescent and luminescent substances, including, but not limited to, a variety of organic or inorganic small molecules commonly referred to as “dyes,” “labels,” or “indicators.” Examples include fluorescein, rhodamine, acridine dyes, Alexa dyes, and cyanine dyes. In embodiments, the detectable moiety is a fluorescent molecule (e.g., acridine dye, cyanine, dye, fluorine dye, oxazine dye, phenanthridine dye, or rhodamine dye). In embodiments, the detectable moiety is a fluorescent molecule (e.g., acridine dye, cyanine, dye, fluorine dye, oxazine dye, phenanthridine dye, or rhodamine dye).

In embodiments, the detectable moiety is a moiety of a derivative of one of the detectable moieties described immediately above, wherein the derivative differs from one of the detectable moieties immediately above by a modification resulting from the conjugation of the detectable moiety to a compound described herein.

The term “cyanine” or “cyanine moiety” as described herein refers to a detectable moiety containing two nitrogen groups separated by a polymethine chain. In embodiments, the cyanine moiety has 3 methine structures (i.e., cyanine 3 or Cy3). In embodiments, the cyanine moiety has 5 methine structures (i.e., cyanine 5 or Cy5). In embodiments, the cyanine moiety has 7 methine structures (i.e., cyanine 7 or Cy7).

As used herein, the term “DNA polymerase” and “nucleic acid polymerase” are used in accordance with their plain ordinary meanings and refer to enzymes capable of synthesizing nucleic acid molecules from nucleotides (e.g., deoxyribonucleotides). Typically, a DNA polymerase adds nucleotides to the 3′- end of a DNA strand, one nucleotide at a time. In embodiments, the DNA polymerase is a Pol I DNA polymerase, Pol II DNA polymerase, Pol III DNA polymerase, Pol IV DNA polymerase, Pol V DNA polymerase, Pol β DNA polymerase, Pol µ DNA polymerase, Pol λ DNA polymerase, Pol σ DNA polymerase, Pol α DNA polymerase, Pol δ DNA polymerase, Pol ε DNA polymerase, Pol η DNA polymerase, Pol ℩ DNA polymerase, Pol κ DNA polymerase, Pol ζ DNA polymerase, Pol γ DNA polymerase, Pol θ DNA polymerase, Pol υ DNA polymerase, or a thermophilic nucleic acid polymerase (e.g. Therminator γ, 9°N polymerase (exo-), Therminator II, Therminator III, or Therminator IX). In embodiments, the DNA polymerase is a modified archaeal DNA polymerase. In embodiments, the polymerase is a reverse transcriptase. In embodiments, the polymerase is a mutant P. abyssi polymerase (e.g., such as a mutant P. abyssi polymerase described in WO 2018/148723 or WO 2020/056044). In embodiments, the polymerase is an enzyme described in US 2021/0139884. For example, a polymerase catalyzes the addition of a next correct nucleotide to the 3′—OH group of the primer via a phosphodiester bond, thereby chemically incorporating the nucleotide into the primer. Optionally, the polymerase used in the provided methods is a processive polymerase. Optionally, the polymerase used in the provided methods is a distributive polymerase.

As used herein, the term “thermophilic nucleic acid polymerase” refers to a family of DNA polymerases (e.g., 9°N™) and mutants thereof derived from the DNA polymerase originally isolated from the hyperthermophilic archaea, Thermococcus sp. 9 degrees N-7, found in hydrothermal vents at that latitude (East Pacific Rise) (Southworth MW, et al. PNAS. 1996;93(11):5281-5285). A thermophilic nucleic acid polymerase is a member of the family B DNA polymerases. Site-directed mutagenesis of the 3′-5′ exo motif I (Asp-Ile-Glu or DIE) to AIA, AIE, EIE, EID or DIA yielded polymerase with no detectable 3′ exonuclease activity. Mutation to Asp-Ile-Asp (DID) resulted in reduction of 3′-5′ exonuclease specific activity to <1% of wild type, while maintaining other properties of the polymerase including its high strand displacement activity. The sequence AIA (D141A, E143A) was chosen for reducing exonuclease. Subsequent mutagenesis of key amino acids results in an increased ability of the enzyme to incorporate dideoxynucleotides, ribonucleotides and acyclonucleotides (e.g., Therminator II enzyme from New England Biolabs with D141A / E143A / Y409V / A485L mutations); 3′-amino-dNTPs, 3′-azido-dNTPs and other 3′modified nucleotides (e.g., NEB Therminator III DNA Polymerase with D141A / E143A / L408S / Y409A / P410V mutations, NEB Therminator IX DNA polymerase), or γ-phosphate labeled nucleotides (e.g., Therminator γ: D141A / E143A / W355A / L408W / R460A / Q461S / K464E / D480V / R484W / A485L). Typically, these enzymes do not have 5′-3′ exonuclease activity. Additional information about thermophilic nucleic acid polymerases may be found in (Southworth MW, et al. PNAS. 1996;93(11):5281-5285; Bergen K, et al. ChemBioChem. 2013; 14(9):1058-1062; Kumar S, et al. Scientific Reports. 2012;2:684; Fuller CW, et al. 2016;113(19):5233-5238; Guo J, et al. Proceedings of the National Academy of Sciences of the United States of America. 2008;105(27):9145-9150), which are incorporated herein in their entirety for all purposes.

As used herein, the term “exonuclease activity” is used in accordance with its ordinary meaning in the art, and refers to the removal of a nucleotide from a nucleic acid by a DNA polymerase. For example, during polymerization, nucleotides are added to the 3′ end of the primer strand. Occasionally a DNA polymerase incorporates an incorrect nucleotide to the 3′—OH terminus of the primer strand, wherein the incorrect nucleotide cannot form a hydrogen bond to the corresponding base in the template strand. Such a nucleotide, added in error, is removed from the primer as a result of the 3′ to 5′ exonuclease activity of the DNA polymerase. In embodiments, exonuclease activity may be referred to as “proofreading.” When referring to 3′-5′ exonuclease activity, it is understood that the DNA polymerase facilitates a hydrolyzing reaction that breaks phosphodiester bonds at the 3′ end of a polynucleotide chain to excise the nucleotide. In embodiments, 3′-5′ exonuclease activity refers to the successive removal of nucleotides in single-stranded DNA in a 3′ → 5′ direction, releasing deoxyribonucleoside 5′-monophosphates one after another. Methods for quantifying exonuclease activity are known in the art, see for example Southworth et al, PNAS Vol 93, 8281-8285 (1996).

As used herein, the term “incorporating” or “chemically incorporating,” when used in reference to a primer and cognate nucleotide, refers to the process of joining the cognate nucleotide to the primer or extension product thereof by formation of a phosphodiester bond.

As used herein, the term “selective” or “selectivity” or the like of a compound refers to the compound’s ability to discriminate between molecular targets. When used in the context of sequencing, such as in “selectively sequencing,” this term refers to sequencing one or more target polynucleotides from an original starting population of polynucleotides, and not sequencing non-target polynucleotides from the starting population. Typically, selectively sequencing one or more target polynucleotides involves differentially manipulating the target polynucleotides based on known sequence. For example, target polynucleotides may be hybridized to a probe oligonucleotide that may be labeled (such as with a member of a binding pair) or bound to a surface. In embodiments, hybridizing a target polynucleotide to a probe oligonucleotide includes the step of displacing one strand of a double-stranded nucleic acid. Probe-hybridized target polynucleotides may then be separated from non-hybridized polynucleotides, such as by removing probe-bound polynucleotides from the starting population or by washing away polynucleotides that are not bound to a probe. The result is a selected subset of the starting population of polynucleotides, which is then subjected to sequencing, thereby selectively sequencing the one or more target polynucleotides.

As used herein, the terms “specific”, “specifically”, “specificity”, or the like of a compound refers to the compound’s ability to cause a particular action, such as binding, to a particular molecular target with minimal or no action to other proteins in the cell.

As used herein, the terms “bind” and “bound” are used in accordance with their plain and ordinary meanings and refer to an association between atoms or molecules. The association can be direct or indirect. For example, bound atoms or molecules may be directly bound to one another, e.g., by a covalent bond or non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). As a further example, two molecules may be bound indirectly to one another by way of direct binding to one or more intermediate molecules, thereby forming a complex.

As used herein, the terms “sequencing”, “sequence determination”, “determining a nucleotide sequence”, and the like include determination of partial as well as full sequence information, including the identification, ordering, or locations of the nucleotides that comprise the polynucleotide being sequenced, and inclusive of the physical processes for generating such sequence information. That is, the term includes sequence comparisons, fingerprinting, and like levels of information about a target polynucleotide, as well as the express identification and ordering of nucleotides in a target polynucleotide. The term also includes the determination of the identification, ordering, and locations of one, two, or three of the four types of nucleotides within a target polynucleotide. Sequencing methods, such as those outlined in U.S. Pat. No. 5,302,509 can be carried out using the nucleotides described herein. The sequencing methods are preferably carried out with the target polynucleotide arrayed on a solid substrate. Multiple target polynucleotides can be immobilized on the solid support through linker molecules, or can be attached to particles, e.g., microspheres, which can also be attached to a solid substrate. The solid substrate is in the form of a chip, a bead, a well, a capillary tube, a slide, a wafer, a filter, a fiber, a porous media, or a column. In some embodiments, the solid substrate is gold, quartz, silica, plastic, glass, diamond, silver, metal, or polypropylene. In some embodiments, the solid substrate is porous.

As used herein, the term “sequencing reaction mixture” is used in accordance with its plain and ordinary meaning and refers to an aqueous mixture that contains the reagents necessary to allow a nucleotide or nucleotide analogue to be added to a DNA strand by a DNA polymerase. In embodiments, the sequencing reaction mixture includes a buffer. In embodiments, the buffer includes an acetate buffer, 3-(N-morpholino) propanesulfonic acid (MOPS) buffer, N-(2-Acetamido)-2-aminoethanesulfonic acid (ACES) buffer, phosphate-buffered saline (PBS) buffer, 4-(2-hydroxyethyl)-1-piperazineethanesulfonic acid (HEPES) buffer, N-(1,1-Dimethyl-2-hydroxyethyl)-3-amino-2-hydroxypropanesulfonic acid (AMPSO) buffer, borate buffer (e.g., borate buffered saline, sodium borate buffer, boric acid buffer), 2-Amino-2-methyl-1,3-propanediol (AMPD) buffer, N-cyclohexyl-2-hydroxyl-3-aminopropanesulfonic acid (CAPSO) buffer, 2-Amino-2-methyl-1-propanol (AMP) buffer, 4-(Cyclohexylamino)-1-butanesulfonic acid (CABS) buffer, glycine-NaOH buffer, N-Cyclohexyl-2-aminoethanesulfonic acid (CHES) buffer, tris(hydroxymethyl)aminomethane (Tris) buffer, or aN-cyclohexyl-3-aminopropanesulfonic acid (CAPS) buffer. In embodiments, the buffer is a borate buffer. In embodiments, the buffer is a CHES buffer. In embodiments, the sequencing reaction mixture includes nucleotides, wherein the nucleotides include a reversible terminating moiety and a label covalently linked to the nucleotide via a cleavable linker. In embodiments, the sequencing reaction mixture includes a buffer, DNA polymerase, detergent (e.g., Triton X), a chelator (e.g., EDTA), or salts (e.g., ammonium sulfate, magnesium chloride, sodium chloride, or potassium chloride).

As used herein, the term “sequencing cycle” is used in accordance with its plain and ordinary meaning and refers to incorporating one or more nucleotides (e.g., nucleotide analogues) to the 3′ end of a polynucleotide with a polymerase, and detecting one or more labels that identify the one or more nucleotides incorporated. The sequencing may be accomplished by, for example, sequencing by synthesis, pyrosequencing, and the like. In embodiments, a sequencing cycle includes extending a complementary polynucleotide by incorporating a first nucleotide using a polymerase, wherein the complementary polynucleotide is hybridized to a template nucleic acid, detecting the first nucleotide, and identifying the first nucleotide. In embodiments, to begin a sequencing cycle, one or more differently labeled nucleotides and a DNA polymerase can be introduced. Following nucleotide addition, signals produced (e.g., via excitation and emission of a detectable label) can be detected to determine the identity of the incorporated nucleotide (based on the labels on the nucleotides). Reagents can then be added to remove the 3′ reversible terminator and to remove labels from each incorporated base. Reagents, enzymes and other substances can be removed between steps by washing. Cycles may include repeating these steps, and the sequence of each cluster is read over the multiple repetitions.

“Hybridize” shall mean the annealing of one single-stranded nucleic acid (such as a primer) to another nucleic acid based on the well-understood principle of sequence complementarity. In an embodiment the other nucleic acid is a single-stranded nucleic acid. The propensity for hybridization between nucleic acids depends on the temperature and ionic strength of their milieu, the length of the nucleic acids and the degree of complementarity. The effect of these parameters on hybridization is described in, for example, Sambrook J., Fritsch E. F., Maniatis T., Molecular cloning: a laboratory manual, Cold Spring Harbor Laboratory Press, New York (1989). As used herein, hybridization of a primer, or of a DNA extension product, respectively, is extendable by creation of a phosphodiester bond with an available nucleotide or nucleotide analogue capable of forming a phosphodiester bond, therewith. For example, hybridization can be performed at a temperature ranging from 15° C. to 95° C. In some embodiments, the hybridization is performed at a temperature of about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., or about 95° C. In other embodiments, the stringency of the hybridization can be further altered by the addition or removal of components of the buffered solution. In some embodiments nucleic acids, or portions thereof, that are configured to hybridize are often about 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more or 100% complementary to each other over a contiguous portion of nucleic acid sequence. A specific hybridization discriminates over non-specific hybridization interactions (e.g., two nucleic acids that are not configured to specifically hybridize, e.g., two nucleic acids that are 80% or less, 70% or less, 60% or less or 50% or less complementary) by about 2-fold or more, often about 10-fold or more, and sometimes about 100-fold or more, 1000-fold or more, 10,000-fold or more, 100,000-fold or more, or 1,000,000-fold or more. Two nucleic acid strands (e.g., two single-stranded polynucleotides) that are hybridized to each other can form a duplex which comprises a double-stranded portion of nucleic acid.

As used herein, the terms “dark cycle” and “limited-extension cycle” and “LE cycle” refer to incorporating with a polymerase one or more nucleotides (e.g., native nucleotides) to the 3′ end of a polynucleotide under a set of conditions that are different from a sequencing cycle. In embodiments, during a dark cycle the identity of a nucleotide is not determined following incorporation of the nucleotide. In embodiments, the identity of one or more (but not all) nucleotides is optionally determined upon incorporation. In embodiments, during a dark cycle, a native nucleotide (e.g., dATP, dCTP, dTTP, or dGTP) is incorporated into a polynucleotide. Due to it being a native nucleotide having no reversible terminator moiety, the polymerase does not temporarily halt, and the incorporated nucleotide is not detected or identified, and polymerization continues. In embodiments, during a dark cycle a nucleotide analogue comprising a label (e.g., dATP*, dCTP*, dTTP*, or dGTP*, wherein ‘*’ indicates a labeled nucleotide) may be used and is incorporated into a polynucleotide. The identity of the incorporated nucleotide may be determined to ensure cluster synchronization. The native nucleotides may be any number of naturally occurring or modified nucleotides. In embodiments, the nucleotides include a reversible blocking group (i.e., a reversible terminator moiety). In embodiments, a dark cycle includes the incorporation of one or more nucleotides that are unidentified, and optionally one or more nucleotides that are identified. Any number of native nucleotides may be incorporated into the dark-extension strand until a nucleotide analogue having a polymerase-compatible cleavable moiety (i.e., a reversible terminator moiety) is incorporated, which temporarily halts the polymerase reaction until the moiety is removed. Once the moiety is removed, another sequencing cycle or an additional dark cycle may be initiated. In embodiments, a series of dark cycles are performed before changing the reaction conditions to perform a series of sequencing cycles.

As used herein, the term “extension” or “elongation” is used in accordance with their plain and ordinary meanings and refer to synthesis by a polymerase of a new polynucleotide strand (i.e., an “extension strand”) complementary to a template strand by adding free nucleotides (e.g., dNTPs) from a reaction mixture that are complementary to the template in the 5′-to-3′ direction. Extension includes condensing the 5′-phosphate group of the dNTPs with the 3′-hydroxy group at the end of the nascent (elongating) DNA strand.

As used herein, the term “sequencing read” is used in accordance with its plain and ordinary meaning and refers to an inferred sequence of base pairs (or base pair probabilities) corresponding to all or part of a single DNA fragment. Sequencing technologies vary in the length of reads produced. A sequencing read may include 10, 20, 30, 40, 50, 60, 70, 80, 90, 100, 150, 200, 250, or more nucleotide bases. Reads of length 20-40 base pairs (bp) are referred to as ultra-short. Typical sequencers produce read lengths in the range of 100-500 bp. Read length is a factor which can affect the results of biological studies. For example, longer read lengths improve the resolution of de novo genome assembly and detection of structural variants. In some embodiments, a sequencing read may include 500, 600, 700, 800, 900, 1000, 1100, 1200, 1300, 1400, 1500, or more nucleotide bases. In embodiments, a sequencing read includes reading a barcode and a template nucleotide sequence. In embodiments, a sequencing read includes reading a template nucleotide sequence. In embodiments, a sequencing read includes reading a barcode and not a template nucleotide sequence. In embodiments, a sequencing read includes a computationally derived string corresponding to the detected label. The sequence reads are optionally stored in an appropriate data structure for further evaluation. In embodiments, a first sequencing reaction can generate a first sequencing read. The first sequencing read can provide the sequence of a first region of the polynucleotide fragment. In embodiments, a second sequencing primer can initiate sequencing at a second location on the nucleic acid template. The second location can be distinct from the first location. In some cases, a 3′ terminal nucleotide of the second primer can hybridize to a location that is more than 5 nucleotides away from a binding site of a 3′ terminal nucleotide of the first primer. The second sequencing reaction can generate a second sequencing read. The second sequencing read can provide the sequence of a second region of the nucleic acid template which is distinct from the first region of the nucleic acid template. In some embodiments, the nucleic acid template is optionally subjected to one or more additional rounds of sequencing using additional sequencing primers, thereby generating additional sequencing reads.

A nucleic acid can be amplified by a suitable method. The term “amplified” as used herein refers to subjecting a target nucleic acid in a sample to a process that linearly or exponentially generates amplicon nucleic acids having the same or substantially the same (e.g., substantially identical) nucleotide sequence as the target nucleic acid, or segment thereof, and/or a complement thereof. In some embodiments an amplification reaction comprises a suitable thermal stable polymerase. Thermal stable polymerases are known in the art and are stable for prolonged periods of time, at temperature greater than 80° C. when compared to common polymerases found in most mammals. In certain embodiments the term “amplified” refers to a method that comprises a polymerase chain reaction (PCR). Conditions conducive to amplification (i.e., amplification conditions) are known and often comprise at least a suitable polymerase, a suitable template, a suitable primer or set of primers, suitable nucleotides (e.g., dNTPs), a suitable buffer, and application of suitable annealing, hybridization and/or extension times and temperatures. In certain embodiments an amplified product (e.g., an amplicon) can contain one or more additional and/or different nucleotides than the template sequence, or portion thereof, from which the amplicon was generated (e.g., a primer can contain “extra” nucleotides (such as a 5′ portion that does not hybridize to the template), or one or more mismatched bases within a hybridizing portion of the primer).

A nucleic acid can be amplified by a thermocycling method or by an isothermal amplification method. In some embodiments, a rolling circle amplification method is used. In some embodiments, amplification takes place on a solid support (e.g., within a flow cell) where a nucleic acid, nucleic acid library or portion thereof is immobilized. In certain sequencing methods, a nucleic acid library is added to a flow cell and immobilized by hybridization to anchors under suitable conditions. This type of nucleic acid amplification is often referred to as solid phase amplification. In some embodiments of solid phase amplification, all or a portion of the amplified products are synthesized by an extension initiating from an immobilized primer. Solid phase amplification reactions are analogous to standard solution phase amplifications except that at least one of the amplification oligonucleotides (e.g., primers) is immobilized on a solid support.

In some embodiments solid phase amplification comprises a nucleic acid amplification reaction comprising only one species of oligonucleotide primer immobilized to a surface or substrate. In certain embodiments solid phase amplification comprises a plurality of different immobilized oligonucleotide primer species. In some embodiments solid phase amplification may comprise a nucleic acid amplification reaction comprising one species of oligonucleotide primer immobilized on a solid surface and a second different oligonucleotide primer species in solution. Multiple different species of immobilized or solution-based primers can be used. Non-limiting examples of solid phase nucleic acid amplification reactions include interfacial amplification, bridge amplification, emulsion PCR, WildFire amplification (e.g., US patent publication US20130012399), the like or combinations thereof.

Provided herein are methods and compositions for analyzing a sample (e.g., sequencing nucleic acids within a sample). A sample (e.g., a sample comprising nucleic acid) can be obtained from a suitable subject. A sample can be isolated or obtained directly from a subject or part thereof. In some embodiments, a sample is obtained indirectly from an individual or medical professional. A sample can be any specimen that is isolated or obtained from a subject or part thereof. A sample can be any specimen that is isolated or obtained from multiple subjects. Non-limiting examples of specimens include fluid or tissue from a subject, including, without limitation, blood or a blood product (e.g., serum, plasma, platelets, buffy coats, or the like), umbilical cord blood, chorionic villi, amniotic fluid, cerebrospinal fluid, spinal fluid, lavage fluid (e.g., lung, gastric, peritoneal, ductal, ear, arthroscopic), a biopsy sample, celocentesis sample, cells (blood cells, lymphocytes, placental cells, stem cells, bone marrow derived cells, embryo or fetal cells) or parts thereof (e.g., mitochondrial, nucleus, extracts, or the like), urine, feces, sputum, saliva, nasal mucous, prostate fluid, lavage, semen, lymphatic fluid, bile, tears, sweat, breast milk, breast fluid, the like or combinations thereof. A fluid or tissue sample from which nucleic acid is extracted may be acellular (e.g., cell-free). Non-limiting examples of tissues include organ tissues (e.g., liver, kidney, lung, thymus, adrenals, skin, bladder, reproductive organs, intestine, colon, spleen, brain, the like or parts thereof), epithelial tissue, hair, hair follicles, ducts, canals, bone, eye, nose, mouth, throat, ear, nails, the like, parts thereof or combinations thereof. A sample may comprise cells or tissues that are normal, healthy, diseased (e.g., infected), and/or cancerous (e.g., cancer cells). A sample obtained from a subject may comprise cells or cellular material (e.g., nucleic acids) of multiple organisms (e.g., virus nucleic acid, fetal nucleic acid, bacterial nucleic acid, parasite nucleic acid).

In some embodiments, a sample comprises nucleic acid, or fragments thereof. A sample can comprise nucleic acids obtained from one or more subjects. In some embodiments a sample comprises nucleic acid obtained from a single subject. In some embodiments, a sample comprises a mixture of nucleic acids. A mixture of nucleic acids can comprise two or more nucleic acid species having different nucleotide sequences, different fragment lengths, different origins (e.g., genomic origins, cell or tissue origins, subject origins, the like or combinations thereof), or combinations thereof. A sample may comprise synthetic nucleic acid.

A subject can be any living or non-living organism, including but not limited to a human, non-human animal, plant, bacterium, fungus, virus or protist. A subject may be any age (e.g., an embryo, a fetus, infant, child, adult). A subject can be of any sex (e.g., male, female, or combination thereof). A subject may be pregnant. In some embodiments, a subject is a mammal. In some embodiments, a subject is a human subject. A subject can be a patient (e.g., a human patient). In some embodiments a subject is suspected of having a genetic variation or a disease or condition associated with a genetic variation.

As used herein, the term “consensus sequence” refers to a sequence that shows the nucleotide most commonly found at each position within the nucleic acid sequences of group of sequences (e.g., a group of sequencing reads) aligned at that position. A consensus sequence is often “assembled” from shorter sequence reads that are at least partially overlapping. Where two sequences contain overlapping sequence information aligned at one end and non-overlapping sequence information at opposite ends, the consensus sequence formed from the two sequences will be longer than either sequence individually. Aligning multiple such sequences allows for assembly of many short sequences into much longer consensus sequences representative of a longer sample polynucleotide. In embodiments, aligned sequences used to generate a consensus sequence may contain gaps (e.g., representative of nucleotides not appearing in a given read because they were extended during a dark cycle and not identified).

Where a range of values is provided, it is understood that each intervening value, to the tenth of the unit of the lower limit unless the context clearly indicates otherwise, between the upper and lower limit of that range, and any other stated or unstated intervening value in, or smaller range of values within, that stated range is encompassed within the invention. The upper and lower limits of any such smaller range (within a more broadly recited range) may independently be included in the smaller ranges, or as particular values themselves, and are also encompassed within the invention, subject to any specifically excluded limit in the stated range. Where the stated range includes one or both of the limits, ranges excluding either or both of those included limits are also included in the invention.

The term “kit” is used in accordance with its plain ordinary meaning and refers to any delivery system for delivering materials or reagents for carrying out a method of the invention. Such delivery systems include systems that allow for the storage, transport, or delivery of reaction reagents (e.g., nucleotides, enzymes, nucleic acid templates, etc. in the appropriate containers) and/or supporting materials (e.g., buffers, written instructions for performing the reaction, etc.) from one location to another location. For example, kits include one or more enclosures (e.g., boxes) containing the relevant reaction reagents and/or supporting materials. Such contents may be delivered to the intended recipient together or separately. For example, a first container may contain an enzyme, while a second container contains nucleotides. In embodiments, the kit includes vessels containing one or more enzymes, primers, adaptors, or other reagents as described herein. Vessels may include any structure capable of supporting or containing a liquid or solid material and may include, tubes, vials, jars, containers, tips, etc. In embodiments, a wall of a vessel may permit the transmission of light through the wall. In embodiments, the vessel may be optically clear. The kit may include the enzyme and/or nucleotides in a buffer. In embodiments, the buffer includes an acetate buffer, 3-(N-morpholino) propanesulfonic acid (MOPS) buffer, N-(2-Acetamido)-2-aminoethanesulfonic acid (ACES) buffer, phosphate-buffered saline (PBS) buffer, 4-(2-hydroxyethyl)-1-piperazineethanesulfonic acid (HEPES) buffer, N-(1,1-Dimethyl-2-hydroxyethyl)-3-amino-2-hydroxypropanesulfonic acid (AMPSO) buffer, borate buffer (e.g., borate buffered saline, sodium borate buffer, boric acid buffer), 2-Amino-2-methyl-1,3-propanediol (AMPD) buffer, N-cyclohexyl-2-hydroxyl-3-aminopropanesulfonic acid (CAPSO) buffer, 2-Amino-2-methyl-1-propanol (AMP) buffer, 4-(Cyclohexylamino)-1-butanesulfonic acid (CABS) buffer, glycine-NaOH buffer, N-Cyclohexyl-2-aminoethanesulfonic acid (CHES) buffer, tris(hydroxymethyl)aminomethane (Tris) buffer, or a N-cyclohexyl-3-aminopropanesulfonic acid (CAPS) buffer. In embodiments, the buffer is a borate buffer. In embodiments, the buffer is a CHES buffer.

The methods and kits of the present disclosure may be applied, mutatis mutandis, to the sequencing of RNA, or to determining the identity of a ribonucleotide.

By aqueous solution herein is meant a liquid comprising at least 20 vol % water. In embodiments, aqueous solution includes at least 50%, for example at least 75 vol %, at least 95 vol %, above 98 vol %, or 100 vol % of water as the continuous phase.

The term “nucleic acid sequencing device” and the like means an integrated system of one or more chambers, ports, and channels that are interconnected and in fluid communication and designed for carrying out an analytical reaction or process, either alone or in cooperation with an appliance or instrument that provides support functions, such as sample introduction, fluid and/or reagent driving means, temperature control, detection systems, data collection and/or integration systems, for the purpose of determining the nucleic acid sequence of a template polynucleotide. Nucleic acid sequencing devices may further include valves, pumps, and specialized functional coatings on interior walls. Nucleic acid sequencing devices may include a receiving unit, or platen, that orients the flow cell such that a maximal surface area of the flow cell is available to be exposed to an optical lens. Other nucleic acid sequencing devices include those provided by Singular Genomics Systems, Inc. (e.g., G4™sequencer), Illumina™, Inc. (e.g. HiSeq™, MiSeq™, NextSeq™, or NovaSeq™systems), Life Technologies™(e.g. ABI PRISM™, or SOLiD™systems), Pacific Biosciences (e.g. systems using SMRT™Technology such as the Sequel™or RS II™systems), or Qiagen (e.g. Genereader™system).

A “nucleotide type”, as used herein, refers to a particular nucleobase of a nucleotide triphosphate. For example, a nucleotide type may be a purine nucleotide (i.e., adenine and guanine) or pyrimidine nucleotides (i.e., cytosine and thymine). In embodiments, a first nucleotide type is an adenine nucleotide, or analog thereof. In embodiments, a second nucleotide type is a guanine nucleotide, or analog thereof. In embodiments, a third nucleotide type is a cytosine nucleotide, or analog thereof. In embodiments, a fourth nucleotide type is a thymine nucleotide, or analog thereof. A “doublet” of nucleotide types, as used herein, may include, for example, a plurality of dATP and dCTP nucleotides; or a plurality of dATP and dGTP nucleotides; or a plurality of dATP and dTTP nucleotides; or a plurality of dCTP and dGTP nucleotides; or a plurality of dCTP and dTTP nucleotides; or a plurality of dGTP and dTTP nucleotides. A “triplet” of nucleotide types, as used herein, may include, for example a plurality of dATP, dTTP, and dCTP nucleotides; or a plurality of dATP, dTTP, and dGTP nucleotides; or a plurality of dTTP, dCTP, and dGTP nucleotides; or a plurality of dGTP, dCTP, and dATP nucleotides. The above-mentioned doublets and triplets are merely illustrative. One having skill in the art understands that a doublet includes any possible combination of two nucleotide types, and that a triplet includes any possible combination of three nucleotide types.

The term “characteristic signature” as used herein refers to a distinguishing feature or features used to identify an agent or event. In embodiments, the characteristic signature is associated with the identity of the nucleotide in a collection of nucleotides, wherein each nucleotide is associated with a different characteristic signature. For example a specific fluorescent emission is characteristic of a first nucleotide type (e.g., Alexa Fluor™647 is indicative of dATP). In embodiments, the characteristic signature is a change in pH. For example, the pH change that occurs due to release of H⁺ions during the incorporation of a single nucleotide reaction as detected using a Field-effect transistors (FET) or other suitable detection apparatus. In embodiments, the characteristic signature is a change in local charge density around the template nucleic acid. Methods for detecting electrical charges are known, including methods and systems such as field-effect transistors, dielectric spectroscopy, impedance measurements, and pH measurements, among others. Field-effect transistors include, but are not limited to, ion-sensitive field-effect transistors (ISFET), charge-modulated field-effect transistors, insulated-gate field-effect transistors, metal oxide semiconductor field-effect transistors and field-effect transistors fabricated using semiconducting single wall carbon nanotubes. In embodiments, the characteristic signature is detecting the absence of a label. For example, when the method includes the detection of four different nucleotides using fewer than four different labels. In embodiments, the characteristic signature is a fluorescent emission.

As used herein an “ordered cycle” refers to a process of events occurring according to a predetermined arrangement (e.g., events in successive, temporal, order). An ordered cycle may include a set of instructions or sequence useful to direct the series of events. For example, an ordered cycle includes a first element (e.g., a first reaction), followed by a second element (e.g., a second reaction), and so on, wherein the elements can appear multiple times and at different positions in the sequence of events. In embodiments, the ordered cycle is included in a software program. As used herein, the terms “software” and “firmware” are interchangeable, and include any computer program stored in memory for execution by a computer, including RAM memory, ROM memory, EPROM memory, EEPROM memory, and non-volatile RAM (NVRAM) memory.

As used herein a “non-cyclic sequence” is an arrangement of elements, wherein the arrangement of elements does not repeat (i.e., does not have a period). The Thue-Morse sequence, t = (tₙ)n≥₀, is a type of a non-cyclic sequence. In mathematics and computer science, a string is used to refer to a sequence of characters (e.g., a word). A string may be square, in that it has two repeating subunits (e.g., mama, murmur). Similarly, a string may be cube when three repeating subunits are present (e.g., hahaha). A string may be overlap, wherein one element repeats separated by a second element. For example, the word alfalfa is an overlap string by repeating ‘1f’ separated by ‘a.’ A Thue-Morse sequence does not include any overlap. A non-cyclic sequence is in contrast to a “cyclic sequence” (i.e., a periodic sequence) which refers to an arrangement of elements wherein the same elements are repeated over and over. For example, the sequence 1, 2, 1, 2, 1, 2 is periodic (i.e., the element [1, 2] is repeated three times). A cyclic sequence includes overlap.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### II. Compositions & Kits

In an aspect, provided herein are kits including one or more components of any of the various methods or compositions disclosed herein. In some embodiments, the kit is used for determining the identity of a base in a target nucleic acid by sequencing-by-synthesis, the kit including (a) a first sequencing mixture of deoxyribonucleotide triphosphates (dNTPs) including: a first plurality of dNTPs including a first label; and a second plurality of dNTPs including a second label; and (b) a second sequencing mixture of deoxyribonucleotide triphosphates (dNTPs) including: a third plurality of dNTPs including the first label; and a fourth plurality of dNTPs including the second label; wherein each of the first, second, third, and fourth pluralities of dNTPs is selected from the group consisting of dATP, dTTP, dCTP, and dGTP, and are different from each other; and wherein the first label and the second label are different labels and are distinguishable. In embodiments, the kit further includes instructions for use thereof. In embodiments, kits described herein include a polymerase. In embodiments, the polymerase is a DNA polymerase.

In an aspect is provided a kit for determining the identity of a base in a target nucleic acid by sequencing-by-synthesis, the kit including (a) a first mixture of deoxyribonucleotide triphosphates (dNTPs) including: a first plurality of dNTPs including a first label; and a second plurality of dNTPs including a second label; and (b) a second mixture of deoxyribonucleotide triphosphates (dNTPs) including: a third plurality of dNTPs comprising the first label; and a fourth plurality of dNTPs including the second label; wherein each of the first, second, third, and fourth pluralities of dNTPs is selected from the group consisting of dATP, dTTP, dCTP, and dGTP, and are different from each other; and wherein the first label and the second label are different labels and are distinguishable (e.g., distinguishable from each other).

In an aspect is provided a kit for determining the identity of a base in a target nucleic acid by sequencing-by-synthesis, the kit including (a) a first mixture of deoxyribonucleotide triphosphates (dNTPs) including: a first plurality of dNTPs including a first label; a second plurality of dNTPs including a second label; and a third plurality of dNTPs including a third label; (b) a second mixture of deoxyribonucleotide triphosphates (dNTPs) including: the second plurality of dNTPs including the first label; the third plurality of dNTPs including the second label; and a fourth plurality of dNTPs including the third label; (c) a third mixture of dNTPs including: the fourth plurality of dNTPs including the first label; the first plurality of dNTPs including the second label; and the second plurality of dNTPs including the third label; and (d) a fourth mixture of dNTPs including: the third plurality of dNTPs including the first label; the fourth plurality of dNTPs including the second label; and the first plurality of dNTPs including the third label; wherein each of the first, second, third, and fourth pluralities of dNTPs is selected from the group consisting of dATP, dTTP, dCTP, and dGTP, and are different from each other; and wherein the first label, the second label, and the third label are different labels and are distinguishable (e.g., distinguishable from each other). The kit may also include a template nucleic acid (DNA and/or RNA), one or more primer polynucleotides, nucleoside triphosphates (including, e.g., deoxyribonucleotides, ribonucleotides, particles, labeled nucleotides, and/or modified nucleotides), buffers, salts, and/or labels (e.g., fluorophores). In embodiments, the kit includes an array with particles already loaded into the wells. In embodiments, the particles are in a container. In embodiments, the particles are in aqueous suspension or as a powder within the container. The container may be a storage device or other readily usable vessel capable of storing and protecting the particles. The kit may also include a flow cell. In embodiments, kit includes the solid support and a flow cell carrier (e.g., a flow cell carrier as described in US 2021/0190668, which is incorporated herein by reference for all purposes).

As described herein, a mixture of deoxyribonucleotide triphosphates (dNTPs) may include a sequencing mixture (i.e., a mixture of dNTPs used during a cycle that includes detecting a characteristic signature indicating that one to three nucleotides have been incorporated into a primer), or a mixture of dNTPs may include an extension mixture (e.g., a mixture of dNTPs lacking a detectable label, and used during a cycle that does not include detecting a characteristic signature).

In an aspect, provided herein are kits including one or more components of any of the various methods or compositions disclosed herein. In some embodiments, the kit is used for determining the identity of a base in a target nucleic acid by sequencing-by-synthesis, the kit including (a) a first sequencing mixture of deoxyribonucleotide triphosphates (dNTPs) including: a first plurality of incorporable dNTPs including a first label; and a second plurality of dNTPs including a second label; and (b) a second sequencing mixture of deoxyribonucleotide triphosphates (dNTPs) including: a third plurality of dNTPs including the first label; and a fourth plurality of dNTPs including the second label; and (c) a third sequencing mixture including non-incorporating (e.g., non-hydrolyzable) dNTPs including: a first plurality of non-incorporating dNTPs; a second plurality of non-incorporating dNTPs; a third plurality of non-incorporating dNTPs; and a fourth plurality of non-incorporating dNTPs; wherein each of the first, second, third, and fourth pluralities of dNTPs is selected from the group consisting of dATP, dTTP, dCTP, and dGTP, and are different from each other; wherein the first label and the second label are different labels and are distinguishable; and wherein each of the first, second, third, and fourth pluralities of non-incorporating dNTPs is selected from the group consisting of a non-incorporable analog of dATP, a non-incorporable analog of dTTP, a non-incorporable analog of dCTP, and a non-incorporable analog of dGTP, and are different from each other. In embodiments, the kit further includes instructions for use thereof. In embodiments, kits described herein include a polymerase. In embodiments, the polymerase is a DNA polymerase.

In an aspect is provided a kit for determining the identity of a base in a target nucleic acid by sequencing-by-synthesis, the kit including (a) a first mixture of deoxyribonucleotide triphosphates (dNTPs) including: a first plurality of dNTPs including a first label; and a second plurality of dNTPs including a second label; and (b) a second mixture of deoxyribonucleotide triphosphates (dNTPs) including: a third plurality of dNTPs including the first label; and a fourth plurality of dNTPs including the second label; and (c) a third mixture including non-incorporating dNTPs including: a first plurality of non-incorporating dNTPs; a second plurality of non-incorporating dNTPs; a third plurality of non-incorporating dNTPs; and a fourth plurality of non-incorporating dNTPs; wherein each of the first, second, third, and fourth pluralities of dNTPs is selected from the group consisting of dATP, dTTP, dCTP, and dGTP, and are different from each other; wherein the first label and the second label are different labels and are distinguishable; and wherein each of the first, second, third, and fourth pluralities of non-incorporating dNTPs is selected from the group consisting of a non-incorporable analog of dATP, a non-incorporable analog of dTTP, a non-incorporable analog of dCTP, and a non-incorporable analog of dGTP, and are different from each other.

Generally, the kit includes one or more containers providing a composition and one or more additional reagents (e.g., a buffer suitable for polynucleotide extension). The kit may also include a template nucleic acid (DNA and/or RNA), one or more primer polynucleotides, nucleoside triphosphates (including, e.g., deoxyribonucleotides, ribonucleotides, labeled nucleotides, and/or modified nucleotides), buffers, salts, and/or labels (e.g., fluorophores).

In some embodiments of a kit herein, the dNTPs include a reversible terminator. In some embodiment, the reversible terminator is a 3′-reversible terminator. In other embodiments, the reversible terminator is a virtual terminator. In yet other embodiments, each nucleotide includes a 3′-reversible terminator and a detectable label.

In some embodiments of a kit herein, the dNTPs are non-hydrolyzable dNTPs (alternatively referred to as non-incorporating nucleotides). In embodiments, the non-hydrolyzable dNTPs include α-phosphate modified nucleotides, α,β nucleotide analogs, β-phosphate modified nucleotides, β-γ nucleotide analogs, γ-phosphate modified nucleotides, caged nucleotides, or dinucleotide analogs. In some embodiments, the non-hydrolyzable dNTPs include α,β-methylene-2′-deoxynucleoside 5′-triphosphate nucleotides. In embodiments, the non-hydrolyzable dNTPs include α-phosphate modified nucleotides. In embodiments, the non-hydrolyzable dNTPs include β-phosphate modified nucleotides. In embodiments, the non-hydrolyzable dNTPs include β-γ modified nucleotides. In embodiments, the non-hydrolyzable dNTPs include caged nucleotides. In embodiments, the non-hydrolyzable dNTPs include dinucleotide analogs. In embodiments, kits described herein include labeled non-hydrolyzable nucleotides. In embodiments, the non-hydrolyzable dNTPs lack a reversible terminator (e.g., the non-hydrolyzable dNTPs include a free 3′—OH).

In embodiments, kits described herein include labeled nucleotides including four differently labeled nucleotides, where the label identifies the type of nucleotide. For example, each of an adenine nucleotide, or analog thereof; a thymine nucleotide; a cytosine nucleotide, or analog thereof; and a guanine nucleotide, or analog thereof may be labelled with a different fluorescent label, or a different combination of labels. In embodiments, the adenine nucleotide, or analog thereof; a thymine nucleotide; a cytosine nucleotide, or analog thereof; and a guanine nucleotide, or analog thereof may be labelled with a different fluorescent label (or different combination of labels) and one may be unlabeled.

In embodiments, the kit includes labeled nucleotides including (a) four or fewer differently labeled nucleotides, wherein the label identifies the type of nucleotide, and (b) unlabeled nucleotides lacking a reversible terminator. In embodiments, the kit includes labeled nucleotides comprising four or fewer differently labeled nucleotides, wherein the label identifies the type of nucleotide.

In embodiments, kits described herein include unlabeled nucleotides lacking a reversible terminator. In embodiments, kits described herein include unlabeled nucleotides including a reversible terminator. In embodiments, kits described herein include labeled nucleotides including a reversible terminator. In embodiments, kits described herein include labeled nucleotides without a reversible terminator.

In embodiments, kits described herein include a polymerase. In embodiments, the polymerase is a DNA polymerase. In embodiments, the DNA polymerase is a thermophilic nucleic acid polymerase. In embodiments, the DNA polymerase is a modified archaeal DNA polymerase.

In an aspect, provided herein are reaction mixtures for use in accordance with any of the methods disclosed herein, and including one or more elements thereof. In embodiments, a reaction mixture includes labeled nucleotides including four differently labeled nucleotides, where the label identifies the type of nucleotide, unlabeled nucleotides lacking a reversible terminator; unlabeled nucleotides including a reversible terminator; and a polymerase.

In embodiments, the polymerase in the kit is a bacterial DNA polymerase, eukaryotic DNA polymerase, archaeal DNA polymerase, viral DNA polymerase, or phage DNA polymerases. Bacterial DNA polymerases include E. coli DNA polymerases I, II and III, IV and V, the Klenow fragment of E. coli DNA polymerase, Clostridium stercorarium (Cst) DNA polymerase, Clostridium thermocellum (Cth) DNA polymerase and Sulfolobus solfataricus (Sso) DNA polymerase. Eukaryotic DNA polymerases include DNA polymerases α, β, γ, δ, €, η, ζ, λ, σ, µ, and k, as well as the Revl polymerase (terminal deoxycytidyl transferase) and terminal deoxynucleotidyl transferase (TdT). Viral DNA polymerases include T4 DNA polymerase, phi-29 DNA polymerase, GA-1, phi-29-like DNA polymerases, PZA DNA polymerase, phi-15 DNA polymerase, Cpl DNA polymerase, Cpl DNA polymerase, T7 DNA polymerase, and T4 polymerase. Other useful DNA polymerases include thermostable and/or thermophilic DNA polymerases such as Thermus aquaticus (Taq) DNA polymerase, Thermus filiformis (Tfi) DNA polymerase, Thermococcus zilligi (Tzi) DNA polymerase, Thermus thermophilus (Tth) DNA polymerase, Thermus flavusu (Tfl) DNA polymerase, Pyrococcus woesei (Pwo) DNA polymerase, Pyrococcus furiosus (Pfu) DNA polymerase and Turbo Pfu DNA polymerase, Thermococcus litoralis (Tli) DNA polymerase, Pyrococcus sp. GB-D polymerase, Thermotoga maritima (Tma) DNA polymerase, Bacillus stearothermophilus (Bst) DNA polymerase, Pyrococcus Kodakaraensis (KOD) DNA polymerase, Pfx DNA polymerase, Thermococcus sp. JDF-3 (JDF-3) DNA polymerase, Thermococcus gorgonarius (Tgo) DNA polymerase, Thermococcus acidophilium DNA polymerase; Sulfolobus acidocaldarius DNA polymerase; Thermococcus sp. go N-7 DNA polymerase; Pyrodictium occultum DNA polymerase; Methanococcus voltae DNA polymerase; Methanococcus thermoautotrophicum DNA polymerase; Methanococcus jannaschii DNA polymerase; Desulfurococcus strain TOK DNA polymerase (D. Tok Pol); Pyrococcus abyssi DNA polymerase; Pyrococcus horikoshii DNA polymerase; Pyrococcus islandicum DNA polymerase; Thermococcus fumicolans DNA polymerase; Aeropyrum pernix DNA polymerase; and the heterodimeric DNA polymerase DP1/DP2. In embodiments, the polymerase is 3PDX polymerase as disclosed in U.S. 8,703,461, the disclosure of which is incorporated herein by reference. In embodiments, the polymerase is a reverse transcriptase. Exemplary reverse transcriptases include, but are not limited to, HIV-1 reverse transcriptase from human immunodeficiency virus type 1 (PDB 1HMV), HIV-2 reverse transcriptase from human immunodeficiency virus type 2, M-MLV reverse transcriptase from the Moloney murine leukemia virus, AMV reverse transcriptase from the avian myeloblastosis virus, or Telomerase reverse transcriptase. In embodiments, the polymerase is a mutant P. abyssi polymerase (e.g., such as a mutant P. abyssi polymerase described in WO 2018/148723 or WO 2020/056044, each of which are incorporated herein by reference for all purposes). In embodiments, the kit includes a strand-displacing polymerase. In embodiments, the kit includes a strand-displacing polymerase, such as a phi29 polymerase, phi29 mutant polymerase or a thermostable phi29 mutant polymerase.

In embodiments, the kit includes a buffered solution. Typically, the buffered solutions contemplated herein are made from a weak acid and its conjugate base or a weak base and its conjugate acid. For example, sodium acetate and acetic acid are buffer agents that can be used to form an acetate buffer. Other examples of buffer agents that can be used to make buffered solutions include, but are not limited to, Tris, Bicine, Tricine, HEPES, TES, MOPS, MOPSO and PIPES. Additionally, other buffer agents that can be used in enzyme reactions, hybridization reactions, and detection reactions are known in the art. In embodiments, the buffered solution can include Tris. With respect to the embodiments described herein, the pH of the buffered solution can be modulated to permit any of the described reactions. In some embodiments, the buffered solution can have a pH greater than pH 7.0, greater than pH 7.5, greater than pH 8.0, greater than pH 8.5, greater than pH 9.0, greater than pH 9.5, greater than pH 10, greater than pH 10.5, greater than pH 11.0, or greater than pH 11.5. In other embodiments, the buffered solution can have a pH ranging, for example, from about pH 6 to about pH 9, from about pH 8 to about pH 10, or from about pH 7 to about pH 9. In embodiments, the buffered solution can comprise one or more divalent cations. Examples of divalent cations can include, but are not limited to, Mg²⁺, Mn²⁺, Zn²⁺, and Ca²⁺. In embodiments, the buffered solution can contain one or more divalent cations at a concentration sufficient to permit hybridization of a nucleic acid. In embodiments, the buffer includes PEG (polyethylene glycol), PVP (polyvinylpyrrolidone), trehalose, ficoll, or dextran. In embodiments, the buffer includes additives such as Tween-20 or NP-40.

In embodiments, the kit includes a sequencing reaction mixture (e.g., a sequencing reaction mixture as described herein). In some embodiments, provided herein are reaction mixtures including labeled nucleotides including four or fewer differently labeled nucleotides, where the label identifies the type of nucleotide, unlabeled nucleotides lacking a reversible terminator; unlabeled nucleotides including a reversible terminator; and a polymerase.

In some embodiments, provided herein are kits including a plurality of different sequencing solutions. In some embodiments, the plurality of different sequencing solutions include different combinations of fewer than four nucleotide types. The plurality of sequencing solutions having different combinations of fewer than four nucleotide types may have the same or different number of nucleotide types, and may be incompletely overlapping or non-overlapping in nucleotide types. For example, a first sequencing solution may include two types of nucleotides, and the second solution may include the same two nucleotide types and a third nucleotide type. As a further example, a first sequencing solution may include two types of nucleotides (e.g., T and C), and a second sequencing solution may include two different types of nucleotides (e.g., A and G). In some embodiments, in addition to the plurality of sequencing solutions having different combinations of fewer than four nucleotide types, the kits may include one or more sequencing solutions with a single nucleotide type, and/or a sequencing solution with four nucleotide types (e.g., A, C, G, and T). In some embodiments, the kits may include a sequencing solution with four nucleotide types, wherein one or more of the nucleotide types are non-incorporating (i.e., non-hydrolyzable). In embodiments, the kits may include a sequencing solution with four nucleotide types, wherein two of the nucleotide types are non-incorporating. For example, a sequencing solution may include two types of nucleotides including a reversible terminator (e.g., T and C), and two types of non-incorporating nucleotides (e.g., A and G). Further examples of different sequencing solutions are provided herein, including in connections with various methods of the present disclosure.

### III. Methods

In an aspect is provided a method for extending a primer hybridized to a nucleic acid template, the method including: (a) contacting the primer with a first extension solution in the presence of a polymerase; (b) contacting the primer with a second extension solution in the presence of a polymerase thereby extending the primer by a single nucleotide; wherein: the first extension solution includes a first doublet of nucleotide types and the second extension solution includes a second doublet of nucleotide types, wherein the first doublet of nucleotide types have no nucleotide types in common with the second doublet of nucleotide types; and (c) repeating steps (a) and (b), wherein each repetition of steps (a) and (b) is a cycle, wherein each cycle is performed two or more (e.g., at least 2, 5, 10, 15, 20, 25, or 30) times thereby performing a series of cycles, wherein each cycle is a first ordered cycle or a second ordered cycle, wherein the first ordered cycle contacts the primer with the first extension solution first and the second extension solution second, wherein the second ordered cycle contacts the primer with the second extension solution first and the first extension solution second, wherein the series of cycles is performed according to a non-cyclic sequence.

In an aspect is provided a method for extending a primer hybridized to a nucleic acid template, the method including: (a) contacting the primer with a first extension solution in the presence of a polymerase; (b) contacting the primer with a second extension solution in the presence of a polymerase thereby extending the primer by a single nucleotide; wherein: the first extension solution includes a first triplet of nucleotide types and the second extension solution includes a second triplet of nucleotide types, wherein the first triplet of nucleotide types has one or two nucleotide types in common with the second triplet of nucleotide types; and (c) repeating steps (a) and (b), wherein each repetition of steps (a) and (b) is a cycle, wherein each cycle is performed two or more (e.g., at least 2, 5, 10, 15, 20, 25, or 30) times thereby performing a series of cycles, wherein each cycle is a first ordered cycle or a second ordered cycle, wherein the first ordered cycle contacts the primer with the first extension solution first and the second extension solution second, wherein the second ordered cycle contacts the primer with the second extension solution first and the first extension solution second, wherein the series of cycles is performed according to a non-cyclic sequence. In embodiments, said first triplet of nucleotide types has one or two nucleotide types different from said second triple of nucleotide types.

In an aspect is provided a method for extending a primer hybridized to a nucleic acid template, the method including: (a) contacting the primer with a first extension solution in the presence of a polymerase; (b) contacting the primer with a second extension solution in the presence of a polymerase thereby extending the primer by a single nucleotide; wherein: the first extension solution includes a first doublet of nucleotide types and the second extension solution includes a second triplet of nucleotide types, wherein the first doublet of nucleotide types has one or two nucleotide types in common with the second triplet of nucleotide types; and (c) repeating steps (a) and (b), wherein each repetition of steps (a) and (b) is a cycle, wherein each cycle is performed two or more (e.g., at least 2, 5, 10, 15, 20, 25, or 30) times performing a series of cycles, wherein each cycle is a first ordered cycle or a second ordered cycle, wherein the first ordered cycle contacts the primer with the first extension solution first and the second extension solution second, wherein the second ordered cycle contacts the primer with the second extension solution first and the first extension solution second, wherein the series of cycles is performed according to a non-cyclic sequence.

In an aspect is provided a method for extending a primer hybridized to a nucleic acid template, the method including: (a) contacting the primer with a first extension solution in the presence of a polymerase; (b) contacting the primer with a second extension solution in the presence of a polymerase thereby extending the primer by a single nucleotide; wherein: the first extension solution includes a first triplet of nucleotide types and the second extension solution includes a second doublet of nucleotide types, wherein the first triplet of nucleotide types has one or two nucleotide types in common with the second doublet of nucleotide types; and (c) repeating steps (a) and (b), wherein each repetition of steps (a) and (b) is a cycle, wherein each cycle is performed two or more (e.g., at least 2, 5, 10, 15, 20, 25, or 30) times thereby performing a series of cycles, wherein each cycle is a first ordered cycle or a second ordered cycle, wherein the first ordered cycle contacts the primer with the first extension solution first and the second extension solution second, wherein the second ordered cycle contacts the primer with the second extension solution first and the first extension solution second, wherein the series of cycles is performed according to a non-cyclic sequence.

In embodiments, each cycle is performed 2, 3, 4, 5, 6, 7, 8, 9, 10, 11, 12, 13, 14, 15, 16, 17, 18, 19, 20 or more times thereby performing a series of cycles. In embodiments, each cycle is performed 20, 30, 40, 50, 60, 70, 80, 90, 100, 110, 120, 130, 140, 150, 160, 170, 180, 190, 200 or more times thereby performing a series of cycles. In embodiments, each cycle is performed at least 20 times. In embodiments, each cycle is performed at least 30 times. In embodiments, each cycle is performed at least 40 times. In embodiments, each cycle is performed at least 50 times. In embodiments, each cycle is performed at least 60 times. In embodiments, each cycle is performed at least 70 times. In embodiments, each cycle is performed at least 80 times. In embodiments, each cycle is performed at least 90 times. In embodiments, each cycle is performed at least 100 times. In embodiments, each cycle is performed at least 110 times. In embodiments, each cycle is performed at least 120 times. In embodiments, each cycle is performed at least 130 times. In embodiments, each cycle is performed at least 140 times. In embodiments, each cycle is performed at least 150 times. In embodiments, each cycle is performed at least 160 times. In embodiments, each cycle is performed at least 170 times. In embodiments, each cycle is performed at least 180 times. In embodiments, each cycle is performed at least 190 times. In embodiments, each cycle is performed at least 200 times.

As described herein, a cycle may refer to a sequencing cycle (i.e., a cycle that includes detecting a characteristic signature indicating that one to three nucleotides have been incorporated into the primer), or a cycle may refer to an extension cycle (e.g., a dark cycle, wherein the cycle does not include detecting a characteristic signature).

In embodiments, the first doublet of nucleotide types has one nucleotide type in common with the second triplet of nucleotide types. In embodiments, the first doublet of nucleotide types has two nucleotide types in common with the second triplet of nucleotide types. In embodiments, the first triplet of nucleotide types has one nucleotide type in common with the second doublet of nucleotide types. In embodiments, the first triplet of nucleotide types has two nucleotide types in common with the second doublet of nucleotide types. In embodiments, the first triplet of nucleotide types has one nucleotide type in common with the second triplet of nucleotide types. In embodiments, the first triplet of nucleotide types has two nucleotide types in common with the second triplet of nucleotide types.

In embodiments, prior to step c), the method further includes detecting a characteristic signature indicating that the one to three nucleotides have been incorporated into the primer. In embodiments, prior to step b), the method further includes detecting a characteristic signature indicating that the one to three nucleotides have been incorporated into the primer.

In an aspect is a method for sequencing a nucleic acid template, the method including: a) hybridizing one or more sequencing primers to a nucleic acid template; b) executing a plurality of sequencing cycles, each cycle including (i) contacting the nucleic acid template with a sequencing solution in the presence of a polymerase, wherein the sequencing solutions of at least two sequencing cycles include a different combination of fewer than four nucleotide types, wherein each nucleotide of each nucleotide type includes a reversible terminator; and (ii) detecting a characteristic signature indicating that the nucleotide has been incorporated into the sequencing primer.

In embodiments, sequencing includes sequencing-by-synthesis, sequencing-by-binding, sequencing by ligation, or pyrosequencing. In embodiments, generating a first sequencing read or a second sequencing read includes a sequencing by synthesis process. In embodiments, generating a first sequencing read or a second sequencing read includes a sequencing-by-binding. As used herein, “sequencing-by-binding” refers to a sequencing technique wherein specific binding of a polymerase and cognate nucleotide to a primed template nucleic acid molecule (e.g., blocked primed template nucleic acid molecule) is used for identifying the next correct nucleotide to be incorporated into the primer strand of the primed template nucleic acid molecule. The specific binding interaction need not result in chemical incorporation of the nucleotide into the primer. In some embodiments, the specific binding interaction can precede chemical incorporation of the nucleotide into the primer strand or can precede chemical incorporation of an analogous, next correct nucleotide into the primer. Thus, detection of the next correct nucleotide can take place without incorporation of the next correct nucleotide. As used herein, the “next correct nucleotide” (sometimes referred to as the “cognate” nucleotide) is the nucleotide having a base complementary to the base of the next template nucleotide. The next correct nucleotide will hybridize at the 3′-end of a primer to complement the next template nucleotide. The next correct nucleotide can be, but need not necessarily be, capable of being incorporated at the 3′ end of the primer. For example, the next correct nucleotide can be a member of a ternary complex that will complete an incorporation reaction or, alternatively, the next correct nucleotide can be a member of a stabilized ternary complex that does not catalyze an incorporation reaction. A nucleotide having a base that is not complementary to the next template base is referred to as an “incorrect” (or “non-cognate”) nucleotide. In embodiments, sequencing includes generating a sequencing read. A variety of sequencing methodologies can be used such as sequencing-by-synthesis (SBS), pyrosequencing, sequencing by ligation (SBL), or sequencing by hybridization (SBH). Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 11(1), 3-11 (2001); Ronaghi et al. Science 281(5375), 363 (1998); U.S. Pat. Nos. 6,210,891; 6,258,568; and. 6,274,320, each of which is incorporated herein by reference in its entirety). In pyrosequencing, released Ppi can be detected by being converted to adenosine triphosphate (ATP) by ATP sulfurylase, and the level of ATP generated can be detected via light produced by luciferase. In this manner, the sequencing reaction can be monitored via a luminescence detection system. In both SBL and SBH methods, target nucleic acids, and amplicons thereof, that are present at features of an array are subjected to repeated cycles of oligonucleotide delivery and detection. SBL methods, include those described in Shendure et al. Science 309:1728-1732 (2005); U.S. Pat. Nos. 5,599,675; and 5,750,341, each of which is incorporated herein by reference in its entirety; and the SBH methodologies are as described in Bains et al., Journal of Theoretical Biology 135(3), 303-7 (1988); Drmanac et al., Nature Biotechnology 16, 54-58 (1998); Fodor et al., Science 251(4995), 767-773 (1995); and WO 1989/10977, each of which is incorporated herein by reference in its entirety.

In SBS, extension of a nucleic acid primer along a nucleic acid template is monitored to determine the sequence of nucleotides in the template. The underlying chemical process can be catalyzed by a polymerase, wherein fluorescently labeled nucleotides are added to a primer (thereby extending the primer) in a template dependent fashion such that detection of the order and type of nucleotides added to the primer can be used to determine the sequence of the template. A plurality of different nucleic acid fragments that have been attached at different locations of an array can be subjected to an SBS technique under conditions where events occurring for different templates can be distinguished due to their location in the array. In embodiments, the sequencing step includes annealing and extending a sequencing primer to incorporate a detectable label that indicates the identity of a nucleotide in the target polynucleotide, detecting the detectable label, and repeating the extending and detecting steps. In embodiments, the methods include sequencing one or more bases of a target nucleic acid by extending a sequencing primer hybridized to a target nucleic acid (e.g., an amplification product produced by the amplification methods described herein). In embodiments, the sequencing step may be accomplished by a sequencing-by-synthesis (SBS) process. In embodiments, sequencing comprises a sequencing by synthesis process, where individual nucleotides are identified iteratively, as they are polymerized to form a growing complementary strand. In embodiments, nucleotides added to a growing complementary strand include both a label and a reversible chain terminator that prevents further extension, such that the nucleotide may be identified by the label before removing the terminator to add and identify a further nucleotide. Such reversible chain terminators include removable 3′ blocking groups, for example as described in U.S. Pat. Nos. 10,738,072, 7,541,444 and 7,057,026. Once such a modified nucleotide has been incorporated into the growing polynucleotide chain complementary to the region of the template being sequenced, there is no free 3′—OH group available to direct further sequence extension and therefore the polymerase cannot add further nucleotides. Once the identity of the base incorporated into the growing chain has been determined, the 3′ block may be removed to allow addition of the next successive nucleotide. By ordering the products derived using these modified nucleotides it is possible to deduce the DNA sequence of the DNA template. For example, a nucleotide analog having a reversible terminator moiety can be added to a primer such that subsequent extension cannot occur until a deblocking agent (e.g., a reducing agent) is delivered to remove the moiety. Thus, for embodiments that use reversible termination, a deblocking reagent (e.g., a reducing agent) can be delivered to the flow cell (before, during, or after detection occurs). Washes can be carried out between the various delivery steps as needed. The cycle can then be repeated N times to extend the primer by N nucleotides, thereby detecting a sequence of length N. Example SBS procedures, fluidic systems and detection platforms that can be readily adapted for use with an array produced by the methods of the present disclosure are described, for example, in Bentley et al., Nature 456:53-59 (2008), U.S Pat. Publication 2018/0274024, WO 2017/205336, U.S Pat. Publication 2018/0258472, each of which are incorporated herein in their entirety for all purposes.

Sequencing includes, for example, detecting a sequence of signals. Examples of sequencing include, but are not limited to, sequencing by synthesis (SBS) processes in which reversibly terminated nucleotides carrying fluorescent dyes are incorporated into a growing strand, complementary to the target strand being sequenced. In embodiments, the nucleotides are labeled with up to four unique fluorescent dyes. In embodiments, the nucleotides are labeled with at least two unique fluorescent dyes. In embodiments, the readout is accomplished by epifluorescence imaging. A variety of sequencing chemistries are available, non-limiting examples of which are described herein.

For example, a nucleotide type is determined by the nucleobase. For example, a nucleotide type may be a purine nucleotide (i.e., adenine and guanine) or pyrimidine nucleotides (i.e., cytosine and thymine). In embodiments, a first nucleotide type is an adenine nucleotide, or analog thereof. In embodiments, a second nucleotide type is a guanine nucleotide, or analog thereof. In embodiments, a third nucleotide type is a cytosine nucleotide, or analog thereof. In embodiments, a fourth nucleotide type is a thymine nucleotide, or analog thereof.

In embodiments, the second extension solution includes a different combination of nucleotide types than the first extension solution. In embodiments, the second extension solution includes the same combination of nucleotide types as the first extension solution.

In embodiments, the first extension solution includes a first doublet of nucleotide types and the second extension solution includes a second doublet of nucleotide types, wherein the first doublet of nucleotide types have no nucleotide types in common with the second doublet of nucleotide types. In embodiments, the first extension solution includes a first triplet of nucleotide types and the second extension solution includes a second triplet of nucleotide types, wherein the first triplet of nucleotide types has one or two nucleotide types in common with the second triplet of nucleotide types. In embodiments, the first extension solution includes a first doublet of nucleotide types and the second extension solution includes a second triplet of nucleotide types, wherein the first doublet of nucleotide types has one or two nucleotide types in common with the second triplet of nucleotide types. In embodiments, the first extension solution includes a first triplet of nucleotide types and the second extension solution includes a second doublet of nucleotide types, wherein the first triplet of nucleotide types has one or two nucleotide types in common with the second doublet of nucleotide types. In embodiments, the first extension solution includes a first triplet of nucleotide types and the second extension solution includes a second triplet of nucleotide types, wherein the first triplet of nucleotide types has one or two nucleotide types in common with the second triplet of nucleotide types. In embodiments, the first extension solution includes a first doublet of nucleotide types and the second extension solution includes a second triplet of nucleotide types, wherein the first doublet of nucleotide types has two nucleotide types in common with the second triplet of nucleotide types. In embodiments, the first extension solution includes a first triplet of nucleotide types and the second extension solution includes a second doublet of nucleotide types, wherein the first triplet of nucleotide types has two nucleotide types in common with the second doublet of nucleotide types.

In embodiments, each cycle (e.g., each extension or sequencing cycle) is performed at least 20 times, 30 times, at least 40 times, or at least 50 times. In embodiments, the series of cycles includes at least 2 cycles. In embodiments, the series of cycles includes at least 5 cycles. In embodiments, the series of cycles includes at least 8 cycles. In embodiments, the series of cycles includes at least 10 cycles. In embodiments, the series of cycles includes at least 15 cycles. In embodiments, the series of cycles includes at least 20 cycles. In embodiments, the series of cycles includes at least 25 cycles. In embodiments, the series of cycles includes at least 30 cycles. In embodiments, the series of cycles includes at least 40 cycles, or at least 50 cycles. In embodiments, the series of cycles includes at least 75 cycles, at least 100 cycles, at least 150 cycles, at least 200 cycles, at least 250 cycles, at least 300 cycles, at least 350 cycles, at least 400 cycles, at least 450 cycles, or at least 500 cycles. In embodiments, the series of cycles includes greater than 2 cycles. In embodiments, the series of cycles includes greater than 5 cycles. In embodiments, the series of cycles includes greater than 8 cycles. In embodiments, the series of cycles includes greater than 10 cycles. In embodiments, the series of cycles includes greater than 15 cycles. In embodiments, the series of cycles includes greater than 20 cycles. In embodiments, the series of cycles includes greater than 25 cycles. In embodiments, the series of cycles includes greater than 30 cycles. In embodiments, the series of cycles includes greater than 40 cycles, or greater than 50 cycles. In embodiments, the series of cycles includes greater than 75 cycles, greater than 100 cycles, greater than 150 cycles, greater than 200 cycles, greater than 250 cycles, greater than 300 cycles, greater than 350 cycles, greater than 400 cycles, greater than 450 cycles, or greater than 500 cycles.

In embodiments, the series of cycles includes about 2 to about 5 cycles. In embodiments, the series of cycles includes about 5 to about 10 cycles. In embodiments, the series of cycles includes about 10 to about 20 cycles. In embodiments, the series of cycles includes about 20 to about 50 cycles. In embodiments, the series of cycles includes about 50 to about 100 cycles. In embodiments, the series of cycles includes about 10 to about 100 cycles. In embodiments, the series of cycles includes about 100 cycles to about 200 cycles. In embodiments, the series of cycles includes about 100 cycles to about 300 cycles. In embodiments, the series of cycles includes about 250 to about 400 cycles. In embodiments the series of cycles includes about 250 to about 500 cycles. In embodiments, the series of cycles includes about 100 to about 500 cycles.

In embodiments, the nucleotide types of the first extension solution and the nucleotide types of the second extension solution differ across one or more cycles. In embodiments, the nucleotide types of the first extension solution and the nucleotide types of the second extension solution are the same across one or more cycles.

In embodiments, prior to detecting the characteristic signature, the method further includes contacting the primer with a dark solution, wherein the dark solution includes a plurality of unlabeled, 3′-reversibly terminated dATPs, dCTPs, dTTPs, or dGTPs. In embodiments, the nucleotide types of the dark solution are the same as the nucleotide types used in the sequencing solution.

In embodiments, the non-cyclic sequence includes a non-cyclic binary or non-cyclic ternary sequence. In embodiments, the non-cyclic sequence includes a non-cyclic binary sequence. In embodiments, the non-cyclic sequence includes a non-cyclic ternary sequence. In embodiments, the non-cyclic sequence includes a Thue-Morse sequence. In embodiments, the non-cyclic sequence is a pseudorandom sequence.

In embodiments, at least one nucleotide type of the first extension solution, the second extension solution, or both the first extension solution and the second extension solution is a non-incorporating nucleotide type. In embodiments, at least one nucleotide type of the first extension solution, the second extension solution, or both the first extension solution and the second extension solution is a non-incorporating nucleotide type and the remaining one or more nucleotide types include a reversible terminator. In embodiments, two nucleotide types of the first extension solution, the second extension solution, or both the first extension solution and the second extension solution are non-incorporating nucleotide types. In embodiments, greater than 10%, 20%, 30%, 40%, or 50% of the cycles include a first extension solution, a second extension solution, or both a first extension solution and a second extension solution that includes at least one non-incorporating nucleotide type. In embodiments, the first extension solution further comprises a non-incorporating nucleotide type (i.e., the extension solution includes a total of three nucleotide types, two of which are capable of being incorporated and/or detected).

In an aspect is a method for sequencing a nucleic acid template, the method including: a) hybridizing one or more sequencing primers to a nucleic acid template; b) executing a plurality of sequencing cycles, each cycle including (i) contacting the nucleic acid template with a sequencing solution in the presence of a polymerase, wherein the sequencing solutions of at least two sequencing cycles include a different combination of a plurality of four different nucleotide types, wherein at least one nucleotide type is a non-incorporating nucleotide type, and the remaining nucleotide types each include a reversible terminator; and (ii) detecting a characteristic signature indicating that the nucleotide has been incorporated into the sequencing primer. In embodiments, the two sequencing cycles are two sequential sequencing cycles. In other embodiments, the two sequencing cycles are two non-sequential sequencing cycles.

In an aspect is provided a method of enzymatic synthesis of a polynucleotide, the method including: a) hybridizing a primer to a first primer region of a template strand comprising universal base analogs (e.g., referred to herein as a universal template strand), wherein the primer is at least partially complementary to the first primer region; b) contacting the universal template strand with a modified nucleotide including a 3′-reversible terminator according to a flow order, wherein the flow order is selected according to a predetermined polynucleotide sequence; c) and in the presence of a polymerase, incorporating the nucleotide into the primer hybridized to the template strand; and d) repeating steps b)-c) to synthesize the polynucleotide. In embodiments, contacting the template strand with a modified nucleotide including a 3′-reversible terminator is performed according to a predetermined flow order. In embodiments, contacting the universal template strand with a modified nucleotide including a 3′-reversible terminator is performed according to a pseudorandom sequence flow order.

In an aspect is provided a method of synthesizing a plurality of polynucleotides having different, predetermined sequences, the method including: a) hybridizing primers to primer regions of a plurality of template strands comprising universal base analogs (e.g., referred to herein as universal template strands), wherein the primers are at least partially complementary to the primer regions, wherein the universal template strands are bound to a solid substrate; b) contacting the plurality of universal template strands with a modified nucleotide including a 3′-reversible terminator according to a flow order, wherein the flow order is selected according to a predetermined polynucleotide sequence; c) and in the presence of a polymerase, incorporating the nucleotide into the primers hybridized to the subset of the template strands; and d) repeating steps b)-c) with variations in the subset of the universal template strands and in a base of the modified nucleotide including a 3′ reversible terminator to synthesize the plurality of polynucleotides having different, predetermined sequences. In embodiments, contacting the plurality of template strands with a modified nucleotide including a 3′ reversible terminator is performed according to a predetermined flow order. In embodiments, contacting the plurality of universal template strands with a modified nucleotide including a 3′ reversible terminator is performed according to a pseudorandom sequence flow order.

In embodiments, the template strand includes at least a subset of bases that are not universal base analogs. In embodiments, at least 1% of bases in the template strand are not universal base analogs. In embodiments, at least 2% of bases in the template strand are not universal base analogs. In embodiments, at least 3% of bases in the template strand are not universal base analogs. In embodiments, at least 4% of bases in the template strand are not universal base analogs. In embodiments, at least 5% of bases in the template strand are not universal base analogs.

In embodiments, the template strand includes at least 95% universal base analogs. In embodiments, the template strand includes at least 99% universal base analogs. In embodiments, the template strand includes at least 95%, at least 96%, at least 97%, at least 98%, or at least 99% universal base analogs. In embodiments, the template strand includes greater than 95% universal base analogs. In embodiments the template strand includes greater than 99% universal base analogs. In embodiments, the template strand includes greater than 95%, greater than 96%, greater than 97%, greater than 98%, or greater than 99% universal base analogs.

In embodiments, the template strand includes a mixture of native nucleotides and universal base analogs. In embodiments, the template strand includes 5% native nucleotides and 95% universal base analogs. In embodiments, the template strand includes 4% native nucleotides and 96% universal base analogs. In embodiments, the template strand includes 3% native nucleotides and 97% universal base analogs. In embodiments, the template strand includes 2% native nucleotides and 98% universal base analogs. In embodiments, the template strand includes 1% native nucleotides and 99% universal base analogs. In embodiments, the template strand includes more than 5% native nucleotides and less than 95% universal base analogs. In embodiments, the template strand includes more than 4% native nucleotides and less than 96% universal base analogs. In embodiments, the template strand includes more than 3% native nucleotides and less than 97% universal base analogs. In embodiments, the template strand includes more than 2% native nucleotides and less than 98% universal base analogs. In embodiments, the template strand includes more than 1% native nucleotides and less than 99% universal base analogs. In embodiments, the template strand includes less than 1% native nucleotides and greater than 99% universal base analogs. In embodiments, the template strand includes less than 2% native nucleotides and greater than 98% universal base analogs. In embodiments, the template strand includes less than 3% native nucleotides and greater than 97% universal base analogs. In embodiments, the template strand includes less than 4% native nucleotides and greater than 96% universal base analogs. In embodiments, the template strand includes less than 5% native nucleotides and greater than 95% universal base analogs.

In some embodiments of a method herein, the predetermined flow order includes a non-cyclic binary or non-cyclic ternary sequence. In some embodiments of a method herein, the predetermined flow order includes a Thue-Morse sequence. In some embodiments, the predetermined flow order includes a de Bruijn sequence. In some embodiments, the predetermined flow order includes a Samba sequence. In some embodiments, the predetermined flow order includes a Gafieira sequence.

In embodiments, the template strand includes a region consisting of a mixture of natural bases and the universal base analogs. In embodiments, the template strand includes a homopolymeric sequence (e.g., a repetitive nucleic acid sequence and/or a tandemly repeating sequence unit). Approximately 1.43 million homopolymeric sequences exist in the human exome, with the size of 4-mer and up, and are believed to influence transcriptional regulation and recombination. A dinucleotide repeat is when two nucleotides are repeated, e.g., ACACAC) and instability including additions or truncations of repeating units is typically found in colon cancer. When three nucleotides are repeated, it is referred to as a trinucleotide repeat (e.g., CAGCAG), and abnormalities are correlated with Fragile X syndrome and Huntington’s disease.

In embodiments, the primer includes a 3′ blocking moiety. In embodiments the blocking moiety is thermolabile, acid-labile, redox-labile, or photolabile. In further embodiments, the 3′ blocking moiety is removed from the primer prior to contacting the template strand with a modified nucleotide including a 3′ reversible terminator. In embodiments, the polymerase is a DNA-dependent DNA polymerase, for example, terminal deoxynucleotidyl transferase (TdT). In embodiments, the backbone of the universal template strand includes peptide nucleic acids, bridged nucleic acids, locked nucleic acids, or ribose phosphate with a 2′-deoxy substitution. In embodiments, the universal template strand further includes a second primer region, and the method further includes contacting the universal template strand with a mixture of nucleotides without blocking moieties.

In embodiments, universal template strands are attached to a solid substrate. The universal template strands may be attached by any conventional technique for attaching polynucleotides sequences to solid substrates. For example, the surface of the solid substrate may be coated with linker molecules that in turn attach to an end of the universal template strands. As a further example, the surface of the solid substrate array may be functionalized through silanization or by coating with agarose. This creates a solid substrate that is coated with a plurality of anchor sequences. In some implementations, the solid substrate may be a microelectrode array. The solid substrate that is coated with universal template strands may be reused multiple times.

In some embodiments, the method includes executing a plurality of sequencing cycles by consecutively contacting the nucleic acid template with a first sequencing solution, followed by consecutively contacting the nucleic acid template with a second sequencing solution, wherein the first sequencing solution is different than the second sequencing solution. In the context of the methods described herein, a “plurality” of sequencing cycles may refer to either consecutively contacting the nucleic acid template with identical sequencing solutions, or consecutively contacting the nucleic acid template with different sequencing solutions. Furthermore, in the context of the methods described herein, a “plurality” of sequencing cycles may refer to either consecutively contacting the nucleic acid template with identical sequencing conditions, or consecutively contacting the nucleic acid template with different sequencing conditions. In embodiments, the method includes repeating contact with the first sequencing solution prior to introduction of the second sequencing solution.

In embodiments of a method herein, consecutively contacting includes contacting the nucleic acid template 2 to 10 times with a first or second sequencing solution. In other embodiments of a method herein, consecutively contacting includes contacting the nucleic acid template 2 to 4 times with a first or second sequencing solution. In some embodiments of a method herein, consecutively contacting includes contacting the nucleic acid template 2 times with a first or second sequencing solution. In embodiments, consecutively contacting includes contacting the nucleic acid template 3 times with a first or second sequencing solution. In embodiments, consecutively contacting includes contacting the nucleic acid template 4 times with a first or second sequencing solution. In embodiments, consecutively contacting includes contacting the nucleic acid template 5 times with a first or second sequencing solution. In embodiments, consecutively contacting includes contacting the nucleic acid template 6 times with a first or second sequencing solution. In embodiments, consecutively contacting includes contacting the nucleic acid template 7 times with a first or second sequencing solution. In embodiments, consecutively contacting includes contacting the nucleic acid template 8 times with a first or second sequencing solution. In embodiments, consecutively contacting includes contacting the nucleic acid template 9 times with a first or second sequencing solution. In embodiments, consecutively contacting includes contacting the nucleic acid template 10 times with a first or second sequencing solution. In embodiments, consecutively contacting includes contacting the nucleic acid template more than 10 times with a first or second sequencing solution.

In embodiments of a method herein, the sequencing solutions include a first nucleotide type (e.g., dA) including a reversible terminator and a second nucleotide type (e.g., dT) including a reversible terminator, and two non-incorporating nucleotide types (e.g., dC and dG). In embodiments, the sequencing solutions include a first nucleotide type (e.g., dA) including a reversible terminator, a second nucleotide type (e.g., dT) including a reversible terminator, and a third nucleotide type (e.g., dC) including a reversible terminator, and one non-incorporating nucleotide type (e.g., dG). In embodiments, the sequencing solutions include a first nucleotide type (e.g., dA) including a reversible terminator and three non-incorporating nucleotide types (e.g., dT, dC, and dG).

In embodiments of a method herein, each of a plurality of the sequencing solutions includes a different combination of two nucleotide types. In embodiments of a method herein, each of a plurality of the sequencing solutions includes a different combination of four nucleotide types. In embodiments of a method herein, each of a plurality of the sequencing solutions includes a different combination of two non-incorporating nucleotide types. In embodiments of a method herein, each of a plurality of the sequencing solutions includes a different combination of two non-incorporating nucleotide types and two nucleotide types including a reversible terminator. In embodiments of a method herein, each of a plurality of the sequencing solutions includes a different combination of one non-incorporating nucleotide type and three nucleotide types including a reversible terminator. In embodiments of a method herein, each of a plurality of the sequencing solutions includes a different combination of three non-incorporating nucleotide types and one nucleotide type including a reversible terminator. In some embodiments of a method herein, each of a plurality of the sequencing solutions includes a different combination of three nucleotide types. In embodiments, the plurality of sequencing solutions include one or more sequencing solutions with different combinations of two nucleotide types, and one or more sequencing solutions with different combinations of three nucleotide types. In embodiments, the plurality of sequencing solutions include one or more sequencing solutions with different combinations of two nucleotide types, and one or more sequencing solutions with different combinations of four nucleotide types. In embodiments, the plurality of sequencing solutions include one or more sequencing solutions with different combinations of two nucleotide types including a reversible terminator and two non-incorporating nucleotide types, wherein the two nucleotide types including a reversible terminator are different than the two non-incorporating nucleotide types. In embodiments of a method herein, each of a plurality of the sequencing solutions includes a different combination of three nucleotide types including a reversible terminator and one non-incorporating nucleotide type, wherein each of the nucleotide types including a reversible terminator are different than the non-incorporating nucleotide type. In embodiments of a method herein, each of a plurality of the sequencing solutions includes a different combination of one nucleotide type including a reversible terminator and three non-incorporating nucleotide types, wherein the nucleotide type including the reversible terminator is different than the non-incorporating nucleotide types.

In embodiments of a method herein, the sequencing solutions of at least two sequencing cycles include two nucleotide types, each including a reversible terminator, and two non-incorporating nucleotide types. In embodiments, the sequencing solutions of at least two sequencing cycles include three nucleotide types, each including a reversible terminator, and one non-incorporating nucleotide type. In embodiments, the sequencing solutions of at least two sequencing cycles include one nucleotide type including a reversible terminator and three non-incorporating nucleotide types.

In embodiments of a method herein, each of a plurality of the sequencing solutions includes a randomly determined combination of less than four nucleotide types. In embodiments of a method herein, each of a plurality of the sequencing solutions includes a randomly determined combination of three nucleotide types. In other embodiments of a method herein, each of a plurality of the sequencing solutions includes a randomly determined combination of two nucleotide types. In embodiments, the plurality of sequencing solutions include one or more sequencing solutions with a randomly determined combination of two nucleotide types, and one or more sequencing solutions with a randomly determined combination of three nucleotide types.

In embodiments of a method herein, greater than 10%, 20%, 30%, 40%, or 50% of the plurality of sequencing cycles include a sequencing solution that includes a plurality of less than four nucleotide types. In other embodiments of a method herein, greater than 50%, 60%, 70%, 80%, 90%, 95%, or 99% of the plurality of sequencing cycles include a sequencing solution that includes a plurality of less than four nucleotide types. In some embodiments of a method herein, about 50%, 60%, 70%, 80%, 90%, 95%, or 99% of the plurality of sequencing cycles include a sequencing solution that includes a plurality of less than four nucleotide types.

In embodiments of a method herein, greater than 10%, 20%, 30%, 40%, or 50% of the plurality of sequencing cycles include a sequencing solution that includes a plurality of four nucleotide types. In other embodiments of a method herein, greater than 50%, 60%, 70%, 80%, 90%, 95%, or 99% of the plurality of sequencing cycles include a sequencing solution that includes a plurality of four nucleotide types. In some embodiments of a method herein, about 50%, 60%, 70%, 80%, 90%, 95%, or 99% of the plurality of sequencing cycles include a sequencing solution that comprises a plurality of four nucleotide types.

In embodiments of a method herein, greater than 10%, 20%, 30%, 40%, or 50% of the plurality of sequencing cycles include a sequencing solution that includes a plurality of a first nucleotide type including a reversible terminator and a second nucleotide type including a reversible terminator, and two non-incorporating nucleotide types. In embodiments, greater than 50%, 60%, 70%, 80%, 90%, 95%, or 99% of the plurality of sequencing cycles include a sequencing solution that includes a plurality of a first nucleotide type including a reversible terminator and a second nucleotide type including a reversible terminator, and two non-incorporating nucleotide types. In embodiments, about 50%, 60%, 70%, 80%, 90%, 95%, or 99% of the plurality of sequencing cycles include a sequencing solution that includes a plurality of a first nucleotide type including a reversible terminator and a second nucleotide type including a reversible terminator, and two non-incorporating nucleotide types.

In embodiments of a method herein, greater than 10%, 20%, 30%, 40%, or 50% of the plurality of sequencing cycles include a sequencing solution that includes a plurality of two nucleotide types, each including a reversible terminator, and two non-incorporating nucleotide types. In other embodiments of a method herein, greater than 50%, 60%, 70%, 80%, 90%, 95%, or 99% of the plurality of sequencing cycles include a sequencing solution that includes a plurality of two nucleotide types, each including a reversible terminator, and two non-incorporating nucleotide types. In some embodiments of a method herein, about 50%, 60%, 70%, 80%, 90%, 95%, or 99% of the plurality of sequencing cycles comprise a sequencing solution that comprises a plurality of two nucleotide types, each including a reversible terminator, and two non-incorporating nucleotide types. In embodiments of a method herein, greater than 10%, 20%, 30%, 40%, or 50% of the plurality of sequencing cycles include a sequencing solution that includes a plurality of three nucleotide types, each including a reversible terminator, and one non-incorporating nucleotide type. In other embodiments of a method herein, greater than 50%, 60%, 70%, 80%, 90%, 95%, or 99% of the plurality of sequencing cycles include a sequencing solution that includes a plurality of three nucleotide types, each including a reversible terminator, and one non-incorporating nucleotide type. In some embodiments of a method herein, about 50%, 60%, 70%, 80%, 90%, 95%, or 99% of the plurality of sequencing cycles comprise a sequencing solution that comprises a plurality of three nucleotide types, each including a reversible terminator, and one non-incorporating nucleotide type. In embodiments of a method herein, greater than 10%, 20%, 30%, 40%, or 50% of the plurality of sequencing cycles include a sequencing solution that includes a plurality of one nucleotide type including a reversible terminator and three non-incorporating nucleotide type. In other embodiments of a method herein, greater than 50%, 60%, 70%, 80%, 90%, 95%, or 99% of the plurality of sequencing cycles include a sequencing solution that includes a plurality of one nucleotide type including a reversible terminator and three non-incorporating nucleotide type. In some embodiments of a method herein, about 50%, 60%, 70%, 80%, 90%, 95%, or 99% of the plurality of sequencing cycles comprise a sequencing solution that comprises a plurality of one nucleotide type including a reversible terminator and three non-incorporating nucleotide type.

In embodiments of a method herein, detecting a characteristic signature includes detecting the absence of a label. For example, when the method includes the detection of four different nucleotides using fewer than four different labels. As a first example, a pair of nucleotide types can be detected at the same wavelength, but distinguished based on a difference in signal states, such as the intensity, for one member of the pair compared to the other, or based on a change to one member of the pair (e.g., via chemical modification, photochemical modification or physical modification) that causes apparent signal to appear or disappear compared to the signal detected for the other member of the pair. As another example, three of four different nucleotide types can be detected under particular conditions while a fourth nucleotide type lacks a label that is detectable under those conditions, or is minimally detected under those conditions. Incorporation of the first three nucleotide types into a nucleic acid can be determined based on presence of their respective signals and incorporation of the fourth nucleotide type into the nucleic acid can be determined based on absence or minimal detection of any signal. As a third example, one nucleotide type can include label(s) that are detected in two different channels, whereas other nucleotide types are detected in no more than one of the channels. In some embodiments, detecting a characteristic signature comprises detecting a fluorescent emission.

In embodiments of a method herein, the reversible terminator is a 3′-reversible terminator. In other embodiments of a method herein, the reversible terminator is a virtual terminator.

In embodiments of a method herein, each nucleotide includes a 3′-reversible terminator and a detectable label. In embodiments, each non-incorporating nucleotide includes a 3′—OH. In embodiments, each non-incorporating nucleotides includes a 3′—OH and lacks a detectable label. In embodiments of a method herein, each non-incorporating nucleotide lacks a 3′-reversible terminator or a detectable label. In embodiments of a method herein, each non-incorporating nucleotide lacks a 3′-reversible terminator and a detectable label. In embodiments, the detectable label is a fluorescent dye.

In embodiments, prior to detecting the characteristic signature, the method further comprises contacting the nucleic acid templates with a dark solution, wherein the dark solution comprises a plurality of unlabeled, 3′-reversibly terminated dATPs, dCTPs, dTTPs, or dGTPs.

In embodiments, the one or more nucleotides used in the dark solution have the formula:

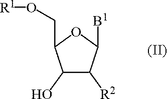

wherein R¹, R², and B¹are as described herein, including embodiments. In embodiments, four or fewer different nucleotides are present during the dark cycles and each is labeled differently. Additional compositions and methods related to dark solution and dark cycle sequencing may be found in U.S. Pat. Application No. 17/127,308, which is incorporated herein by reference in its entirety. In embodiments, B¹is

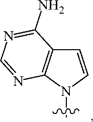

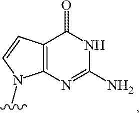

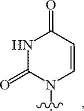

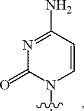

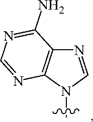

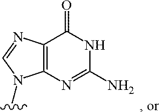

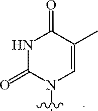

In embodiments, B¹is

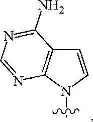

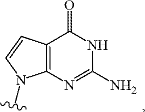

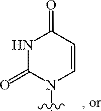

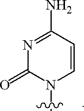

In embodiments,

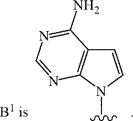

In embodiments, B¹is

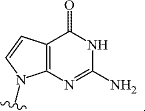

In embodiments, B¹is

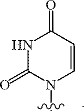

In embodiments, B¹is

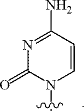

In embodiments, R²is hydrogen. In embodiments, R¹is a polyphosphate (e.g., a triphosphate).

In embodiments of a method herein, the method includes executing a plurality of sequencing cycles by contacting the nucleic acid template with a series of sequencing solutions according to a predetermined flow order. In other embodiments of a method herein, the method includes executing a plurality of sequencing cycles by contacting the nucleic acid template with a series of sequencing solutions according to a pseudorandom sequence flow order.

In some embodiments of a method herein, the predetermined flow order includes a non-cyclic binary or non-cyclic ternary sequence. In some embodiments of a method herein, the predetermined flow order includes a Thue-Morse sequence. In some embodiments, the predetermined flow order includes a de Bruijn sequence. In some embodiments, the predetermined flow order includes a Samba sequence. In some embodiments, the predetermined flow order includes a Gafieira sequence.

In embodiments, a template nucleic acid can include any nucleic acid of interest. Template nucleic acids can include DNA, RNA, peptide nucleic acid, morpholino nucleic acid, locked nucleic acid, glycol nucleic acid, threose nucleic acid, mixtures thereof, and hybrids thereof. In embodiments, the template nucleic acid is obtained from one or more source organisms. As used herein the term “organism” is not necessarily limited to a particular species of organism but can be used to refer to the living or self-replicating particle at any level of classification, which comprises the template nucleic acid. For example, the term “organism” can be used to refer collectively to all of the species within the genus Salmonella or all of the bacteria within the kingdom Eubacteria. A template nucleic acid can comprise any nucleotide sequence. In some embodiments, the template nucleic acid can include a selected sequence or a portion of a larger sequence. In embodiments, sequencing a portion of a target nucleic acid or a fragment thereof can be used to identify the source of the target nucleic acid.

In embodiments of a method herein, the template nucleic acid is about 50 to about 1500 nucleotides in length. In some embodiments of a method herein, the template nucleic acid is about 50 to about 500 nucleotides in length. In some embodiments, the template nucleic acid is greater than 100 nucleotides in length. In embodiments, the template nucleic acid is about 500 nucleotides in length. In embodiments, the template nucleic acid is about 510 nucleotides in length. In embodiments, the template nucleic acid is about 520 nucleotides in length. In embodiments, the template nucleic acid is about 530 nucleotides in length. In embodiments, the template nucleic acid is about 540 nucleotides in length. In embodiments, the template nucleic acid is about 550 nucleotides in length. In embodiments, the template nucleic acid is about 560 nucleotides in length. In embodiments, the template nucleic acid is about 570 nucleotides in length. In embodiments, the template nucleic acid is about 580 nucleotides in length. In embodiments, the template nucleic acid is about 590 nucleotides in length. In embodiments, the template nucleic acid is about 600 nucleotides in length. In embodiments, the template nucleic acid is about 610 nucleotides in length. In embodiments, the template nucleic acid is about 620 nucleotides in length. In embodiments, the template nucleic acid is about 630 nucleotides in length. In embodiments, the template nucleic acid is about 640 nucleotides in length. In embodiments, the template nucleic acid is about 650 nucleotides in length. In embodiments, the template nucleic acid is about 660 nucleotides in length. In embodiments, the template nucleic acid is about 670 nucleotides in length. In embodiments, the template nucleic acid is about 680 nucleotides in length. In embodiments, the template nucleic acid is about 690 nucleotides in length. In embodiments, the template nucleic acid is about 700 nucleotides in length. In embodiments, the template nucleic acid is about 1,200 nucleotides in length. In embodiments, the template nucleic acid is about 1,210 nucleotides in length. In embodiments, the template nucleic acid is about 1,220 nucleotides in length. In embodiments, the template nucleic acid is about 1,230 nucleotides in length. In embodiments, the template nucleic acid is about 1,240 nucleotides in length. In embodiments, the template nucleic acid is about 1,250 nucleotides in length. In embodiments, the template nucleic acid is about 1,260 nucleotides in length. In embodiments, the template nucleic acid is about 1,270 nucleotides in length. In embodiments, the template nucleic acid is about 1,280 nucleotides in length. In embodiments, the template nucleic acid is about 1,290 nucleotides in length. In embodiments, the template nucleic acid is about 1,300 nucleotides in length. In embodiments, the template nucleic acid is about 1,310 nucleotides in length. In embodiments, the template nucleic acid is about 1,320 nucleotides in length. In embodiments, the template nucleic acid is about 1,330 nucleotides in length. In embodiments, the template nucleic acid is about 1,340 nucleotides in length. In embodiments, the template nucleic acid is about 1,350 nucleotides in length. In embodiments, the template nucleic acid is about 1,360 nucleotides in length. In embodiments, the template nucleic acid is about 1,370 nucleotides in length. In embodiments, the template nucleic acid is about 1,380 nucleotides in length. In embodiments, the template nucleic acid is about 1,390 nucleotides in length. In embodiments, the template nucleic acid is about 1,400 nucleotides in length. In embodiments, the template nucleic acid is about 1,410 nucleotides in length. In embodiments, the template nucleic acid is about 1,420 nucleotides in length. In embodiments, the template nucleic acid is about 1,430 nucleotides in length. In embodiments, the template nucleic acid is about 1,440 nucleotides in length. In embodiments, the template nucleic acid is about 1,450 nucleotides in length. In embodiments, the template nucleic acid is about 1,460 nucleotides in length. In embodiments, the template nucleic acid is about 1,470 nucleotides in length. In embodiments, the template nucleic acid is about 1,480 nucleotides in length. In embodiments, the template nucleic acid is about 1,490 nucleotides in length. In embodiments, the template nucleic acid is about 1,500 nucleotides in length. In some embodiments, the template nucleic acid is greater than 1500 nucleotides in length.

In embodiments of a method herein, the sequencing read length is about 50 to about 1500 nucleotides in length. In some embodiments of a method herein, the sequencing read length is about 50 to about 500 nucleotides in length. In some embodiments, the sequencing read length is greater than 100 nucleotides in length. In embodiments, the sequencing read length is about 500 nucleotides in length. In embodiments, the sequencing read length is about 510 nucleotides in length. In embodiments, the sequencing read length is about 520 nucleotides in length. In embodiments, the sequencing read length is about 530 nucleotides in length. In embodiments, the sequencing read length is about 540 nucleotides in length. In embodiments, the sequencing read length is about 550 nucleotides in length. In embodiments, the sequencing read length is about 560 nucleotides in length. In embodiments, the sequencing read length is about 570 nucleotides in length. In embodiments, the sequencing read length is about 580 nucleotides in length. In embodiments, the sequencing read length is about 590 nucleotides in length. In embodiments, the sequencing read length is about 600 nucleotides in length. In embodiments, the sequencing read length is about 610 nucleotides in length. In embodiments, the sequencing read length is about 620 nucleotides in length. In embodiments, the sequencing read length is about 630 nucleotides in length. In embodiments, the sequencing read length is about 640 nucleotides in length. In embodiments, the sequencing read length is about 650 nucleotides in length. In embodiments, the sequencing read length is about 660 nucleotides in length. In embodiments, the sequencing read length is about 670 nucleotides in length. In embodiments, the sequencing read length is about 680 nucleotides in length. In embodiments, the sequencing read length is about 690 nucleotides in length. In embodiments, the sequencing read length is about 700 nucleotides in length. In embodiments, the sequencing read length is about 1,200 nucleotides in length. In embodiments, the sequencing read length is about 1,210 nucleotides in length. In embodiments, the sequencing read length is about 1,220 nucleotides in length. In embodiments, the sequencing read length is about 1,230 nucleotides in length. In embodiments, the sequencing read length is about 1,240 nucleotides in length. In embodiments, the sequencing read length is about 1,250 nucleotides in length. In embodiments, the sequencing read length is about 1,260 nucleotides in length. In embodiments, the sequencing read length is about 1,270 nucleotides in length. In embodiments, the sequencing read length is about 1,280 nucleotides in length. In embodiments, the sequencing read length is about 1,290 nucleotides in length. In embodiments, the sequencing read length is about 1,300 nucleotides in length. In embodiments, the sequencing read length is about 1,310 nucleotides in length. In embodiments, the sequencing read length is about 1,320 nucleotides in length. In embodiments, the sequencing read length is about 1,330 nucleotides in length. In embodiments, the sequencing read length is about 1,340 nucleotides in length. In embodiments, the sequencing read length is about 1,350 nucleotides in length. In embodiments, the sequencing read length is about 1,360 nucleotides in length. In embodiments, the sequencing read length is about 1,370 nucleotides in length. In embodiments, the sequencing read length is about 1,380 nucleotides in length. In embodiments, the sequencing read length is about 1,390 nucleotides in length. In embodiments, the sequencing read length is about 1,400 nucleotides in length. In embodiments, the sequencing read length is about 1,410 nucleotides in length. In embodiments, the sequencing read length is about 1,420 nucleotides in length. In embodiments, the sequencing read length is about 1,430 nucleotides in length. In embodiments, the sequencing read length is about 1,440 nucleotides in length. In embodiments, the sequencing read length is about 1,450 nucleotides in length. In embodiments, the sequencing read length is about 1,460 nucleotides in length. In embodiments, the sequencing read length is about 1,470 nucleotides in length. In embodiments, the sequencing read length is about 1,480 nucleotides in length. In embodiments, the sequencing read length is about 1,490 nucleotides in length. In embodiments, the sequencing read length is about 1,500 nucleotides in length. In some embodiments, the sequencing read length is greater than 1500 nucleotides in length.

In embodiments the template nucleic acid is an RNA transcript. RNA transcripts are responsible for the process of converting DNA into an organism’s phenotype, thus by determining the types and quantity of RNA present in a sample (e.g., a cell), it is possible to assign a phenotype to the cell. RNA transcripts include coding RNA and non-coding RNA molecules, such as messenger RNA (mRNA), transfer RNA (tRNA), micro RNA (miRNA), small interfering RNA (siRNA), small nucleolar RNA (snoRNA), small nuclear RNA (snRNA), Piwi-interacting RNA (piRNA), enhancer RNA (eRNA), or ribosomal RNA (rRNA). In embodiments, the template nucleic acid is pre-mRNA. In embodiments, the template nucleic acid is heterogeneous nuclear RNA (hnRNA). In embodiments the template nucleic acid is a single stranded RNA nucleic acid sequence. In embodiments, the template nucleic acid is an RNA nucleic acid sequence or a DNA nucleic acid sequence (e.g., cDNA). In embodiments, the template nucleic acid is a cDNA target nucleic acid sequence. In embodiments, the template nucleic acid is genomic DNA (gDNA), mitochondrial DNA, chloroplast DNA, episomal DNA, viral DNA, or complementary DNA (cDNA). In embodiments, the template nucleic acid is coding RNA such as messenger RNA (mRNA), and non-coding RNA (ncRNA) such as transfer RNA (tRNA), microRNA (miRNA), small nuclear RNA (snRNA), or ribosomal RNA (rRNA).

In embodiments, the template nucleic acids are RNA nucleic acid sequences or DNA nucleic acid sequences. In embodiments, the template nucleic acids are RNA nucleic acid sequences or DNA nucleic acid sequences from the same cell. In embodiments, the template nucleic acids are RNA nucleic acid sequences. In embodiments, the RNA nucleic acid sequence is stabilized using known techniques in the art. For example, RNA degradation by RNase should be minimized using commercially available solutions (e.g., RNA Later®, RNA Protect®, or DNA/RNA Shield®). In embodiments, the sample polynucleotides are messenger RNA (mRNA), transfer RNA (tRNA), micro RNA (miRNA), small interfering RNA (siRNA), small nucleolar RNA (snoRNA), small nuclear RNA (snRNA), Piwi-interacting RNA (piRNA), enhancer RNA (eRNA), or ribosomal RNA (rRNA). In embodiments, the template nucleic acid is pre-mRNA. In embodiments, the template nucleic acid is heterogeneous nuclear RNA (hnRNA). In embodiments, the template nucleic acid is mRNA, tRNA (transfer RNA), rRNA (ribosomal RNA), or noncoding RNA (such as lncRNA (long noncoding RNA)). In embodiments, the template nucleic acids are on different regions of the same RNA nucleic acid sequence. In embodiments, the template nucleic acid is cDNA target nucleic acid sequences and before step i), the RNA nucleic acid sequences are reverse transcribed to generate the cDNA target nucleic acid sequences. In embodiments, the template nucleic acid is not reverse transcribed to cDNA. When mRNA is reverse transcribed an oligo(dT) primer can be added to better hybridize to the poly A tail of the mRNA. The oligo(dT) primer may include between about 12 and about 25 dT residues. The oligo(dT) primer may be an oligo(dT) primer of between about 18 to about 25 nt in length.

In embodiments of a method herein, at least 10 to 200 nucleotides are incorporated into the sequencing primer. In embodiments, about 10 nucleotides are incorporated into the sequencing primer. In embodiments, about 20 nucleotides are incorporated into the sequencing primer. In embodiments, about 30 nucleotides are incorporated into the sequencing primer. In embodiments, about 40 nucleotides are incorporated into the sequencing primer. In embodiments, about 50 nucleotides are incorporated into the sequencing primer. In embodiments, about 60 nucleotides are incorporated into the sequencing primer. In embodiments, about 70 nucleotides are incorporated into the sequencing primer. In embodiments, about 80 nucleotides are incorporated into the sequencing primer. In embodiments, about 90 nucleotides are incorporated into the sequencing primer. In some embodiments, about 100 to 1000 nucleotides are incorporated into the sequencing primer. In other embodiments, about 100 to 500 nucleotides are incorporated into the sequencing primer. In other embodiments, greater than 200 nucleotides are incorporated into the sequencing primer. In embodiments, about 100 nucleotides are incorporated into the sequencing primer. In embodiments, about 200 nucleotides are incorporated into the sequencing primer. In embodiments, about 300 nucleotides are incorporated into the sequencing primer. In embodiments, about 400 nucleotides are incorporated into the sequencing primer. In embodiments, about 500 nucleotides are incorporated into the sequencing primer. In embodiments, about 600 nucleotides are incorporated into the sequencing primer. In embodiments, about 700 nucleotides are incorporated into the sequencing primer. In embodiments, about 800 nucleotides are incorporated into the sequencing primer. In embodiments, about 900 nucleotides are incorporated into the sequencing primer. In embodiments, about 1,000 nucleotides are incorporated into the sequencing primer.

In embodiments of a method herein, each sequencing cycle includes a probability of an incorrect base call that is less than 1 in 100. In some embodiments, each sequencing cycle includes a probability of an incorrect base call that is less than 1 in 1,000. In some embodiments, each sequencing cycle includes a probability of an incorrect base call that is less than 1 in 10,000.

In embodiments of a method herein, greater than 85% of the templates are in phase following each sequencing cycle. In embodiments, greater than 90% of the templates are in phase following each sequencing cycle. In embodiments, greater than 91% of the templates are in phase following each sequencing cycle. In embodiments, greater than 92% of the templates are in phase following each sequencing cycle. In embodiments, greater than 93% of the templates are in phase following each sequencing cycle. In embodiments, greater than 94% of the templates are in phase following each sequencing cycle. In embodiments, greater than 95% of the templates are in phase following each sequencing cycle. In embodiments, greater than 96% of the templates are in phase following each sequencing cycle. In embodiments, greater than 97% of the templates are in phase following each sequencing cycle. In embodiments, greater than 98% of the templates are in phase following each sequencing cycle. In embodiments, greater than 99% of the templates are in phase following each sequencing cycle. In embodiments, greater than 99.9% of the templates are in phase following each sequencing cycle. In embodiments, greater than 80% of the templates are in phase after 50 sequencing cycles. In embodiments greater than 60% of templates are in phase after 100 sequencing cycles. The percentage of templates in phase represents the average fraction of in-phase templates among clusters analyzed in a sequencing run.

In embodiments of a method herein, each sequencing cycle includes a probability of an incorrect base call that is less than 1 in 100 for about 200 to 1,000 nucleotide incorporations. In some embodiments, each sequencing cycle includes a probability of an incorrect base call that is less than 1 in 1,000 for about 200 to 1,000 nucleotide incorporations. In some embodiments, each sequencing cycle includes a probability of an incorrect base call that is less than 1 in 10,000 for about 200 to 1,000 nucleotide incorporations. In other embodiments, each sequencing cycle includes a probability of an incorrect base call that is less than 1 in 100 for about 300 to 1,000 nucleotide incorporations. In some embodiments, each sequencing cycle includes a probability of an incorrect base call that is less than 1 in 1,000 for about 300 to 1,000 nucleotide incorporations. In some embodiments, each sequencing cycle includes a probability of an incorrect base call that is less than 1 in 10,000 for about 300 to 1,000 nucleotide incorporations. In other embodiments, each sequencing cycle includes a probability of an incorrect base call that is less than 1 in 100 for about 500 to 1,000 nucleotide incorporations. In some embodiments, each sequencing cycle includes a probability of an incorrect base call that is less than 1 in 1,000 for about 500 to 1,000 nucleotide incorporations. In some embodiments, each sequencing cycle includes a probability of an incorrect base call that is less than 1 in 10,000 for about 500 to 1,000 nucleotide incorporations. In other embodiments, each sequencing cycle includes a probability of an incorrect base call that is less than 1 in 100 for about 750 to 1,000 nucleotide incorporations. In some embodiments, each sequencing cycle includes a probability of an incorrect base call that is less than 1 in 1,000 for about 750 to 1,000 nucleotide incorporations. In some embodiments, each sequencing cycle includes a probability of an incorrect base call that is less than 1 in 10,000 for about 750 to 1,000 nucleotide incorporations. In other embodiments, each sequencing cycle includes a probability of an incorrect base call that is less than 1 in 100 for about 900 to 1,000 nucleotide incorporations. In some embodiments, each sequencing cycle includes a probability of an incorrect base call that is less than 1 in 1,000 for about 900 to 1,000 nucleotide incorporations. In some embodiments, each sequencing cycle includes a probability of an incorrect base call that is less than 1 in 10,000 for about 900 to 1,000 nucleotide incorporations.

In an aspect is a method of sequencing a nucleic acid template, the method including hybridizing one or more sequencing primers to a nucleic acid template; executing a plurality of sequencing cycles, each cycle including contacting the nucleic acid template with the first and second sequencing mixtures of a kit of the invention, and embodiments herein, in the presence of a polymerase; and detecting a characteristic signature indicating that a nucleotide from the first or second sequencing mixtures has been incorporated into the sequencing primer.

In embodiments, the template nucleic acid includes a gene or a gene fragment. In embodiments, the gene or gene fragment is a cancer-associated gene or fragment thereof, T cell receptor (TCRs) gene or fragment thereof, or a B cell receptor (BCRs) gene, or fragment thereof. In embodiments, the gene or gene fragment is a CDR3 gene or fragment thereof. In embodiments, the gene or gene fragment is a T cell receptor alpha variable (TRAV) gene or fragment thereof, T cell receptor alpha joining (TRAJ) gene or fragment thereof, T cell receptor alpha constant (TRAC) gene or fragment thereof, T cell receptor beta variable (TRBV) gene or fragment thereof, T cell receptor beta diversity (TRBD) gene or fragment thereof, T cell receptor beta joining (TRBJ) gene or fragment thereof, T cell receptor beta constant (TRBC) gene or fragment thereof, T cell receptor gamma variable (TRGV) gene or fragment thereof, T cell receptor gamma joining (TRGJ) gene or fragment thereof, T cell receptor gamma constant (TRGC) gene or fragment thereof, T cell receptor delta variable (TRDV) gene or fragment thereof, T cell receptor delta diversity (TRDD) gene or fragment thereof, T cell receptor delta joining (TRDJ) gene or fragment thereof, or T cell receptor delta constant (TRDC) gene or fragment thereof. In embodiments, the polynucleotide includes genomic DNA, complementary DNA (cDNA), cell-free DNA (cfDNA), messenger RNA (mRNA), transfer RNA (tRNA), ribosomal RNA (rRNA), cell-free RNA (cfRNA), or noncoding RNA (ncRNA). In embodiments, the polynucleotide includes messenger RNA (mRNA), transfer RNA (tRNA), micro RNA (miRNA), small interfering RNA (siRNA), small nucleolar RNA (snoRNA), small nuclear RNA (snRNA), Piwi-interacting RNA (piRNA), enhancer RNA (eRNA), or ribosomal RNA (rRNA).

In embodiments, the template nucleic acid includes a gene fusion. Gene fusions are a type of somatic alteration leading to cancer associated with up to 20% of cancer morbidity and having oncogenic roles in hematological, soft tissue, and solid tumors (Foltz SM et al. Nature Comm. 2020; 11:2666). Translocations, copy number changes, and inversions can lead to fusions, dysregulared gene expression, and novel molecular functions. In embodiments, the gene fusion includes a CD74-ROS1, SLC34A2-ROS1, SDC4-ROS1, EZR-ROS1, GOPC-ROS1, LRIG3-ROS1, TPM3-ROS1, PPFIBP1-ROS1, EML4-ALK, BCR-ABL, TCF3-PBX1, ETV6-RUNX1, MLL-AF4, SIL-TAL1, RET-NTRK1, PAX8-PPARG, MECT1-MAML2, TFE3-TFEB, BRD4-NUT, ETV6-NTRK3, TMPRSS2-ERG, TPM3-NTRK1, SQSTM1-NTRK1, CD74-NTRK1, MPRIP-NTRK1, or TRIM24-NTRK2, wherein the gene fusion is written in the format [gene1]-[gene2]. In embodiments, the gene fusion includes a ROS1 gene or fragment thereof, ALK gene or fragment thereof, EML4 gene or fragment thereof, BCR gene or fragment thereof, ABL gene or fragment thereof, TCF3 gene or fragment thereof, PBX1 gene or fragment thereof, ETV6 gene or fragment thereof, RUNX1 gene or fragment thereof, MLL gene or fragment thereof, AF4 gene or fragment thereof, SIL gene or fragment thereof, TAL1 gene or fragment thereof, RET gene or fragment thereof, NTRK1 gene or fragment thereof, PAX8 gene or fragment thereof, PPARG gene or fragment thereof, MECT1 gene or fragment thereof, MAML2 gene or fragment thereof, TFE3 gene or fragment thereof, TFEB gene or fragment thereof, BRD4 gene or fragment thereof, NUT gene or fragment thereof, ETV6 gene or fragment thereof, NTRK3 gene or fragment thereof, TMPRSS2 gene or fragment thereof, NKRT2 gene or fragment thereof, an ERG gene or fragment thereof, and at least one other gene.

In embodiments, the methods and compositions described herein are utilized to analyze the various sequences of T cell receptors (TCRs) and B cell receptors (BCRs) from immune cells, for example various clonotypes. In embodiments, the target nucleic acid includes a nucleic acid sequence encoding a TCR alpha (TCRA) chain, a TCR beta (TCRB) chain, a TCR delta (TCRD) chain, a TCR gamma (TCRG) chain, or any fragment thereof (e.g., variable regions including VDJ or VJ regions, constant regions, transmembrane regions, fragments thereof, combinations thereof, and combinations of fragments thereof). In embodiments, the template nucleic acid includes a nucleic acid sequence encoding a B cell receptor heavy chain, B cell receptor light chain, or any fragment thereof (e.g., variable regions including VDJ or VJ regions, constant regions, transmembrane regions, fragments thereof, combinations thereof, and combinations of fragments thereof). In embodiments, the template nucleic acid includes a CDR3 nucleic acid sequence. In embodiments, the template nucleic acid includes a TCRA gene sequence or a TCRB gene sequence. In embodiments, the template nucleic acid includes a TCRA gene sequence and a TCRB gene sequence. In embodiments, the template nucleic acid includes sequences of various T cell receptor alpha variable genes (TRAV genes), T cell receptor alpha joining genes (TRAJ genes), T cell receptor alpha constant genes (TRAC genes), T cell receptor beta variable genes (TRBV genes), T cell receptor beta diversity genes (TRBD genes), T cell receptor beta joining genes (TRBJ genes), T cell receptor beta constant genes (TRBC genes), T cell receptor gamma variable genes (TRGV genes), T cell receptor gamma joining genes (TRGJ genes), T cell receptor gamma constant genes (TRGC genes), T cell receptor delta variable genes (TRDV genes), T cell receptor delta diversity genes (TRDD genes), T cell receptor delta joining genes (TRDJ genes), or T cell receptor delta constant genes (TRDC genes).

In embodiments, the methods described herein can utilize a single template nucleic acid. Other embodiments can utilize a plurality of template nucleic acids. In such embodiments, a plurality of template nucleic acids can include a plurality of the same template nucleic acids, a plurality of different template nucleic acids where some template nucleic acids are the same, or a plurality of template nucleic acids where all template nucleic acids are different. In some embodiments, the plurality of template nucleic acids can include substantially all of a particular organism’s genome. In some embodiments, the plurality of template nucleic acids can include at least a portion of a particular organism’s genome including, for example, at least about 10%, 25%, 50%, 75%, 80%, 85%, 90%, 95%, or 99% of the genome. In other embodiments, the plurality of template nucleic acids can include a single nucleotide sequence of the genome of an organism or a single expressed nucleotide sequence. In still other embodiments, the plurality of template nucleic acids can include a portion of a single nucleotide sequence of the genome of an organism or a portion of a single expressed nucleotide sequence. With reference to nucleic acids, polynucleotides and/or nucleotide sequences a “portion,” “fragment” or “region” can be at least 5 consecutive nucleotides, at least 10 consecutive nucleotides, at least 15 consecutive nucleotides, at least 20 consecutive nucleotides, at least 25 consecutive nucleotides, at least 50 consecutive nucleotides or at least 100 consecutive nucleotides.

In embodiments, the methods of sequencing a template nucleic acid include a extending a polynucleotide by using a polymerase. In embodiments, the polymerase is a DNA polymerase. In embodiments, the DNA polymerase is a Pol I DNA polymerase, Pol II DNA polymerase, Pol III DNA polymerase, Pol IV DNA polymerase, Pol V DNA polymerase, Pol β DNA polymerase, Pol µ DNA polymerase, Pol λ DNA polymerase, Pol σ DNA polymerase, Pol α DNA polymerase, Pol δ DNA polymerase, Pol ε DNA polymerase, Pol η DNA polymerase, Pol v DNA polymerase, Pol κ DNA polymerase, Pol ζ DNA polymerase, Pol γ DNA polymerase, Pol θ DNA polymerase, Pol υ DNA polymerase, or a thermophilic nucleic acid polymerase (e.g. Therminator γ, 9°N polymerase (exo-), Therminator II, Therminator III, or Therminator IX). In embodiments, the DNA polymerase is a thermophilic nucleic acid polymerase. In embodiments, the DNA polymerase is a modified archaeal DNA polymerase. In embodiments, the polymerase is a bacterial DNA polymerase, eukaryotic DNA polymerase, archaeal DNA polymerase, viral DNA polymerase, or phage DNA polymerases. Bacterial DNA polymerases include E. coli DNA polymerases I, II and III, IV and V, the Klenow fragment of E. coli DNA polymerase, Clostridium stercorarium (Cst) DNA polymerase, Clostridium thermocellum (Cth) DNA polymerase and Sulfolobus solfataricus (Sso) DNA polymerase. Eukaryotic DNA polymerases include DNA polymerases α, β, γ, δ, €, η, ζ, λ, σ, µ, and k, as well as the Revl polymerase (terminal deoxycytidyl transferase) and terminal deoxynucleotidyl transferase (TdT). Viral DNA polymerases include T4 DNA polymerase, phi-29 DNA polymerase, GA-1, phi-29-like DNA polymerases, PZA DNA polymerase, phi-15 DNA polymerase, Cpl DNA polymerase, Cpl DNA polymerase, T7 DNA polymerase, and T4 polymerase. Other useful DNA polymerases include thermostable and/or thermophilic DNA polymerases such as Thermus aquaticus (Taq) DNA polymerase, Thermus filiformis (Tfi) DNA polymerase, Thermococcus zilligi (Tzi) DNA polymerase, Thermus thermophilus (Tth) DNA polymerase, Thermus flavusu (Tfl) DNA polymerase, Pyrococcus woesei (Pwo) DNA polymerase, Pyrococcus furiosus (Pfu) DNA polymerase and Turbo Pfu DNA polymerase, Thermococcus litoralis (Tli) DNA polymerase, Pyrococcus sp. GB-D polymerase, Thermotoga maritima (Tma) DNA polymerase, Bacillus stearothermophilus (Bst) DNA polymerase, Pyrococcus Kodakaraensis (KOD) DNA polymerase, Pfx DNA polymerase, Thermococcus sp. JDF-3 (JDF-3) DNA polymerase, Thermococcus gorgonarius (Tgo) DNA polymerase, Thermococcus acidophilium DNA polymerase; Sulfolobus acidocaldarius DNA polymerase; Thermococcus sp. go N-7 DNA polymerase; Pyrodictium occultum DNA polymerase; Methanococcus voltae DNA polymerase; Methanococcus thermoautotrophicum DNA polymerase; Methanococcus jannaschii DNA polymerase; Desulfurococcus strain TOK DNA polymerase (D. Tok Pol); Pyrococcus abyssi DNA polymerase; Pyrococcus horikoshii DNA polymerase; Pyrococcus islandicum DNA polymerase; Thermococcus fumicolans DNA polymerase; Aeropyrum pemix DNA polymerase; and the heterodimeric DNA polymerase DP1/DP2. In embodiments, the polymerase is 3PDX polymerase as disclosed in U.S. 8,703,461, the disclosure of which is incorporated herein by reference. In embodiments, the polymerase is a reverse transcriptase. Exemplary reverse transcriptases include, but are not limited to, HIV-1 reverse transcriptase from human immunodeficiency virus type 1 (PDB 1HMV), HIV-2 reverse transcriptase from human immunodeficiency virus type 2, M-MLV reverse transcriptase from the Moloney murine leukemia virus, AMV reverse transcriptase from the avian myeloblastosis virus, or Telomerase reverse transcriptase. In embodiments, the polymerase is a reverse transcriptase. In embodiments, the polymerase is a mutant P. abyssi polymerase (e.g., such as a mutant P. abyssi polymerase described in WO 2018/148723 or WO 2020/056044, both of which are incorporated by reference herein). In embodiments, the polymerase is DNA polymerase, a terminal deoxynucleotidyl transferase, or a reverse transcriptase. In embodiments, the enzyme is a DNA polymerase, such as DNA polymerase 812 (Pol 812) or DNA polymerase 1901 (Pol 1901), e.g., a polymerase described in US 2020/0131484, and US 2020/0181587, both of which are incorporated by reference herein.

In embodiments, the methods of sequencing a template nucleic acid include extending a complementary polynucleotide that is hybridized to the template nucleic acid by incorporating a first nucleotide. In embodiments, the nucleotide is selected from one or more of dATP, dCTP, dGTP, and dTTP or an analogue thereof. In embodiments, the nucleotide includes a detectable label. In embodiments, the detectable label is a fluorescent label. In embodiments, the nucleotide includes a reversible terminator moiety. In embodiments, the reversible terminator moiety may be 3′—O—blocked reversible terminator. In nucleotides with 3′—O—blocked reversible terminators, the blocking group (referred to as -OR) wherein the O of -OR is the oxygen atom of the 3′—OH of the pentose, and R of -OR is the blocking group (i.e. the reversible terminator moiety) while the label is linked to the base, which acts as a reporter and can be cleaved. The 3′—O—blocked reversible terminators are known in the art, and may be, for instance, a 3′-ONH₂reversible terminator, a 3′—O—allyl reversible terminator, or a 3′—O—azidomethyl reversible terminator. In embodiments, the reversible terminator moiety is

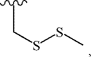

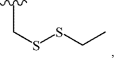

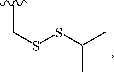

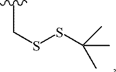

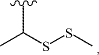

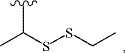

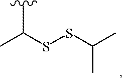

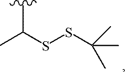

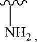

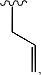

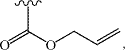

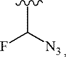

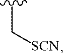

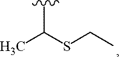

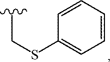

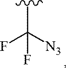

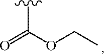

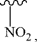

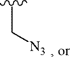

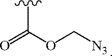

In embodiments, the method comprises a plurality of cycles, with each cycle comprising incorporation and identification of a first nucleotide. In some embodiments of methods comprising a plurality of sequencing cycles, the first nucleotide incorporated in one cycle of the plurality of cycles may be the same or different from the first nucleotide incorporated in another cycle of the plurality of cycles.

In embodiments, the nucleotide has the formula:

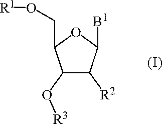

, wherein B¹ is a nucleobase (e.g., a nucleobase including a covalent linker optionally bonded to a detectable moiety, for example as described herein). In embodiments, B¹is a substituted or unsubstituted nucleobase (e.g., -B-L¹⁰⁰-R⁴); R¹is —OH, a monophosphate moiety, or polyphosphate moiety (e.g., triphosphate); R²is —OH or hydrogen; and R³is a reversible terminator moiety. In embodiments, R²is hydrogen.

In embodiments, B¹is

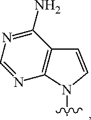

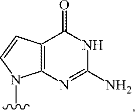

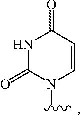

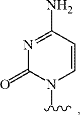

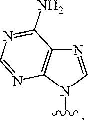

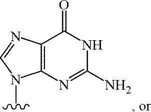

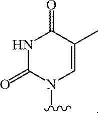

In embodiments, B¹is

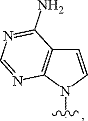

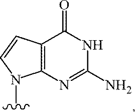

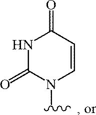

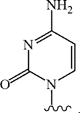

In embodiments, B¹is

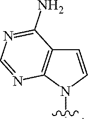

In embodiments, B¹is

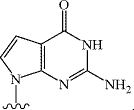

In embodiments, B¹is

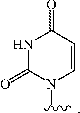

In embodiments, B¹

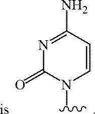

In embodiments, B¹is

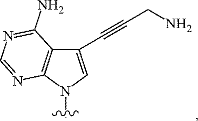

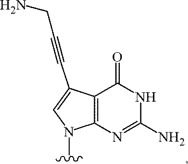

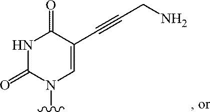

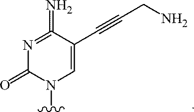

In embodiments, B¹is

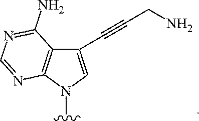

In embodiments, B¹is

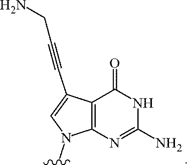

In embodiments, B¹

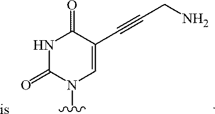

In embodiments, B¹is

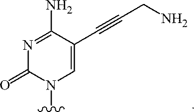

In embodiments, B¹ is

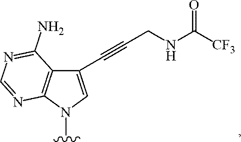

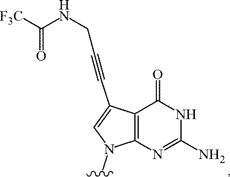

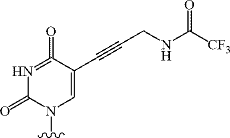

or

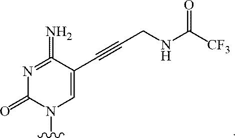

In embodiments, B¹is

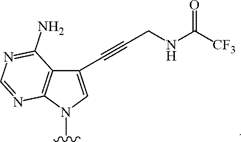

In embodiments, B¹is

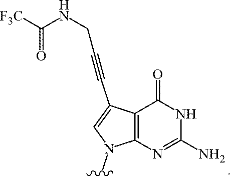

In embodiments, B¹is

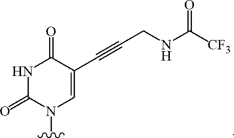

In embodiments, B¹is

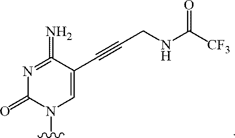

In embodiments, B¹is -B-L¹⁰⁰-R⁴; wherein B is a divalent nucleobase, L¹⁰⁰is a divalent linker, and R⁴is a detectable moiety B is a divalent cytosine or a derivative thereof, divalent guanine or a derivative thereof, divalent adenine or a derivative thereof, divalent thymine or a derivative thereof, divalent uracil or a derivative thereof, divalent hypoxanthine or a derivative thereof, divalent xanthine or a derivative thereof, divalent 7-methylguanine or a derivative thereof, divalent 5,6-dihydrouracil or a derivative thereof, divalent 5-methylcytosine or a derivative thereof, or divalent 5-hydroxymethylcytosine or a derivative thereof. L¹⁰⁰is a divalent linker; and R⁴ is a detectable moiety. In embodiments, L¹⁰⁰is independently a bioconjugate linker, a cleavable linker, or a self-immolative linker. In embodiments, B¹is a divalent nucleobase. In embodiments, B¹is

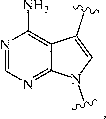

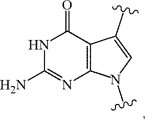

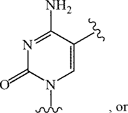

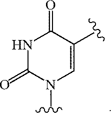

In embodiments, B¹is

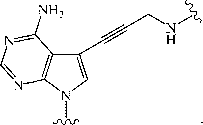

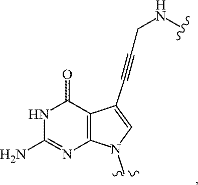

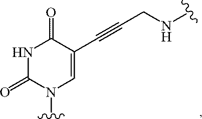

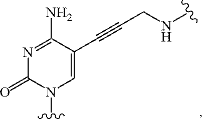

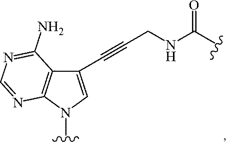

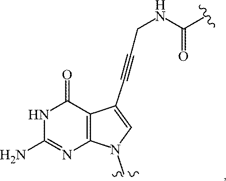

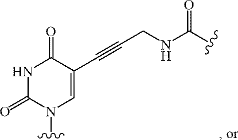

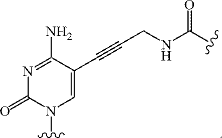

In embodiments, L¹⁰⁰is

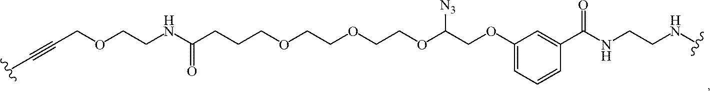

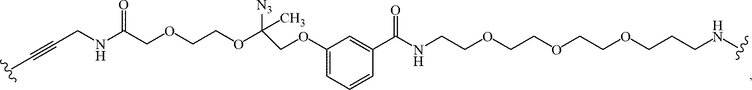

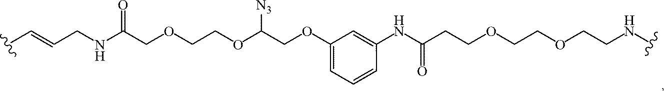

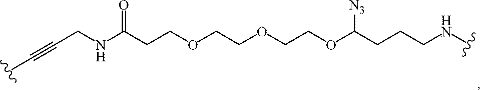

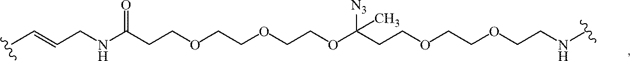

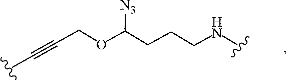

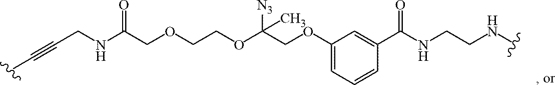

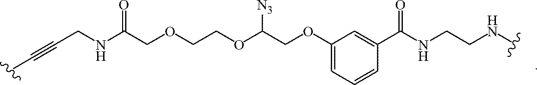

In embodiments, L¹⁰⁰is

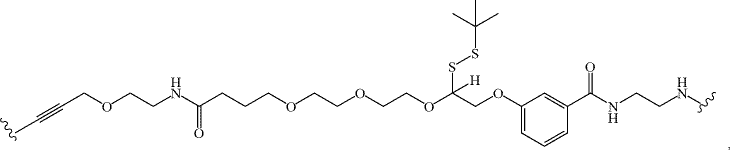

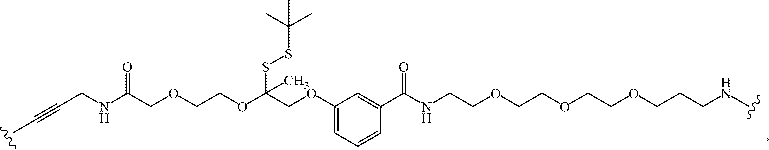

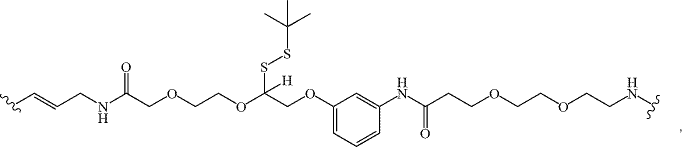

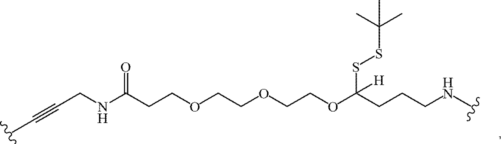

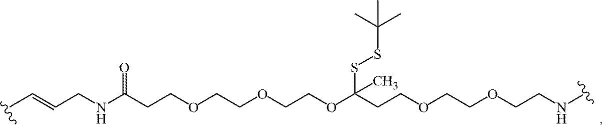

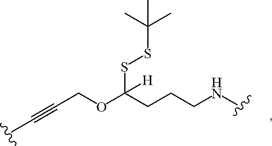

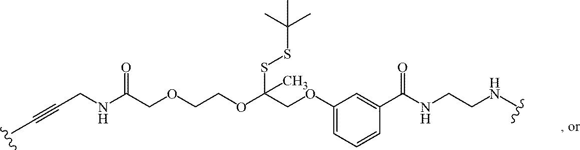

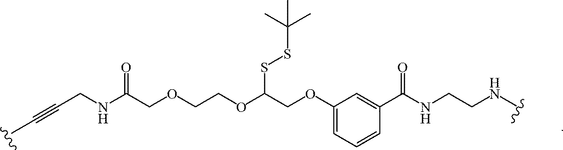

In embodiments, L¹⁰⁰is

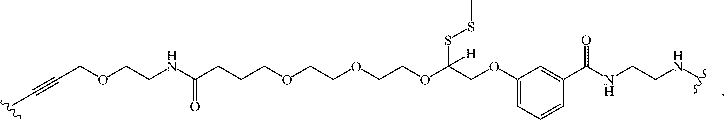

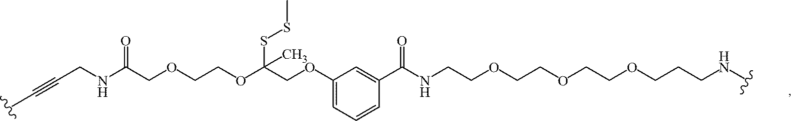

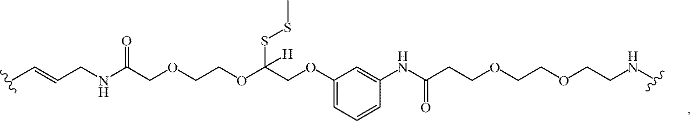

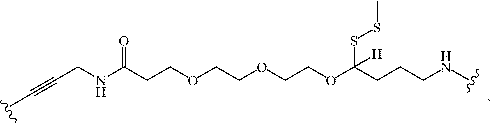

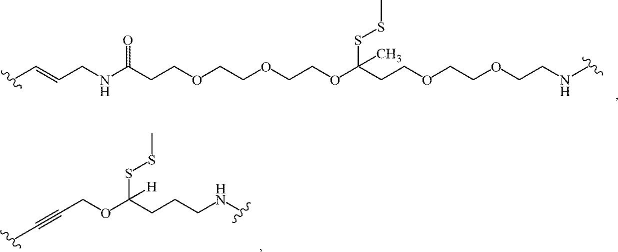

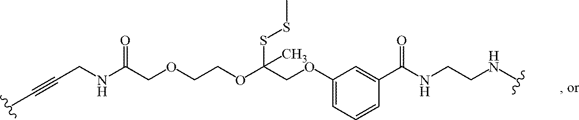

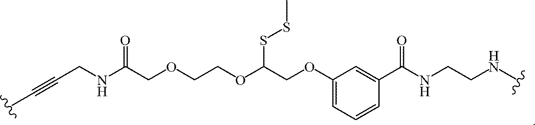

In embodiments, L¹⁰⁰is

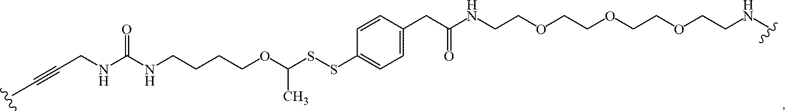

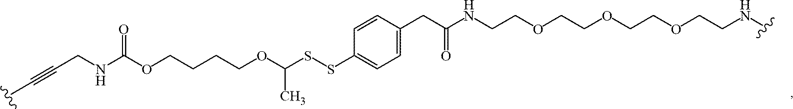

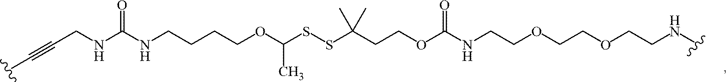

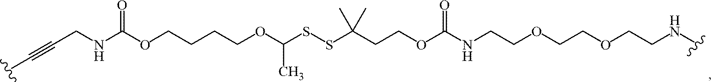

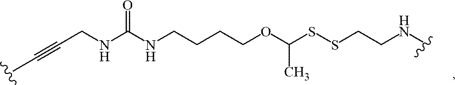

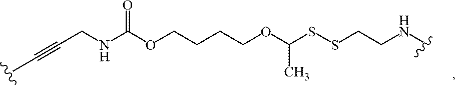

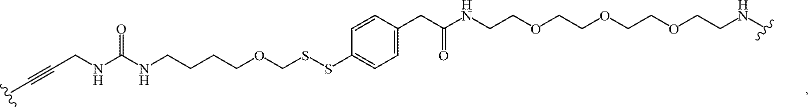

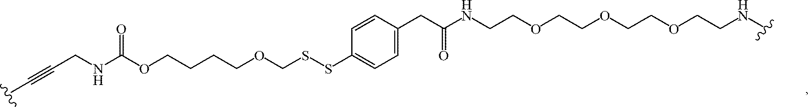

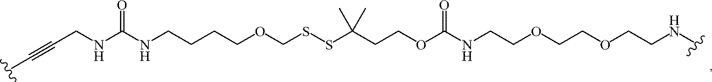

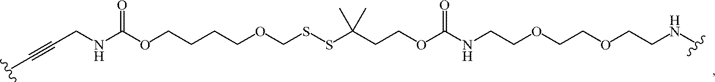

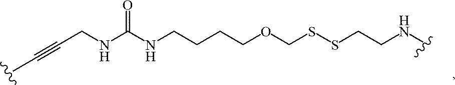

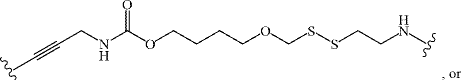

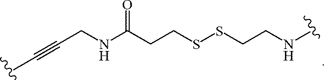

In embodiments, R³ is

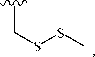

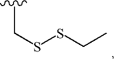

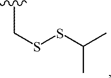

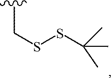

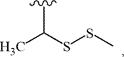

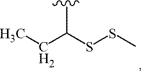

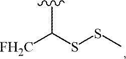

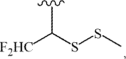

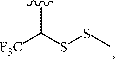

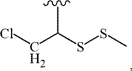

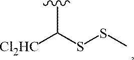

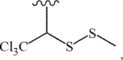

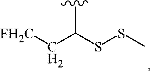

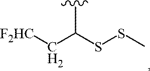

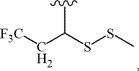

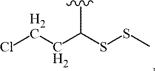

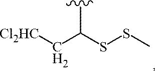

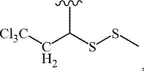

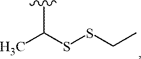

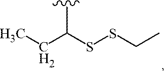

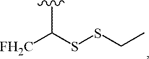

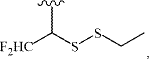

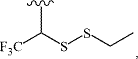

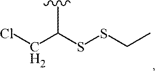

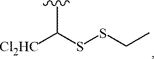

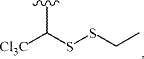

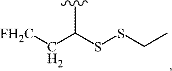

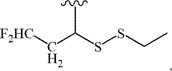

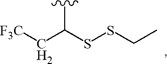

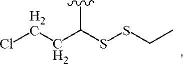

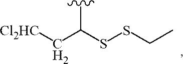

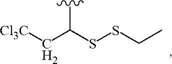

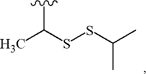

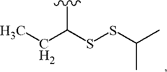

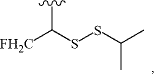

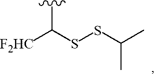

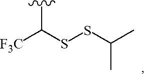

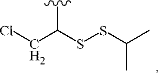

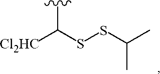

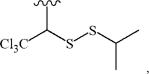

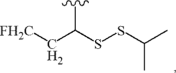

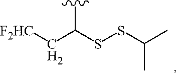

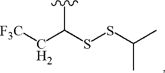

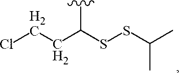

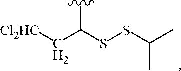

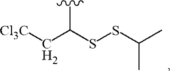

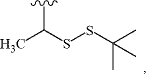

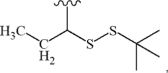

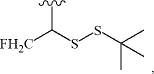

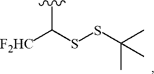

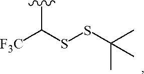

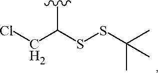

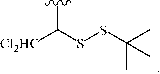

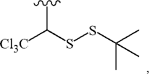

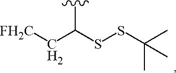

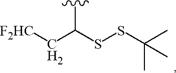

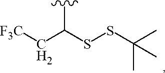

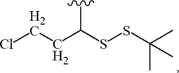

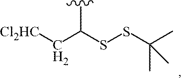

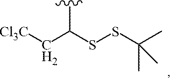

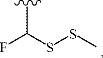

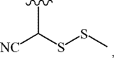

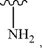

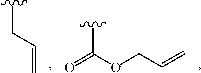

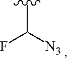

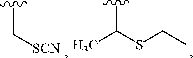

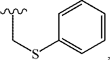

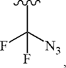

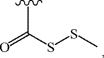

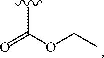

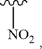

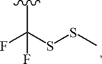

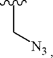

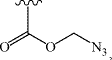

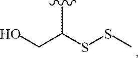

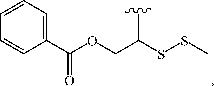

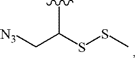

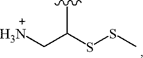

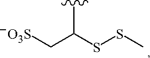

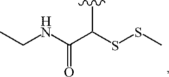

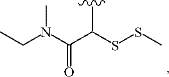

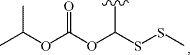

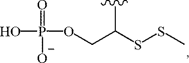

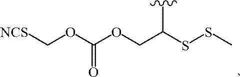

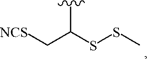

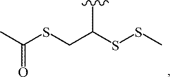

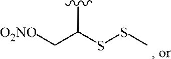

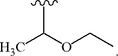

In embodiments, R⁴is a detectable moiety. In embodiments, R⁴is a fluorescent dye moiety. In embodiments, R⁴is a detectable moiety described herein (e.g., Dye Table). In embodiments, R⁴is a detectable moiety described in the Dye Table.

Dye Table: Detectable moieties to be used in selected embodiments.

| Nucleoside/nucleotide abbreviation | Dye name | λmax (nm) |
|---|---|---|
| dC | Atto 532 | 532 |
| dC | Atto Rho 6G | 535 |
| dC | R6G | 534 |
| dC | Tet | 521 |
| dT | Atto Rho 11 | 572 |
| dT | Atto 565 | 564 |
| dT | Alexa Fluor 568 | 578 |
| dT | dTamra | 578 |
| dA | Alexa Fluor 647 | 650 |
| dA | Atto 647N | 644 |
| dA | Janelia Fluor 646 | 646 |
| dG | Alexa Fluor 680 | 682 |
| dG | Alexa Fluor 700 | 696 |
| dG | CF680R | 680 |

In embodiments, the methods of sequencing a template nucleic acid further include aligning the one or more sequencing reads to a reference sequence. General methods for performing sequence alignments are known to those skilled in the art. Examples of suitable alignment algorithms, include but not limited to the Needleman-Wunsch algorithm (see e.g. the EMBOSS Needle aligner available at www.ebi.ac.uk/Tools/psa/emboss_needle/, optionally with default settings), the BLAST algorithm (see e.g. the BLAST alignment tool available at blast.ncbi.nlm.nih.gov/Blast.cgi, optionally with default settings), or the Smith-Waterman algorithm (see e.g. the EMBOSS Water aligner available at www.ebi.ac.uk/Tools/psa/emboss_water/, optionally with default settings). Optimal alignment may be assessed using any suitable parameters of a chosen algorithm, including default parameters.

In embodiments, the methods of sequencing a template nucleic acid further include generating overlapping sequence reads and assembling them into a contiguous nucleotide sequence of a nucleic acid of interest. Assembly algorithms known in the art can align and merge overlapping sequence reads generated by methods of several embodiments herein to provide a contiguous sequence of a nucleic acid of interest. A person of ordinary skill in the art will understand which sequence assembly algorithms or sequence assemblers are suitable for a particular purpose taking into account the type and complexity of the nucleic acid of interest to be sequenced (e.g. genomic, PCR product, or plasmid), the number and/or length of deletion products or other overlapping regions generated, the type of sequencing methodology performed, the read lengths generated, whether assembly is de novo assembly of a previously unknown sequence or mapping assembly against a backbone sequence, etc. Furthermore, an appropriate data analysis tool will be selected based on the function desired, such as alignment of sequence reads, base-calling and/or polymorphism detection, de novo assembly, assembly from paired or unpaired reads, and genome browsing and annotation. In several embodiments, overlapping sequence reads can be assembled by sequence assemblers, including but not limited to ABySS, AMOS, Arachne WGA, CAP3, PCAP, Celera WGA Assembler/CABOG, CLC Genomics Workbench, CodonCode Aligner, Euler, Euler-sr, Forge, Geneious, MIRA, miraEST, NextGENe, Newbler, Phrap, TIGR Assembler, Sequencher, SeqMan NGen, SHARCGS, SSAKE, Staden gap4 package, VCAKE, Phusion assembler, Quality Value Guided SRA (QSRA), SPAdes,Velvet (algorithm), and the like.

It will be understood that overlapping sequence reads can also be assembled into contigs or the full contiguous sequence of the nucleic acid of interest by available means of sequence alignment, computationally or manually, whether by pairwise alignment or multiple sequence alignment of overlapping sequence reads. Algorithms suited for short-read sequence data may be used in a variety of embodiments, including but not limited to Cross match, ELAND, Exonerate, MAQ, Mosaik, RMAP, SHRiMP, SOAP, SSAHA2, SXOligoSearch, ALLPATHS, Edena, Euler-SR, SHARCGS, SHRAP, SSAKE, VCAKE, SPAdes, Velvet, PyroBayes, PbShort, and ssahaSNP.

In embodiments, the methods of sequencing a template nucleic acid further include generating a consensus sequence for the template nucleic acid and/or its complement from the alignment of one or more sequencing reads. Multiple sequencing reads spanning the same region but with different start and stop positions for sequencing and dark cycles can be collapsed into a consensus sequence that combines sequencing information from the various sequencing cycles.

In embodiments, the methods of sequencing a template nucleic acid provide additional information about the substitution error rate. In embodiments, the methods of sequencing a template nucleic acid provide additional information about the indel error rate. In embodiments, the methods of sequencing template nucleic acids provide lower substitution error rates than indel error rates. In embodiments, the methods of sequencing a template nucleic acid provide sequencing reads with reduced indel error rates relative to traditional sequencing flow orders. In embodiments, the methods of sequencing a template nucleic acid provide sequencing reads with reduced substitution error rates relative to traditional sequencing flow orders.

A variety of suitable sequencing platforms are available for implementing methods disclosed herein. Non-limiting examples include SMRT (single-molecule real-time sequencing), ion semiconductor, pyrosequencing, sequencing by synthesis, combinatorial probe anchor synthesis, SOLiD sequencing (sequencing by ligation), and nanopore sequencing. Sequencing platforms include those provided by Illumina® (e.g., the HiSeq™, MiSeq™and/or Genome Analyzer™sequencing systems); Ion Torrent™(e.g., the Ion PGM™and/or Ion Proton™. sequencing systems); Pacific Biosciences (e.g., the PACBIO RS II sequencing system); Life Technologies™(e.g., a SOLiD sequencing system); Roche (e.g., the 454 GS FLX+ and/or GS Junior sequencing systems). See, for example U.S Pat. 7,211,390; U.S Pat. 7,244,559; U.S Pat. 7,264,929; US Patent 6,255,475; US patent 6,013,445; U.S Pat. 8,882,980; U.S Pat. 6,664,079; and U.S Pat. 9,416,409. Useful pyrosequencing reactions are described, for example, in U.S Pat. Application Publication No. 2005/0191698 and U.S. Pat. No. 7,244,559, each of which is incorporated herein by reference. Sequencing-by-ligation reactions are described, for example, in Shendure et al. Science 309:1728-1732 (2005); U.S. Pat. Nos. 5,599,675; and 5,750,341, each of which is incorporated herein by reference in its entirety.

In an aspect is provided a method for making the mixtures of deoxyribonucleotide triphosphates (dNTPs) of one or more of the kits described herein, wherein each mixture of two pluralities of dNTPs is prepared from two individual solutions of dNTPs (e.g., one solution of dATP and one solution of dTTP, or one solution of dCTP and one solution of dGTP), and wherein each mixture of three pluralities of dNTPs is prepared from three individual solutions of dNTPs (e.g., one solution of dATP, one solution of dTTP, and one solution of dGTP; or one solution of dTTP, one solution of dGTP, and one solution of dCTP; or one solution of dATP, one soluditon of dCTP, and one solution of dGTP; or one solution of dTTP, one solution of dCTP, and one solution of dATP). In embodiments, the individual solutions comprising each dNTP (e.g., dATP, dGTP, dCTP, and dTTP) are located on the sequencing device. In embodiments, prior to each sequencing and/or extension cycle, the individual solutions located on the sequencing device are mixed together to form a mixture of two pluralities of dNTPs or a mixture of three pluralities of dNTPs. In embodiments, the individual solutions comprising each dNTP (e.g., dATP, dGTP, dCTP, and dTTP) are located outside of the sequencing device.

### IV. Systems & Devices

The present disclosure also provides systems and devices for implementing methods described herein. In an aspect is a device for sequencing a nucleic acid template, including: i) a reaction vessel for receiving flows of different sequencing solutions, wherein each of a plurality of the sequencing solutions includes a different combination of two to three nucleotide types, wherein each nucleotide of each nucleotide type includes a reversible terminator; ii) a plurality of reservoirs that each contain a different nucleotide type; iii) flow paths from each reservoir to the reaction vessel; and iv) a fluidics controller that controls the flow from the reservoirs to the reaction vessel, wherein the fluidics controller is programmed to randomly provide flow from the plurality of reservoirs to the reaction vessel in order to form the sequencing solutions.

In an aspect is a device for sequencing a nucleic acid template, including: i) a reaction vessel for receiving flows of different sequencing solutions, wherein each of a plurality of the sequencing solutions includes a different combination of two to three nucleotide types, wherein each nucleotide of each nucleotide type includes a reversible terminator; ii) a first reservoir that contains a first sequencing solution and a second reservoir that contains a second sequencing solution, wherein the first and second sequencing solutions collectively include all four nucleotide types; iii) flow paths from each reservoir to the reaction vessel; and iv) a fluidics controller that controls the flow from the reservoirs to the reaction vessel, wherein the fluidics controller is programmed to randomly provide flow from each of the reservoirs to the reaction vessel.

In an aspect is a device for sequencing a nucleic acid template, including: i) a reaction vessel for receiving flows of different sequencing solutions, wherein each of a plurality of the sequencing solutions includes a different combination of four nucleotide types, wherein at least one nucleotide type is a non-incorporating nucleotide type, and the remaining nucleotide types each include a reversible terminator; ii) a first reservoir that contains a first sequencing solution and a second reservoir that contains a second sequencing solution, wherein the first and second sequencing solutions collectively include all four nucleotide types; iii) flow paths from each reservoir to the reaction vessel; and iv) a fluidics controller that controls the flow from the reservoirs to the reaction vessel, wherein the fluidics controller is programmed to randomly provide flow from each of the reservoirs to the reaction vessel.

In an aspect is provided a device for sequencing a nucleic acid template, including: i) a reaction vessel for receiving flows of different solutions, wherein each of a plurality of the solutions includes a different combination of two to three nucleotide types, wherein each nucleotide of each nucleotide type includes a reversible terminator; ii) a plurality of reservoirs that each contain a different nucleotide type; iii) flow paths from each reservoir to the reaction vessel; and iv) a fluidics controller that controls the flow from the reservoirs to the reaction vessel, wherein the fluidics controller is programmed to randomly provide flow from the plurality of reservoirs to the reaction vessel in order to form the solutions.

In an aspect is provided a device for sequencing a nucleic acid template, including: i) a reaction vessel for receiving flows of different solutions, wherein each of a plurality of the solutions includes a different combination of two to three nucleotide types, wherein each nucleotide of each nucleotide type includes a reversible terminator; ii) a first reservoir that contains a first solution and a second reservoir that contains a second solution, wherein the first and second solutions collectively include all four nucleotide types; iii) flow paths from each reservoir to the reaction vessel; and iv) a fluidics controller that controls the flow from the reservoirs to the reaction vessel, wherein the fluidics controller is programmed to randomly provide flow from each of the reservoirs to the reaction vessel.

In an aspect is provided a device for sequencing a nucleic acid template, including: i) a reaction vessel for receiving flows of different solutions, wherein each of a plurality of the solutions includes a different combination of four nucleotide types, wherein at least one nucleotide type is a non-incorporating nucleotide type, and the remaining nucleotide types each include a reversible terminator; ii) a first reservoir that contains a first solution and a second reservoir that contains a second solution, wherein the first and second solutions collectively include all four nucleotide types; iii) flow paths from each reservoir to the reaction vessel; and iv) a fluidics controller that controls the flow from the reservoirs to the reaction vessel, wherein the fluidics controller is programmed to randomly provide flow from each of the reservoirs to the reaction vessel.

As described herein, a solution of nucleotide types may include a sequencing solution (i.e., a solution of nucleotide types used during a cycle that includes detecting a characteristic signature indicating that one to three nucleotides have been incorporated into a primer), or a solution of nucleotide types may include an extension solution (e.g., a solution of nucleotide types lacking a detectable label, and used during a cycle that does not include detecting a characteristic signature).

In an aspect is a system, including: at least one data processor; and at least one memory storing instructions, which when executed by the at least one data processor, result in operations including: obtaining a plurality of sequencing reads from a genomic sequencing device, wherein each sequencing read is generated by executing a plurality of sequencing cycles, each cycle including contacting the nucleic acid template with a sequencing solution in the presence of a polymerase, wherein each sequencing solution includes four nucleotide types; and detecting a characteristic signature indicating that the nucleotide has been incorporated into the sequencing primer; and obtaining a plurality of sequencing reads from a genomic sequencing device, wherein each sequencing read is generated according to any one of the methods of the invention, and embodiments described herein.

In an aspect is a system, including: at least one data processor; and at least one memory storing instructions, which when executed by the at least one data processor, result in operations including: obtaining a plurality of sequencing reads from a genomic sequencing device, wherein each sequencing read is generated by executing a plurality of sequencing cycles, each cycle including contacting the nucleic acid template with a sequencing solution in the presence of a polymerase, wherein each sequencing solution includes four nucleotide types, wherein at least one nucleotide type is a non-incorporating nucleotide type, and the remaining nucleotide types each include a reversible terminator; and detecting a characteristic signature indicating that the nucleotide has been incorporated into the sequencing primer; and obtaining a plurality of sequencing reads from a genomic sequencing device, wherein each sequencing read is generated according to any one of the methods of the invention, and embodiments described herein.

One or more aspects or features of the subject matter described herein may be realized in digital electronic circuitry, integrated circuitry, specially designed ASICs (application specific integrated circuits), computer hardware, firmware, software, and/or combinations thereof. These various implementations may include implementation in one or more computer programs that are executable and/or interpretable on a programmable system including at least one programmable processor, which may be special or general purpose, coupled to receive data and instructions from, and to transmit data and instructions to, a storage system, at least one input device (e.g., mouse, touch screen, etc.), and at least one output device. The methods and systems described herein can be implemented or performed by a machine, such as a processor configured with specific instructions, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor. A processor can also be implemented as a combination of computing devices, e.g., a combination of a DSP and a microprocessor, a plurality of microprocessors, one or more microprocessors in conjunction with a DSP core, or any other such configuration. The elements of a method or process as described herein can be implemented within computational hardware, in a software module executed by a processor, or in a combination of the two. A software module can reside in RAM memory, flash memory, ROM memory, EPROM memory, EEPROM memory, registers, hard disk, a removable disk, a CD-ROM, or any other form of computer-readable storage medium known in the art.

The computer programs, which can also be referred to as programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid state memory or a magnetic hard drive or any equivalent storage medium. The machine-readable medium can alternatively or additionally store such machine instructions in a transient manner, such as for example as would a processor cache or other random access memory associated with one or more physical processor cores. The computer can run any one of a variety of operating systems, such as for example, any one of several versions of Windows, or of MacOS, or of Unix, or of Linux.

With certain aspects, to provide for interaction with a user, the subject matter described herein can be implemented on a computer having a display device, such as for example a cathode ray tube (CRT) or a liquid crystal display (LCD) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including, but not limited to, acoustic, speech, or tactile input. Other possible input devices include, but are not limited to, touch screens or other touch-sensitive devices such as single or multi-point resistive or capacitive trackpads, voice recognition hardware and software, optical scanners, optical pointers, digital image capture devices and associated interpretation software, and the like.

The subject matter described herein may be implemented in a computing system that includes a back-end component (e.g., as a data server), or that includes a middleware component (e.g., an application server), or that includes a front-end component (e.g., a client computer having a graphical user interface or a Web browser through which a user may interact with an implementation of the subject matter described herein), or any combination of such back-end, middleware, or front-end components. The components of the system may be interconnected by any form or medium of digital data communication (e.g., a communication network). Examples of communication networks include a local area network (“LAN”), a wide area network (“WAN”), the Internet, WiFi (IEEE 802.11 standards), NFC, BLUETOOTH, ZIGBEE, and the like.

The computing system may include clients and servers. A client and server are generally remote from each other and typically interact through a communication network. The relationship of client and server arises by virtue of computer programs running on the respective computers and having a client-server relationship to each other.

### EXAMPLES

### Example 1. Modeling Dephasing During Sequencing-y-Synthesis

Traditional sequencing-by-synthesis (SBS) methodologies employ serial incorporation and detection of labeled nucleotide analogues into a primer hybridized to a target nucleic acid sequence. For example, high-throughput SBS technology uses cleavable fluorescent nucleotide reversible terminator (NRT) sequencing chemistry. These cleavable fluorescent NRTs were designed based on the following rationale: each of the four nucleotides (deoxyadenosine (dA), deoxycytidine (dC), deoxyguanosine (dG), deoxythymidine (dT), and/or deoxyuridine (dU)) is modified by attaching a unique cleavable fluorophore to the specific location of the nucleobase and capping the 3′—OH group of the nucleotide sugar with a small reversible moiety (also referred to herein as a reversible terminator) so that they are still recognized by DNA polymerase as substrates. The reversible terminator temporarily halts the polymerase reaction after nucleotide incorporation while the fluorophore signal is detected. After incorporation and signal detection, the fluorophore and the reversible terminator are cleaved to resume the polymerase reaction in the next cycle.

In some embodiments, the NRT of the present invention includes a 3′-unblocked nucleotide and a linker-based reversible terminator, for example, the “virtual terminator” as described in U.S. Pat. No. 8,114,973 and the “lightening terminator” as described in U.S. Pat. No. 10,041,115, the contents of which are incorporated herein by reference in their entirety. In this case, the reversible termination group is the dye-linker combination attached to the nucleobase which functions to temporarily terminate the primer extension. Nucleotides containing a 3′-unblocked label-based reversible terminator does not require a mutant DNA polymerase to incorporate the nucleotide into the primer due to the lack of a modified moiety at the 3′—OH (see, Chen F et al. Genomics Proteomics Bioinformatics, 2013, 11(1)34-40, or Bowers J et al. Nat. Methods, 2009, 6(8): 593-595). Both the 3′—O—reversible terminator and the linker-based reversible terminator nucleotides temporarily halts the polymerase reaction after nucleotide incorporation while the fluorophore signal is detected. After incorporation and signal detection, the terminating and fluorophore groups are cleaved to resume the polymerase reaction in the next cycle.

Typically, many polynucleotides are confined to an area of a discrete region, referred to as a cluster, and are synchronized in their nucleotide incorporation and detection. For example, at the start of a sequencing reaction, after hybridization of the sequencing primer, 100% of the strands within the cluster are synchronized. As the strands are extended, individual strands may fall behind or extend faster than the majority of the strands in the cluster, referred to as dephasing. This loss of synchronization is amplified as the number of sequencing rounds increases and eventually, the background noise from the unsynchronized strands becomes too great to accurately call the correct base. Some strands may extend faster when the reversible terminator of the nucleotide to be incorporated is removed prematurely, or the solution of reversibly terminated nucleotides contains impurities (e.g., natural nucleotides or modified nucleotides bearing a 3′ hydroxyl group), resulting in the clusters of monoclonal amplicons being out-of-phase.

During SBS, dephasing leads to signal loss and lowered base call accuracy that restricts the maximum read length produced by a sequencing device. Typically, a sequencing cycle introduces all four labeled nucleotide types (e.g., dA, dT, dC, and dG) simultaneously into a flow cell. Such an approach leaves no opportunity for out of phase sequences to resynchronize. To increase sequencing efficiency, accuracy, and permit longer sequencing reads, there is a need for new methods to correct dephasing. For example, within typical sequencing-by-synthesis modalities, a large population of substantially identical template polynucleotide strands (e.g., 10³to 10⁷molecules) are analyzed substantially simultaneously in a given sequencing reaction to obtain sufficiently distinct and resolvable signals for reliable detection. Ensemble-based SBS includes sequencing collections of identical sequences (i.e., monoclonal clusters of amplicons) and determining their sequence by synthesis of the complement in a stepwise, synchronous fashion. This results in an average sequence signal from all the amplicons present in a cluster per incorporation event. Signal-to-noise ratios may be improved when there is homogeneous and/or contemporaneous extension of the complementary strand associated with the template molecules in a population. Each extension reaction associated with the population of template molecules may be described as being generally “in-phase” or having “phasic synchrony” with each other when they are performing the same incorporation step at the same sequence position for the associated template molecules in a given reaction step. It has been observed, however, that a relatively small fraction of template molecules in each population may lose or fall out of phasic synchrony (e.g., may become “out of phase”) with the majority of the template molecules in the population. That is, the incorporation events associated with a certain fraction of template molecules may either get ahead of or fall behind other similar template molecules in the sequencing run. Such phase loss effects limits the sequencing read lengths in commercial sequencing platforms and are described in Ronaghi, GENOME RESEARCH, 11:3-11 (2001); Leamon et al., CHEMICAL REVIEWS, 107:3367-3376 (2007); and Chen et al., International Publ. No. WO 2007/098049; each of which are incorporated herein by reference in their entirety.

One such phase loss effect relates to an “incomplete extension” (IE) event or error (also referred to herein as a “lag error”). An IE event may occur as a result of a failure of a sequencing reaction to incorporate one or more nucleotide species into one or more nascent molecules for a given extension round of the sequence, for example, which may result in subsequent reactions being at a sequence position that is out of phase with the sequence position for the majority of the population (e.g., certain template extensions fall behind the main template population). IE events may arise, for example, due of a lack of nucleotide availability to a portion of the template/polymerase complexes of a population. Alternatively, or in addition, IE events may be caused by a defective or absent polymerase, or an incorporated nucleotide that does not have a 3′ OH available (e.g., retains a reversible terminator) for nucleotide polymerization.

Another such phase loss effect relates to a “carry forward” (CF) event or error (also referred to herein as a “lead error”). A CF event may occur as a result of an improper additional extension of a nascent molecule by incorporation of one or more nucleotide species in a sequence or strand position that is ahead and thus out of phase with the sequence or strand position of the rest of the population. CF events may arise, for example, because of the misincorporation of a nucleotide species, or in certain instances, because of contamination from nucleotides remaining from a previous cycle (e.g., which may result from an insufficient or incomplete washing of the reaction chamber). For example, a small fraction of a “dT” nucleotide cycle may be present or carry forward to a “dC” nucleotide cycle. The presence of both nucleotides may lead to an undesirable extension of a fraction of the growing strands where the “dT” nucleotide is incorporated in addition to the “dC” nucleotide such that multiple different nucleotide incorporations events take place where only a single type of nucleotide incorporation would normally be expected. Alternatively, some strands may extend faster when the reversible terminator of the nucleotide to be incorporated is removed prematurely, or the solution of reversibly terminated nucleotides contains impurities (e.g., natural nucleotides or modified nucleotides bearing a 3′ hydroxyl group). CF events may also arise because of a polymerase error (e.g., there may be an improper incorporation of a nucleotide species into the nascent molecule that is not complementary to the nucleotide species on the template molecule).

Errors or phasing issues related to IE and CF events may be exacerbated over time because of the accumulation of such events, which may cause degradation of sequence signal or quality over time and an overall reduction in the practical read length of the system (e.g., the number of nucleotides that can be sequenced for a given template). The present disclosure reflects the discovery that sequencing performance (e.g., efficiency and/or accuracy of sequencing) may be affected by the particular composition, nature, and flow sequence of nucleotides delivered to sequencing-by-synthesis reactions.

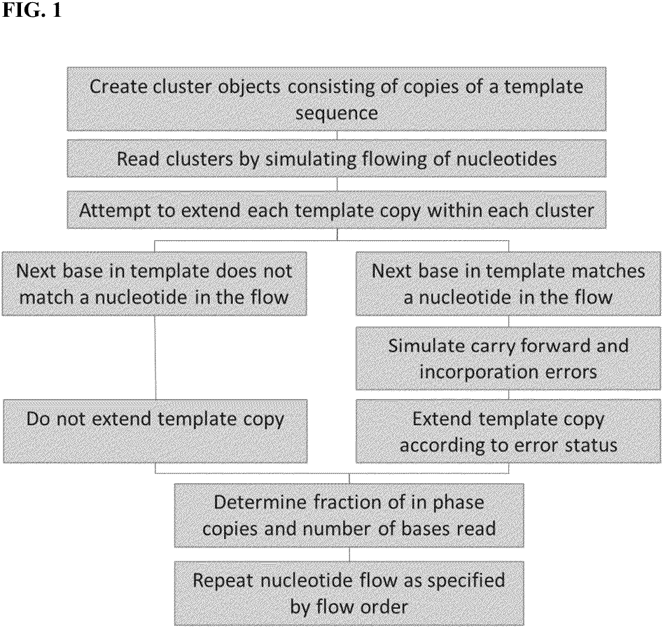

To better understand the impact of nucleotide flow orders on dephasing, we created a simulation to model dephasing during sequencing-by-synthesis using different reagent flows. As described elsewhere herein, we modeled conventional predetermined reagent flow orders (e.g., de Bruijn and Samba flow order schemes), random flow orders, and default reagent flow (i.e., all four nucleotide types simultaneously). An overview of the process is presented in FIG. 1. The simulation consists of 1,000 cluster objects each composed of 1,000 copies of a 1,000 base template sequence of the four DNA bases in a random sequence (i.e., non-genomic sequence). During the simulation, the cluster template sequences are read by successively exposing the clusters to up to four nucleotides as indicated by a given nucleotide flow order. For each of the 1,000 template copies within a cluster, the simulation asks whether the next nucleotide matches one of the nucleotides present in the cycle and whether a lead error (incorporation of two bases) or lag error (failure to incorporate a base) has occurred, modeling each as a random process with 1% error probability. If an error does not occur, a single base is added to the extending template copy. After each successive cycle, the simulation determines the average fraction of template copies lacking lead or lag errors (in-phase templates), as well as the average number of bases incorporated (i.e., read) across all clusters.

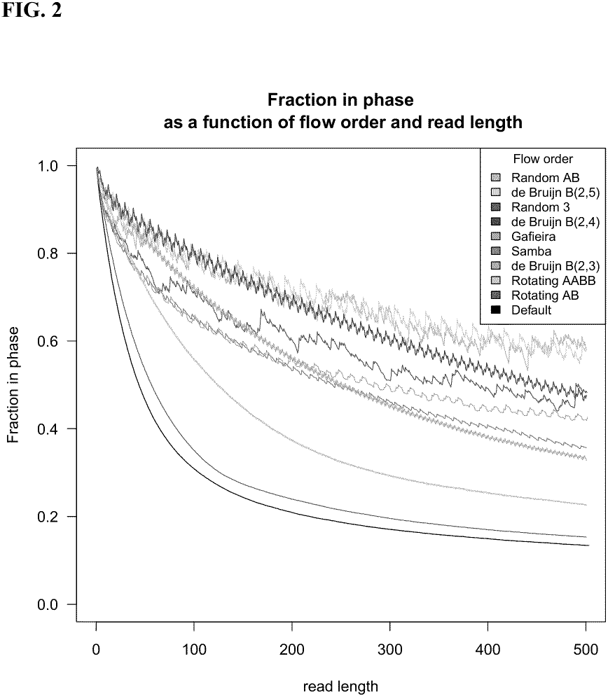

The performance of a variety of reagent flow orders over the course of a 500 base incorporation simulated sequencing experiment is shown in FIG. 2. The predetermined reagent flow orders include de Bruijn flow orders, (e.g., de Bruijn B(2,5), de Bruijn B(2,4), and de Bruijn(2,3) flow orders), Samba, and Gafieira. As described in Bragg et al. (Bragg, L. M., Stone, G., Butler, M. K., Hugenholtz, P., & Tyson, G. W. (2013). 9(4), e1003031) and U.S. Patent Application Publication US2012/0264621, Samba is a complex flow cycle having a period of 32, Gafieira having a period of 48, with a pattern that repeats some nucleotides in a period shorter than four. For example, an exemplary Samba flow ordering may include flowing nucleotides in the order “TACG, TACG, TCTG, AGCA, TCGA, TCGA, TGTA, CAGC”, and an exemplary Gafieira flow ordering may include flowing one nucleotide at a time in the order “TACG, TACG, TCTG, AGCA, TCGA, TCGA, TGTA, CAGC, TGAC, TGAC, TATC, GCAG, AGCT, AGCT, ACAT, GTCG, ACTG, ACTG, ATAG, CGTC, ATGC, ATGC, AGAC, TCGT, CGTA, CGTA, CTCA, GATG, CTAG, CTAG, CACG, TGAT, CAGT, CAGT, CGCT, ATGA, GTCA, GTCA, GCGA, TACT, GCAT, GCAT, GAGT, CTAC, GATC, GATC, GTGC, ACTA.” The de Bruijn sequences have the nomenclature B(k,n), where k denotes a size of an alphabet (e.g., an alphabet of all possible nucleotides, dA, dT, dC, dG, and dU) and n denotes a length of subsequences in the alphabet. The sequences B(k,n) are such that every possible subsequence of length n in the alphabet appears exactly once as a sequence of consecutive characters. For example, de Bruijn B(2,5), de Bruijn B(2,4), and de Bruijn B(2,3) flow order implies two distinct reagent solutions, i.e., reagent A and reagent B, flowed in different combinations. The de Bruijn B(2,3) flow order cycles through “A, A, A, B, A, B, B, B” where A and B represent reagent A and reagent B, each reagent containing two nucleotides. Similarly, the de Bruijn B(2,4) flow order cycles through “A, A, A, A, B, B, B, B, A, B, B, A, A, B, A, B” where A and B represent reagent A and reagent B, each reagent containing two nucleotides. Note, the identity of the two nucleotides remains constant throughout the period of cycles. More information about de Bruijn sequences and related concepts may be found in Ehrenfest and de Bruijn, Circuits and Trees in Oriented Linear Graphs, Simon Stevin, 28:203-217 (1951); and de Bruijn, Acknowledgement of Priority to C. Flye Sainte-Marie on the Counting of Circular Arrangements of 2ⁿZeros and Ones that Show Each N-Letter Word Exactly Once, T.H.-Report 75-WSK-06, Technological University Eindhoven (1975), which are both incorporated by reference herein in their entirety. Further, Random AB refers to a random selection between two reagents, reagent A which contains two nucleotides types (e.g., dA and dC); and reagent B includes two other nucleotides (e.g., dT and dG). Alternating between reagent A and reagent B in a repeated ordering of AABB is referred to as Rotating AABB; similarly, Rotating AB refers to a regular alternation between two reagents, reagent A and reagent B. Note, reagent A and reagent B each consist of two of the four nucleotides and remain identical over the course of the cycles. Random 3 refers to a random selection of three of the four nucleotides, wherein all three nucleotides are simultaneously introduced each flow (e.g., flow 1 is dA, dT, and dC; flow 2 is dA, dC, and dG, etc.).

The practicality of the described alternative nucleotide flow orders is informed in part by the mechanical complexity required to implement the nucleotide flow on a sequencing instrument. For example, a default four nucleotide flow order provides no protection against dephasing, but can be implemented by exposing a flow cell to a single nucleotide reagent solution consisting of all four nucleotides. By contrast, implementing the Random 3 flow order would require either exposing the flow cell to a random selection of one of four solutions, each containing a different three nucleotide combination, or alternatively, random selection of three of four single nucleotide solutions, which are then combined as they are introduced onto the flow cell. Thus, the Random 3 flow order, and other flow orders employing three nucleotides per flow, require sequencing machine fluidics to accommodate regulation of a minimum of four separate nucleotide reagent solutions. A mechanically simpler solution is to alternate between two nucleotide reagent solutions, each containing a subset of the four nucleotide bases (for example dA/dG in solution ‘A’ and dT/dC in solution ‘B’). This implementation requires fluidics to support the regulation of two as opposed to four nucleotide reagent solutions. Further, for sequencing systems employing fluorophore-labeled nucleotides detected following laser excitation, a system employing two two-nucleotide reagent solutions requires laser excitation of only two fluorophores per flow cycle. This can be accomplished by excitation via a single laser, rather than the two lasers typically required to detect four fluorophores in a traditional four nucleotide flow paradigm. Two-color sequencers have only two channels and therefore take only two images of the same portion of the flow cell. For example, a two-channel sequencer uses a mix of dyes for each base and uses red and green filters for the two images. In embodiments using a two-channel sequencer, clusters seen in red or green images are interpreted as dC and dT bases, respectively when flowing a first sequencing solution containing dC labeled with a red detectable moiety and dT labeled with a green detectable label. Following incubation with a second sequencing solution, where dA is labeled with a red detectable moiety and dG is labeled with a green detectable label, clusters observed in in both red and green images are flagged as dA and dG bases.

In light of the increased mechanical complexity required to implement alternative nucleotide flows, it is preferred to identify flow orders that maximize desirable sequencing characteristics (e.g., accuracy, speed, read length) while minimizing the requisite instrument complexity. Importantly, the results of this work suggest that non-predetermined flow orders, such as those defined by a random alternation between two nucleotide reagent solutions, afford superior phase protection compared to predetermined orders, while maintaining a relatively high sequencing efficiency and minimizing complexity of the sequencing device.

The phase protective capacity of a two-solution implementation may be further improved upon by selecting nucleotide pairings that employ a two-reagent solution, wherein one solution contains purine nucleotides (i.e., adenine and guanine) and a second solution contains pyrimidine nucleotides (i.e., cytosine and thymine). Polymerases typically make transition errors (purine for purine) more than transversion errors (purine for pyrimidine). A benefit of having two structurally similar nucleotide solutions (e.g., a first solution includes adenine and guanine, or analogues thereof, and a second solution contains cytosine and thymine, or analogues thereof) is the polymerase may substitute a structurally similar nucleotide into the extending primer and maintain synchrony among the clusters. Any infrequent transition errors can subsequently be corrected bioinformatically. Alternatively, in embodiments, one solution contains structurally dissimilar nucleotides (e.g., adenine and thymine) and a second solution contains structurally dissimilar nucleotides (e.g., cytosine and guanine). Two structurally dissimilar solutions minimize transition errors.

An alternate two-solution implementation that may further improve upon the phase protective capacity of the two-reagent solution described supra employs non-incorporating nucleotides (e.g., non-hydrolyzable). For example, one solution contains purine nucleotides (e.g., adenine and guanine), each including a reversible terminator, and non-incorporating pyrimidine nucleotides (e.g., non-hydrolyzable cytosine and thymine). A benefit of having the non-incorporating nucleotides present is that they serve as competitors to reduce polymerase misincorporation errors. Alternatively, in embodiments, one solution contains structurally dissimilar nucleotides (e.g., adenine and thymine), each including a reversible terminator, and structurally dissimilar non-incorporating nucleotides (e.g., non-hydrolyzable cytosine and guanine).

Table 1 summarizes the simulation results by reporting: (1) the number of nucleotides flowed per cycle; (2) the fraction of in phase templates when an average read length of 500bp has been achieved; (3) the number of sequencing cycles required to achieve a read length of 500bp; (4) the increase in sequencing cycles compared to the default four nucleotide flow; and (5) the average fraction of in phase templates across all cycles of the simulation, reflective of the area under the curve for each flow order. The results from these simulations suggest that nucleotide flow orders derived from random selection of nucleotides (e.g., Random AB or Random 3) have phase protection superior to the standard four nucleotide flow. Further, we observed that as read length increases, random flow orderings appear to provide superior phase protection compared to many other orders, while also maintaining a relatively high sequencing efficiency. Flow orderings derived from higher order de Bruijn sequences (orders 6, 7, 8, 9, 10, 11) did not show improved performance compared to the Random AB and de Bruijn B(2,5) flow orders at a read length of 500 bases (data not shown). As discussed infra, Thue-Morse sequences may be particularly advantageous in the context of a two-solution sequencing paradigm, with respect to phase protection, sequencing efficiency, and compatibility with reference-free error correction strategies. The simulation assumes a lead and lag error rate of 1% each per base per flow. Results are derived from one simulation for each flow type, with the exception of the Random AB flow order, where results from two simulations are provided.

TABLE 1

Phase protection and sequencing efficiency for selected flow orders

| Flow Name | Nucleotides per flow | Fraction in phase at average read length of 500bp | Number of cycles to achieve 500bp average read length | Increase in sequencing time compared to default flow | Avg fraction in phase templates per cycle at avg read length of 500bp |
|---|---|---|---|---|---|
| Default | 4 | 13.3% | 500 | 0% | 0.25 |
| Thue-Morse 4 | 2 | 79.2% | 1602 | 220% | 0.869 |
| Thue-Morse 3 | 2 | 72.3% | 1296 | 160% | 0.813 |
| Thue-Morse 2 | 2 | 63.0% | 1026 | 105% | 0.74 |
| Random AB sim1 | 2 | 58.0% | 987 | 97% | 0.705 |
| Random AB sim2 | 2 | 60.0% | 997 | 99% | 0.691 |
| de Bruijn B(2,5) | 2 | 57.7% | 1005 | 101% | 0.706 |
| Random 3 | 3 | 48.6% | 675 | 35% | 0.6 |
| de Bruijn B(2,4) | 2 | 46.7% | 986 | 97% | 0.655 |
| Thue-Morse | 2 | 42.9% | 819 | 64% | 0.576 |
| Gafieira | 3 | 42.6% | 657 | 31% | 0.555 |
| Samba | 3 | 35.6% | 639 | 28% | 0.532 |
| de Bruijn B(2,3) | 2 | 32.8% | 935 | 87% | 0.549 |
| Rotating AABB | 2 | 22.5% | 840 | 68% | 0.406 |
| Rotating AB | 2 | 15.3% | 756 | 50% | 0.283 |

### Simulating Performance at Very Long Read Lengths

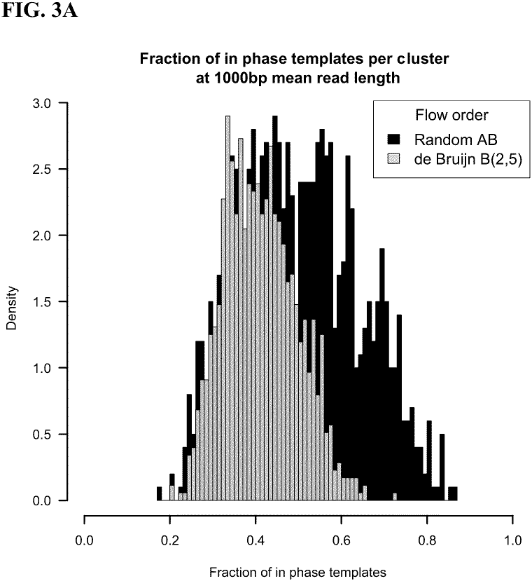

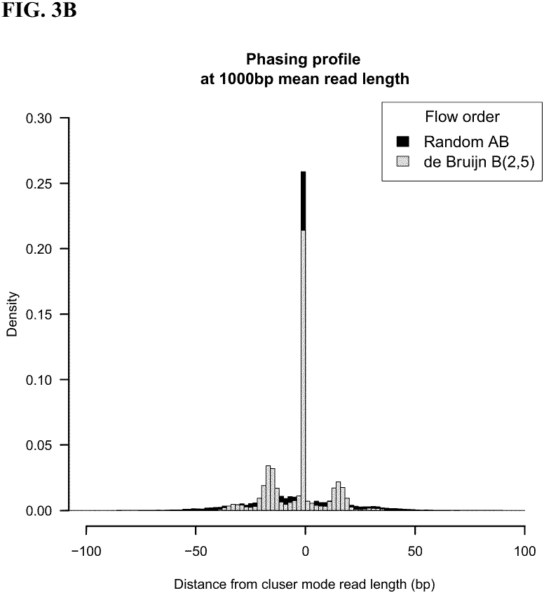

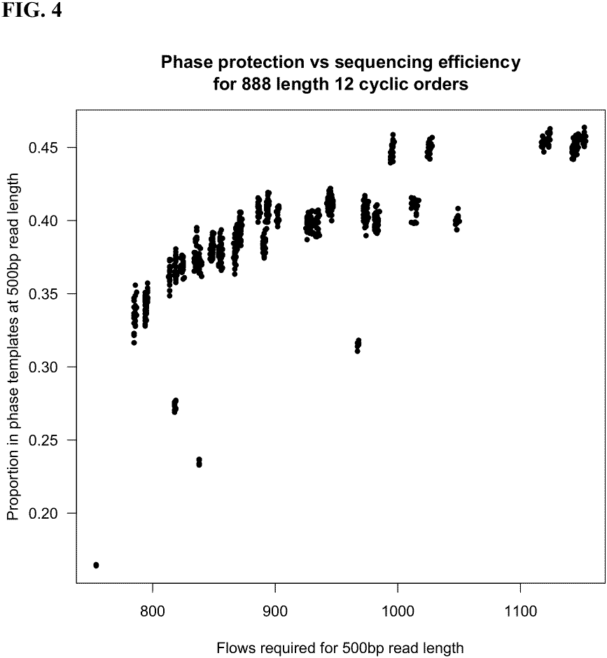

The results presented in Table 1 imply roughly equivalent performance of the Random AB and de Bruijn B(2,5) sequences with respect to phase protection and sequencing efficiency at simulated read lengths of 500bp. To further explore potential differences between these flows, we repeated the simulation, this time extending the simulation until a mean read length of 1000bp was achieved via the Random AB and B(2,5) flow orders. For each flow order, we determined the fraction of in phase templates across the 1000 clusters within the simulation, then compared the resultant distributions (FIG. 3A). At 1000bp, the random flow order showed a higher overall fraction of in phase templates compared to the de Bruijn B(2,5) sequence derived flow order (mean 50.1% vs 41.2%, respectively), but also a higher variance across clusters (standard deviations of .138 and .0849, respectively). Consequently, the random flow order yielded a significantly larger fraction of clusters maintaining a high level of synchronization, here defined as those having > 60% in phase templates (25.4% vs 1.6%, respectively). To the extent that clusters having highly synchronized templates yield a more accurate read sequence, this result suggests that the random flow order may outperform de Bruijn sequences, particularly at longer read lengths.

We next examined the extent of dephasing for each of the 1000 extending template sequences within each cluster in the simulation, quantified as the number of the base pairs a template sequence was ahead or behind of the mode template length for the cluster (FIG. 3B). Surprisingly, we find that the de Bruijn B(2,5) flow order tends to resynchronize out of phase templates such that they are maintained either ahead or behind the main population by a fixed number of bases in a manner proportional to the length of the de Bruijn sequence. By contrast, the Random AB flow order appears to yield a wide distribution of offsets among out of phase templates, and an overall higher fraction of in phase templates. Synchronization of out of phase templates may be problematic for base calling, as it results in a greater variance in channel signal intensity noise, thus increasing the likelihood of misidentifying the signal deriving from the main population of in phase templates. In the same way, such increased variance would have a direct effect on the variance in the ratio of the strongest channel signal to the second strongest channel signal, a metric which has been widely employed as an indicator of naive base quality on the Illumina platform, and other ensemble sequencing platforms (e.g., Omniome, see, for example, U.S. Pat. 10,731,141). In total, these results suggest that random flow orders may be especially well suited to the production of long sequence reads, potentially enabling read lengths that exceed the limit achievable by current ensemble sequencing by synthesis methods.

### Systematic Evaluation of Two Solution Flow Orders

The results described herein, for example FIG. 3B, suggests that random flow orders of sequencing solutions may be superior to de Bruijn sequence-derived flow orders with respect to phase protection and sequencing efficiency within a two-solution (i.e., binary) system. Given that de Bruijn sequences only a small fraction of available flow orders, we next systematically examined the simulated performance of cyclic repetitions of every potential permutation of a 12-flow order consisting of 6 flows each of solutions A and B. For each of the 888 resultant flow orders, performance was compared based on (1) the number of flows required to achieve a mean read length of 500bp, (2) the fraction of in phase templates at a read length of 500bp, and (3) the proportion of clusters having >60% templates in phase at a read length of 500bp. For each flow, we also noted the number of consecutive flows of the same solution over a 50-cycle flow of each flow order. We found that the number of flows required to achieve a mean read length of 500bp was highly correlated with the fraction of in phase templates at a read length of 500bp (r = 0.78) and the number of consecutive flows of the same solution (r = 0.79). The proportion of clusters having >60% templates in phase was also correlated with the number of consecutive flows of the same solution, albeit to a lower extent (r = 0.32). These results indicate that sequences consisting of consecutive flows of the same base tend to provide greater phase protection than orders consisting of regular alternations between the two solutions, generally at the expense of sequencing efficiency.

### Thue-Morse Sequences Are Phase Protective and Self-Correcting

Given the advantages of consecutive flows of the same nucleobase, the undesirable tendency of cyclic flow orders to synchronize out of phase templates, and the need to produce a flow order that does not bias towards or against specific sequence motifs, we reasoned that Thue-Morse infinite binary sequences could yield advantageous flow orders. Thue-Morse sequences are enriched for consecutive repetitions of the same item and have been shown to yield a balanced alternation between two items irrespective of sequence length (Richman, R. “Recursive Binary Sequences of Differences” (2001). Complex Systems. 13 (4):381-382 ). We created Thue-Morse sequences using: (a) one cycle of a single solution (‘A’) as the starting unit (see, Thue-Morse, Table 2), such that the first 8 cycles consist of: A, B, B, A, B, A, A, B, where ‘A’ and ‘B’ refer to two different sequencing solutions, e.g. ‘dA, dT’ and ‘dC, dG’; (b) two cycles of a single solution (‘A’,‘A’) as the starting unit (see, Thue-Morse 2, Table 2), such that the first 8 cycles consist of: A, A, B, B, B, B, A, A, where ‘A’ and ‘B’ refer to two different sequencing solutions; (c) three cycles of a single solution (‘A’,’A’,’A’) as the starting unit (see, Thue-Morse 3, Table 2), such that the first 8 cycles consist of: A, A, A, B, B, B, B, A, where ‘A’ and ‘B’ refer to two different sequencing solutions; (d) four cycles of a single solution (‘A’,’A’,’A’,’A’) as the starting unit (see, Thue-Morse 4, Table 2), such that the first 8 cycles consist of: A, A, A, A, B, B, B, B, where ‘A’ and ‘B’ refer to two different sequencing solutions; and (e) by transforming the Thue-Morse 2 sequence above such that each instance of four repeats of the same solution is reduced to three repeats of that solution (see, Thue-Morse 2b, Table 2, first 8 cycles of: A, A, B, B, B, A, A, B ).

TABLE 2

Thue-Morse sequences. Each doublet (i.e., two nucleobases) is separated by a comma to indicate separate solutions. For example, the first sequence Thue-Morse employs a first extension solution including dATP and dTTP followed by a second extension solution including dCTP and dGTP

| | |
|---|---|
| Sequence Name | Flow order sequence (First 3000bp) |
| Thue-Morse | AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, CG, AT, AT, CG, AT, CG, CG, AT, AT, CG, CG, AT, CG, AT, AT, CG, AT, CG, CG, AT, CG, AT, AT, CG, CG, AT, AT, CG, AT, CG, CG, AT, AT, CG, CG, AT, CG, AT, AT, CG, CG, AT, AT, CG, AT, CG, CG, AT, CG, AT, AT, CG, AT, CG, CG, AT, AT, CG, CG, AT, CG, AT, AT, CG, CG, AT, AT, CG, AT, CG, CG, AT, AT, CG, CG, AT, CG, AT, AT, CG, AT, CG, CG, AT, CG, AT, AT, CG |
| Thue-Morse2 | AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, CG, CG, AT, AT, AT, AT, CG, CG, AT, AT, CG, CG, CG, CG, AT, AT, AT, AT, CG, CG, CG, CG, AT, AT, CG, CG, AT, AT, AT, AT, CG, CG, AT, AT, CG, CG, CG, CG, AT, AT, CG, CG, AT, AT, AT, AT, CG, CG, CG, CG, AT, AT, AT, AT, CG, CG, AT, AT, CG, CG, CG, CG, AT, AT, CG, CG, AT, AT, AT, AT, CG, CG, AT, AT, CG, CG, CG, CG, AT, AT, AT, AT, CG, CG, CG, CG, AT, AT |
| Thue-Morse 2b | AT, AT, CG, CG, CG, AT, AT, CG, CG, AT, AT, AT, CG, CG, CG, AT, AT, AT, CG, CG, AT, AT, CG, CG, CG, AT, AT, CG, CG, AT, AT, AT, CG, CG, AT, AT, CG, CG, CG, AT, AT, AT, CG, CG, CG, AT, AT, CG, CG, AT, AT, AT, CG, CG, CG, AT, AT, AT, CG, CG, AT, AT, CG, CG, CG, AT, AT, AT, CG, CG, CG, AT, AT, CG, CG, AT, AT, AT, CG, CG, AT, AT, CG, CG, CG, AT, AT, CG, CG, AT, AT, AT, CG, CG, CG, AT, AT, AT, CG, CG, AT, AT, CG, CG, CG, AT, AT, CG, CG, AT, AT, AT, CG, CG, AT, AT, CG, CG, CG, AT, AT, AT, CG, CG, CG, AT, AT, CG, CG, AT, AT, AT, CG, CG, AT, AT, CG, CG, CG, AT, AT, CG, CG, AT, AT, AT, CG, CG, CG, AT, AT, AT, CG, CG, AT, AT, CG, CG, CG, AT, AT, AT, CG, CG, CG, AT, AT, CG, CG, AT, AT, AT, CG, CG, CG, AT, AT, AT, CG, CG, AT, AT, CG, CG, CG, AT, AT, CG, CG, AT, AT, AT, CG, CG, AT, AT, CG, CG, CG, AT, AT, AT, CG, CG, CG, AT, AT, CG, CG, AT, AT, AT, CG, CG, CG, AT, AT, AT, CG, CG, AT, AT, CG, CG, CG, AT, AT, AT, CG, CG, CG, AT, AT, CG, CG, AT, AT, AT, CG, CG, AT, AT, CG, CG, CG, AT, AT, CG, CG, AT, AT, AT, CG, CG, CG, AT, AT, AT, CG, CG, AT, AT, CG, CG, CG, AT, AT, AT, CG, CG, CG, AT, AT, CG, CG, AT, AT, AT, CG, CG, CG, AT, AT, AT, CG, CG, AT, AT, CG, CG, CG, AT, AT, CG, CG, AT, AT, AT, CG, CG, AT, AT, CG, CG, CG, AT, AT, AT, CG, CG, CG, AT, AT, CG, CG, AT, AT, AT, CG, CG, AT, AT, CG, CG, CG, AT, AT, CG, CG, AT, AT, AT, CG, CG, CG, AT, AT, AT, CG, CG, AT, AT, CG, CG, CG, AT, AT, CG, CG, AT, AT, AT, CG, CG, AT, AT, CG, CG, CG, AT, AT, AT, CG, CG, CG, AT, AT, CG, CG, AT, AT, AT, CG, CG, CG, AT, AT, AT, CG, CG, AT, AT, CG, CG, CG, AT, AT, AT, CG, CG, CG, AT, AT, CG, CG, AT, AT, AT, CG, CG, AT, AT, CG, CG, CG, AT, AT, CG, CG, AT, AT, AT, CG, CG, CG, AT, AT, AT, CG, CG, AT, AT, CG, CG, CG, AT, AT, CG, CG, AT, AT, AT, CG, CG, AT, AT, CG, CG, CG, AT, AT, AT, CG, CG, CG, AT, AT, CG, CG, AT, AT, AT, CG, CG, AT, AT, CG, CG, CG, AT, AT, CG, CG, AT, AT, AT, CG, CG, CG, AT, AT, AT, CG, CG, AT, AT, CG, CG, CG, AT, AT, AT, CG, CG, CG, AT, AT, CG, CG, AT, AT, AT, CG, CG, CG, AT, AT, AT, CG, CG, AT, AT, CG, CG, CG, AT, AT, CG, CG, AT, AT, AT, CG, CG, AT, AT, CG, CG, CG, AT, AT, AT, CG, CG, CG, AT, AT, CG, CG, AT, AT, AT, CG, CG, AT, AT, CG, CG, CG, AT, AT, CG, CG, AT, AT, AT, CG, CG, CG, AT, AT, AT, CG, CG, AT, AT, CG, CG, CG, AT, AT, CG, CG, AT, AT, AT, CG, CG, AT, AT, CG, CG, CG, AT, AT, AT, CG, CG, CG, AT, AT, CG, CG, AT, AT, AT, CG, CG, CG, AT, AT, AT, CG, CG, AT, AT, CG, CG, CG, AT, AT, AT, CG, CG, CG, AT, AT, CG, CG, AT, AT, AT, CG, CG, AT, AT, CG, CG, CG, AT, AT, CG, CG, AT, AT, AT, CG, CG, CG, AT, AT, AT, CG, CG, AT, AT, CG, CG, CG, AT, AT, AT, CG, CG, CG, AT, AT, CG, CG, AT, AT, AT, CG, CG, CG, AT, AT, AT, CG, CG, AT, AT, CG, CG, CG, AT, AT, CG, CG, AT, AT, AT, CG, CG, AT, AT, CG, CG, CG, AT, AT, AT, CG, CG, CG, AT, AT, CG, CG, AT, AT, AT, CG, CG, CG, AT, AT, AT, CG, CG, AT, AT, CG, CG, CG, AT, AT, AT, CG, CG, CG, AT, AT, CG, CG, AT, AT, AT, CG, CG, AT, AT, CG, CG, CG, AT, AT, CG, CG, AT, AT, AT, CG, CG, CG, AT, AT, AT, CG, CG, AT, AT, CG, CG, CG, AT, AT, CG, CG, AT, AT, AT, CG, CG, AT, AT, CG, CG, CG, AT, AT, AT, CG, CG, CG, AT, AT, CG, CG, AT, AT, AT, CG, CG, AT, AT, CG, CG, CG, AT, AT, CG, CG, AT, AT, AT, CG, CG, CG, AT, AT, AT, CG, CG, AT, AT, CG, CG, CG, AT, AT, AT, CG, CG, CG, AT, AT, CG, CG, AT, AT, AT, CG, CG, CG, AT, AT, AT, CG, CG, AT, AT, CG, CG, CG, AT, AT, CG, CG, AT, AT, AT, CG, CG, AT, AT, CG, CG, CG, AT, AT, AT, CG, CG, CG, AT, AT, CG, CG, AT, AT, AT, CG, CG, CG, AT, AT, AT, CG, CG, AT, AT, CG, CG, CG, AT, AT, AT, CG, CG, CG, AT, AT, CG, CG, AT, AT, AT, CG, CG, AT, AT, CG, CG, CG, AT, AT, CG, CG, AT, AT, AT, CG, CG, CG, AT, AT, AT, CG, CG, AT, AT, CG, CG, CG, AT, AT, AT, CG, CG, CG, AT, AT, CG, CG, AT, AT, AT, CG, CG, CG, AT, AT, AT, CG, CG, AT, AT, CG, CG, CG, AT, AT, CG, CG, AT, AT, AT, CG, CG, AT, AT, CG, CG, CG, AT, AT, AT, CG, CG, CG, AT, AT, CG, CG, AT, AT, AT, CG, CG, AT, AT, CG, CG, CG, AT, AT, CG, CG, AT, AT, AT, CG, CG, CG, AT, AT, AT, CG, CG, AT, AT, CG, CG, CG, AT, AT, CG, CG, AT, AT, AT, CG, CG, AT, AT, CG, CG, CG, AT, AT, AT, CG, CG, CG, AT, AT, CG, CG, AT, AT, AT, CG, CG, CG, AT, AT, AT, CG, CG, AT, AT, CG, CG, CG, AT, AT, AT, CG, CG, CG, AT, AT, CG, CG, AT, AT, AT, CG, CG, AT, AT, CG, CG, CG, AT, AT, CG, CG, AT, AT, AT, CG, CG, CG, AT, AT, AT, CG, CG, AT, AT, CG, CG, CG, AT, AT, AT, CG, CG, CG, AT, AT, CG, CG, AT, AT, AT, CG, CG, CG, AT, AT, AT, CG, CG, AT, AT, CG, CG, CG, AT, AT, CG, CG, AT, AT, AT, CG, CG, AT, AT, CG, CG, CG, AT, AT, AT, CG, CG, CG, AT, AT, CG, CG, AT, AT, AT, CG, CG, CG, AT, AT, AT, CG, CG, AT, AT, CG, CG, CG, AT, AT, AT, CG, CG, CG, AT, AT, CG, CG, AT, AT, AT, CG, CG, AT, AT, CG, CG, CG, AT, AT, CG, CG, AT, AT, AT, CG, CG, CG, AT, AT, AT, CG, CG, AT, AT, CG, CG, CG, AT, AT, CG, CG, AT, AT, AT, CG, CG, AT, AT, CG, CG, CG, AT, AT, AT, CG, CG, CG, AT, AT, CG, CG, AT, AT, AT, CG, CG, AT, AT, CG, CG, CG, AT, AT, CG, CG, AT, AT, AT, CG, CG, CG, AT, AT, AT, CG, CG, AT, AT, CG, CG, CG, AT, AT, AT, CG, CG, CG, AT, AT, CG, CG, AT, AT, AT, CG, CG, CG, AT, AT, AT, CG, CG, AT, AT, CG, CG, CG, AT, AT, CG, CG, AT, AT, AT, CG, CG, AT, AT, CG, CG, CG, AT, AT, AT, CG, CG, CG, AT, AT, CG, CG, AT, AT, AT, CG, CG, AT, AT, CG, CG, CG, AT, AT, CG, CG, AT, AT, AT, CG, CG, CG, AT, AT, AT, CG, CG, AT, AT, CG, CG, CG, AT, AT, CG, CG, AT, AT, AT, CG, CG, AT, AT, CG, CG, CG, AT, AT, AT, CG, CG, CG, AT, AT, CG, CG, AT, AT, AT, CG, CG, CG, AT, AT, AT, CG, CG, AT, AT, CG, CG, CG, AT, AT, AT, CG, CG, CG, AT, AT, CG, CG, AT, AT, AT, CG, CG, AT, AT, CG, CG, CG, AT, AT, CG, CG, AT, AT, AT, CG, CG, CG, AT, AT, AT, CG, CG, AT, AT, CG, CG, CG, AT, AT, CG, CG, AT, AT, AT, CG, CG, AT, AT, CG, CG, CG, AT, AT, AT, CG, CG, CG, AT, AT, CG, CG, AT, AT, AT, CG, CG, AT, AT, CG, CG, CG, AT, AT, CG, CG, AT, AT, AT, CG, CG, CG, AT, AT, AT, CG, CG, AT, AT, CG, CG, CG, AT, AT, AT, CG, CG, CG, AT, AT, CG, CG, AT, AT, AT, CG, CG, CG, AT, AT, AT, CG, CG, AT, AT, CG, CG, CG, AT, AT, CG, CG, AT, AT, AT, CG, CG, AT, AT, CG, CG, CG, AT, AT, AT, CG, CG, CG, AT, AT, CG, CG, AT, AT, AT, CG, CG, CG, AT, AT, AT, CG, CG, AT, AT, CG, CG, CG, AT, AT, AT, CG, CG, CG, AT, AT, CG, CG, AT, AT, AT, CG, CG, AT, AT, CG, CG, CG, AT, AT, CG, CG, AT, AT, AT, CG, CG, CG, AT, AT, AT, CG, CG, AT, AT, CG, CG, CG, AT, AT, AT, CG, CG, CG, AT, AT, CG, CG, AT, AT, AT, CG, CG, CG, AT, AT, AT, CG, CG, AT, AT, CG, CG, CG, AT, AT, CG, CG, AT, AT, AT, CG, CG, AT, AT, CG, CG, CG, AT, AT, AT, CG, CG, CG, AT, AT, CG, CG, AT, AT, AT, CG, CG, AT, AT, CG, CG, CG, AT, AT, CG, CG, AT, AT, AT, CG, CG, CG, AT, AT, AT, CG, CG, AT, AT, CG, CG, CG, AT, AT, CG, CG, AT, AT, AT, CG, CG, AT, AT, CG, CG, CG, AT, AT, AT, CG, CG, CG, AT, AT, CG, CG, AT, AT, AT, CG, CG, CG, AT, AT, AT, CG, CG, AT, AT, CG, CG, CG, AT, AT, AT, CG, CG, CG, AT, AT, CG, CG, AT, AT, AT, CG, CG, AT, AT, CG, CG, CG, AT, AT, CG, CG, AT, AT, AT, CG, CG, CG, AT, AT, AT, CG, CG, AT, AT, CG, CG, CG, AT, AT, CG, CG, AT, AT, AT, CG, CG, AT, AT, CG, CG, CG, AT, AT, AT, CG, CG, CG, AT, AT, CG, CG, AT, AT, AT, CG, CG, AT, AT, CG, CG, CG, AT, AT, CG, CG, AT, AT, AT, CG, CG, CG, AT, AT, AT, CG, CG, AT, AT, CG, CG, CG, AT, AT, AT, CG, CG, CG, AT, AT, CG, CG, AT, AT, AT, CG, CG, CG, AT, AT, AT, CG, CG, AT, AT, CG, CG, CG, AT, AT, CG, CG, AT, AT, AT, CG, CG, AT, AT, CG, CG, CG, AT, AT, AT, CG, CG, CG, AT, AT, CG, CG, AT, AT, AT, CG, CG, AT, AT, CG, CG, CG, AT, AT, CG, CG, AT, AT, AT, CG, CG, CG, AT, AT, AT, CG, CG, AT, AT, CG, CG, CG, AT, AT, CG, CG, AT, AT, AT, CG, CG, AT, AT, CG, CG, CG, AT, AT, AT, CG, CG, CG, AT, AT, CG, CG, AT, AT, AT, CG, CG, CG, AT, AT, AT, CG, CG, AT, AT, CG, CG, CG, AT, AT, AT, CG, CG, CG, AT, AT, CG, CG, AT, AT, AT, CG, CG, AT, AT, CG, CG, CG, AT, AT, CG, CG, AT, AT, AT, CG, CG, CG, AT, AT, AT, CG, CG, AT, AT, CG, CG, CG, AT, AT, AT, CG, CG, CG, AT, AT, CG, CG, AT, AT, AT, CG, CG, CG, AT, AT, AT, CG, CG, AT, AT, CG, CG, CG, AT, AT, CG, CG, AT, AT, AT, CG, CG, AT, AT, CG, CG, CG, AT, AT, AT, CG, CG, CG, AT, AT, CG, CG, AT, AT, AT, CG, CG, CG, AT, AT, AT, CG, CG, AT, AT, CG, CG, CG, AT, AT, AT, CG, CG, CG, AT, AT, CG, CG, AT, AT, AT, CG, CG, AT, AT, CG, CG, CG, AT, AT, CG, CG, AT, AT, AT, CG, CG, CG, AT, AT, AT, CG, CG, AT, AT, CG, CG, CG, AT, AT, CG, CG, AT, AT, AT, CG, CG, AT, AT, CG, CG, CG, AT, AT, AT, CG, CG, CG, AT, AT, CG, CG, AT, AT, AT, CG, CG, AT, AT, CG, CG, CG, AT, AT, CG, CG, AT, AT, AT, CG, CG, CG, AT, AT, AT, CG, CG, AT, AT, CG, CG, CG, AT, AT, AT, CG, CG, CG, AT, AT, CG, CG, AT, AT, AT, CG, CG, CG, AT, AT, AT, CG, CG, AT, AT, CG, CG, CG, AT, AT, CG, CG, AT, AT, AT, CG, CG, AT, AT, CG, CG, CG, AT, AT, AT, CG, CG, CG, AT, AT, CG, CG, AT, AT, AT, CG, CG, AT, AT, CG, CG, CG, AT, AT, CG, CG, AT, AT, AT, CG, CG, CG, AT, AT, AT, CG, CG, AT, AT, CG, CG, CG, AT, AT, CG, CG, AT, AT, AT, CG, CG, AT, AT, CG, CG, CG, AT, AT, AT, CG, CG, CG, AT, AT, CG, CG, AT, AT, AT, CG, CG, CG, AT, AT, AT, CG, CG, AT, AT, CG, CG, CG, AT, AT, AT, CG, CG, CG, AT, AT, CG, CG, AT, AT, AT, CG, CG, AT, AT, CG, CG, CG, AT, AT, CG, CG, AT, AT, AT, CG, CG, CG, AT, AT, AT, CG, CG, AT, AT, CG, CG, CG, AT, AT, CG, CG, AT, AT, AT, CG, CG, AT, AT, CG, CG, CG, AT, AT, AT, CG, CG, CG, AT, AT, CG, CG, AT, AT, AT, CG, CG, AT, AT, CG, CG, CG, AT, AT, CG, CG, AT, AT, AT, CG, CG, CG, AT, AT, AT, CG, CG, AT, AT, CG, CG, CG, AT, AT, AT, CG, CG, CG, AT, AT, CG, CG, AT, AT, AT, CG, CG, CG, AT, AT, AT, CG, CG, AT, AT, CG, CG, CG, AT, AT, CG, CG, AT, AT, AT, CG, CG, AT, AT, CG, CG, CG, AT, AT, AT, CG, CG, CG, AT, AT, CG, CG, AT, AT, AT, CG, CG, CG, AT, AT, AT, CG, CG, AT, AT, CG, CG, CG, AT, AT, AT, CG, CG, CG, AT, AT, CG, CG, AT, AT, AT, CG, CG, AT, AT, CG, CG, CG, AT, AT, CG, CG, AT, AT, AT, CG, CG, CG, AT, AT, AT, CG, CG, AT, AT, CG, CG, CG, AT, AT, AT, CG, CG, CG, AT, AT, CG, CG, AT, AT, AT, CG, CG, CG, AT, AT, AT, CG, CG, AT, AT, CG, CG, CG, AT, AT, CG, CG, AT, AT, AT, CG, CG, AT, AT, CG, CG, CG, AT, AT, AT, CG, CG, CG, AT, AT, CG, CG, AT, AT, AT, CG, CG, AT, AT, CG, CG, CG, AT, AT, CG, CG, AT, AT, AT, CG, CG, CG, AT, AT, AT, CG, CG, AT, AT, CG, CG, CG, AT, AT, CG, CG, AT, AT, AT, CG, CG, AT, AT, CG, CG, CG, AT, AT, AT, CG, CG, CG, AT, AT, CG, CG, AT, AT, AT, CG, CG, CG, AT, AT, AT, CG, CG, AT, AT, CG, CG, CG, AT, AT, AT, CG, CG, CG, AT, AT, CG, CG, AT, AT, AT, CG, CG, AT, AT, CG, CG, CG, AT, AT, CG, CG, AT, AT, AT, CG, CG, CG, AT, AT, AT, CG, CG, AT, AT, CG, CG, CG, AT, AT, AT, CG, CG, CG, AT, AT, CG, CG, AT, AT, AT, CG, CG, CG, AT, AT, AT, CG, CG, AT, AT, CG, CG, CG, AT, AT, CG, CG, AT, AT, AT, CG, CG, AT, AT, CG, CG, CG, AT, AT, AT, CG, CG, CG, AT, AT, CG, CG, AT, AT, AT, CG, CG, CG, AT, AT, AT, CG, CG, AT, AT, CG, CG, CG, AT, AT, AT, CG, CG, CG, AT, AT, CG, CG, AT, AT, AT, CG, CG, AT, AT, CG, CG, CG, AT, AT, CG, CG, AT, AT, AT, CG, CG, CG, AT, AT, AT, CG, CG, AT, AT, CG, CG, CG, AT, AT, CG, CG, AT, AT, AT, CG, CG, AT, AT, CG, CG, CG, AT, AT, AT, CG, CG, CG, AT, AT, CG, CG, AT, AT, AT, CG, CG, AT, AT, CG, CG, CG, AT, AT, CG, CG, AT, AT, AT, CG, CG, CG, AT, AT, AT, CG, CG, AT, AT, CG, CG, CG, AT, AT, AT, CG, CG, CG, AT, AT, CG, CG, AT, AT, AT, CG, CG, CG, AT, AT, AT, CG, CG, AT, AT, CG, CG, CG, AT, AT, CG, CG, AT, AT, AT, CG, CG, AT, AT, CG, CG, CG, AT, AT, AT, CG, CG, CG, AT, AT, CG, CG, AT, AT, AT, CG, CG, CG, AT, AT, AT, CG, CG, AT, AT, CG, CG, CG, AT, AT, AT, CG, CG, CG, AT, AT, CG, CG, AT, AT, AT, CG, CG, AT, AT, CG, CG, CG, AT, AT, CG, CG, AT, AT, AT, CG, CG, CG, AT, AT, AT, CG, CG, AT, AT, CG, CG, CG, AT, AT, AT, CG, CG, CG, AT, AT, CG, CG, AT, AT, AT, CG, CG, CG, AT, AT, AT, CG, CG, AT, AT, CG, CG, CG, AT, AT, CG, CG, AT, AT, AT, CG, CG, AT, AT, CG, CG, CG, AT, AT, AT, CG, CG, CG, AT, AT, CG, CG, AT, AT, AT, CG, CG, AT, AT, CG, CG, CG, AT, AT, CG, CG, AT, AT, AT, CG, CG, CG, AT, AT, AT, CG, CG, AT, AT, CG, CG, CG, AT, AT, CG, CG, AT, AT, AT, CG, CG, AT, AT, CG, CG, CG, AT, AT, AT, CG, CG, CG, AT, AT, CG, CG, AT, AT, AT, CG, CG, CG, AT, AT, AT, CG, CG, AT, AT, CG, CG, CG, AT, AT, AT, CG, CG, CG, AT, AT, CG, CG, AT, AT, AT, CG, CG, AT, AT, CG, CG, CG, AT, AT, CG, CG, AT, AT, AT, CG, CG, CG, AT, AT, AT, CG, CG, AT, AT, CG, CG, CG, AT, AT, CG, CG, AT, AT, AT, CG, CG, AT, AT, CG, CG, CG, AT |
| Thue-Morse 3 | AT, AT, AT, CG, CG, CG, CG, CG, CG, AT, AT, AT, CG, CG, CG, AT, AT, AT, AT, AT, AT, CG, CG, CG, CG, CG, CG, AT, AT, AT, AT, AT, AT, CG, CG, CG, AT, AT, AT, CG, CG, CG, CG, CG, CG, AT, AT, AT, CG, CG, CG, AT, AT, AT, AT, AT, AT, CG, CG, CG, AT, AT, AT, CG, CG, CG, CG, CG, CG, AT, AT, AT, AT, AT, AT, CG, CG, CG, CG, CG, CG, AT, AT, AT, CG, CG, CG, AT, AT, AT, AT, AT, AT, CG, CG, CG, CG, CG, CG, AT, AT, AT, AT, AT, AT, CG, CG, CG, AT, AT, AT, CG, CG, CG, CG, CG, CG, AT, AT, AT, AT, AT, AT, CG, CG, CG, CG, CG, CG, AT, AT, AT, CG, CG, CG, AT, AT, AT, AT, AT, AT, CG, CG, CG, AT, AT, AT, CG, CG, CG, CG, CG, CG, AT, AT, AT, CG, CG, CG, AT, AT, AT, AT, AT, AT, CG, CG, CG, CG, CG, CG, AT, AT, AT, AT, AT, AT, CG, CG, CG, AT, AT, AT, CG, CG, CG, CG, CG, CG, AT, AT, AT, CG, CG, CG, AT, AT, AT, AT, AT, AT, CG, CG, CG, AT, AT, AT, CG, CG, CG, CG, CG, CG, AT, AT, AT, AT, AT, AT, CG, CG, CG, CG, CG, CG, AT, AT, AT, CG, CG, CG, AT, AT, AT, AT, AT, AT, CG, CG, CG, AT, AT, AT, CG, CG, CG, CG, CG, CG, AT, AT, AT, CG, CG, CG, AT, AT, AT, AT, AT, AT, CG, CG, CG, CG, CG, CG, AT, AT, AT, AT, AT, AT, CG, CG, CG, AT, AT, AT, CG, CG, CG, CG, CG, CG, AT, AT, AT, AT, AT, AT, CG, CG, CG, CG, CG, CG, AT, AT, AT, CG, CG, CG, AT, AT, AT, AT, AT, AT, CG, CG, CG, CG, CG, CG, AT, AT, AT, AT, AT, AT, CG, CG, CG, AT, AT, AT, CG, CG, CG, CG, CG, CG, AT, AT, AT, CG, CG, CG, AT, AT, AT, AT, AT, AT, CG, CG, CG, AT, AT, AT, CG, CG, CG, CG, CG, CG, AT, AT, AT, AT, AT, AT, CG, CG, CG, CG, CG, CG, AT, AT, AT, CG, CG, CG, AT, AT, AT, AT, AT, AT, CG, CG, CG, CG, CG, CG, AT, AT, AT, AT, AT, AT, CG, CG, CG, AT, AT, AT, CG, CG, CG, CG, CG, CG, AT, AT, AT, AT, AT, AT, CG, CG, CG, CG, CG, CG, AT, AT, AT, CG, CG, CG, AT, AT, AT, AT, AT, AT, CG, CG, CG, AT, AT, AT, CG, CG, CG, CG, CG, CG, AT, AT, AT, CG, CG, CG, AT, AT, AT, AT, AT, AT, CG, CG, CG, CG, CG, CG, AT, AT, AT, AT, AT, AT, CG, CG, CG, AT, AT, AT, CG, CG, CG, CG, CG, CG, AT, AT, AT, AT, AT, AT, CG, CG, CG, CG, CG, CG, AT, AT, AT, CG, CG, CG, AT, AT, AT, AT, AT, AT, CG, CG, CG, CG, CG, CG, AT, AT, AT, AT, AT, AT, CG, CG, CG, AT, AT, AT, CG, CG, CG, CG, CG, CG, AT, AT, AT, CG, CG, CG, AT, AT, AT, AT, AT, AT, CG, CG, CG, AT, AT, AT, CG, CG, CG, CG, CG, CG, AT, AT, AT, AT, AT, AT, CG, CG, CG, CG, CG, CG, AT, AT, AT, CG, CG, CG, AT, AT, AT, AT, AT, AT, CG, CG, CG, AT, AT, AT, CG, CG, CG, CG, CG, CG, AT, AT, AT, CG, CG, CG, AT, AT, AT, AT, AT, AT, CG, CG, CG, CG, CG, CG, AT, AT, AT, AT, AT, AT, CG, CG, CG, AT, AT, AT, CG, CG, CG, CG, CG, CG, AT, AT, AT, CG, CG, CG, AT, AT, AT, AT, AT, AT, CG, CG, CG, AT, AT, AT, CG, CG, CG, CG, CG, CG, AT, AT, AT, AT, AT, AT, CG, CG, CG, CG, CG, CG, AT, AT, AT, CG, CG, CG, AT, AT, AT, AT, AT, AT, CG, CG, CG, CG, CG, CG, AT, AT, AT, AT, AT, AT, CG, CG, CG, AT, AT, AT, CG, CG, CG, CG, CG, CG, AT, AT, AT, AT, AT, AT, CG, CG, CG, CG, CG, CG, AT, AT, AT, CG, CG, CG, AT, AT, AT, AT, AT, AT, CG, CG, CG, AT, AT, AT, CG, CG, CG, CG, CG, CG, AT, AT, AT, CG, CG, CG, AT, AT, AT, AT, AT, AT, CG, CG, CG, CG, CG, CG, AT, AT, AT, AT, AT, AT, CG, CG, CG, AT, AT, AT, CG, CG, CG, CG, CG, CG, AT, AT, AT, CG, CG, CG, AT, AT, AT, AT, AT, AT, CG, CG, CG, AT, AT, AT, CG, CG, CG, CG, CG, CG, AT, AT, AT, AT, AT, AT, CG, CG, CG, CG, CG, CG, AT, AT, AT, CG, CG, CG, AT, AT, AT, AT, AT, AT, CG, CG, CG, AT, AT, AT, CG, CG, CG, CG, CG, CG, AT, AT, AT, CG, CG, CG, AT, AT, AT, AT, AT, AT, CG, CG, CG, CG, CG, CG, AT, AT, AT, AT, AT, AT, CG, CG, CG, AT, AT, AT, CG, CG, CG, CG, CG, CG, AT, AT, AT, AT, AT, AT, CG, CG, CG, CG, CG, CG, AT, AT, AT, CG, CG, CG, AT, AT, AT, AT, AT, AT, CG, CG, CG, CG, CG, CG, AT, AT, AT, AT, AT, AT, CG, CG, CG, AT, AT, AT, CG, CG, CG, CG, CG, CG, AT, AT, AT, CG, CG, CG, AT, AT, AT, AT, AT, AT, CG, CG, CG, AT, AT, AT, CG, CG, CG, CG, CG, CG, AT, AT, AT, AT, AT, AT, CG, CG, CG, CG, CG, CG, AT, AT, AT, CG, CG, CG, AT, AT, AT, AT, AT, AT, CG, CG, CG, AT, AT, AT, CG, CG, CG, CG, CG, CG, AT, AT, AT, CG, CG, CG, AT, AT, AT, AT, AT, AT, CG, CG, CG, CG, CG, CG, AT, AT, AT, AT, AT, AT, CG, CG, CG, AT, AT, AT, CG, CG, CG, CG, CG, CG, AT, AT, AT, CG, CG, CG, AT, AT, AT, AT, AT, AT, CG, CG, CG, AT, AT, AT, CG, CG, CG, CG, CG, CG, AT, AT, AT, AT, AT, AT, CG, CG, CG, CG, CG, CG, AT, AT, AT, CG, CG, CG, AT, AT, AT, AT, AT, AT, CG, CG, CG, CG, CG, CG, AT, AT, AT, AT, AT, AT, CG, CG, CG, AT, AT, AT, CG, CG, CG, CG, CG, CG, AT, AT, AT, AT, AT, AT, CG, CG, CG, CG, CG, CG, AT, AT, AT, CG, CG, CG, AT, AT, AT, AT, AT, AT, CG, CG, CG, AT, AT, AT, CG, CG, CG, CG, CG, CG, AT, AT, AT, CG, CG, CG, AT, AT, AT, AT, AT, AT, CG, CG, CG, CG, CG, CG, AT, AT, AT, AT, AT, AT, CG, CG, CG, AT, AT, AT, CG, CG, CG, CG, CG, CG, AT, AT, AT, AT, AT, AT, CG, CG, CG, CG, CG, CG, AT, AT, AT, CG, CG, CG, AT, AT, AT, AT, AT, AT, CG, CG, CG, CG, CG, CG, AT, AT, AT, AT, AT, AT, CG, CG, CG, AT, AT, AT, CG, CG, CG, CG, CG, CG, AT, AT, AT, CG, CG, CG, AT, AT, AT, AT, AT, AT, CG, CG, CG, AT, AT, AT, CG, CG, CG, CG, CG, CG, AT, AT, AT, AT, AT, AT, CG, CG, CG, CG, CG, CG, AT, AT, AT, CG, CG, CG, AT, AT, AT, AT, AT, AT, CG, CG, CG, CG, CG, CG, AT, AT, AT, AT, AT, AT, CG, CG, CG, AT, AT, AT, CG, CG, CG, CG, CG, CG, AT, AT, AT, AT, AT, AT, CG, CG, CG, CG, CG, CG, AT, AT, AT, CG, CG, CG, AT, AT, AT, AT, AT, AT, CG, CG, CG, AT, AT, AT, CG, CG, CG, CG, CG, CG, AT, AT, AT, CG, CG, CG, AT, AT, AT, AT, AT, AT, CG, CG, CG, CG, CG, CG, AT, AT, AT, AT, AT, AT, CG, CG, CG, AT, AT, AT, CG, CG, CG, CG, CG, CG, AT, AT, AT, CG, CG, CG, AT, AT, AT, AT, AT, AT, CG, CG, CG, AT, AT, AT, CG, CG, CG, CG, CG, CG, AT, AT, AT, AT, AT, AT, CG, CG, CG, CG, CG, CG, AT, AT, AT, CG, CG, CG, AT, AT, AT, AT, AT, AT, CG, CG, CG, AT, AT, AT, CG, CG, CG, CG, CG, CG, AT, AT, AT, CG, CG, CG, AT, AT, AT, AT, AT, AT, CG, CG, CG, CG, CG, CG, AT, AT, AT, AT, AT, AT, CG, CG, CG, AT, AT, AT, CG, CG, CG, CG, CG, CG, AT, AT, AT, AT, AT, AT, CG, CG, CG, CG, CG, CG, AT, AT, AT, CG, CG, CG, AT, AT, AT, AT, AT, AT, CG, CG, CG, CG, CG, CG, AT, AT, AT, AT, AT, AT, CG, CG, CG, AT, AT, AT, CG, CG, CG, CG, CG, CG, AT, AT, AT, CG, CG, CG, AT, AT, AT, AT, AT, AT, CG, CG, CG, AT, AT, AT, CG, CG, CG, CG, CG, CG, AT, AT, AT, AT, AT, AT, CG, CG, CG, CG, CG, CG, AT, AT, AT, CG, CG, CG, AT, AT, AT, AT, AT, AT, CG, CG, CG, CG, CG, CG, AT, AT, AT, AT, AT, AT, CG, CG, CG, AT, AT, AT, CG, CG, CG, CG, CG, CG, AT, AT, AT, AT, AT, AT, CG, CG, CG, CG, CG, CG, AT, AT, AT, CG, CG, CG, AT, AT, AT, AT, AT, AT, CG, CG, CG, AT, AT, AT, CG, CG, CG, CG, CG, CG, AT, AT, AT, CG, CG, CG, AT, AT, AT, AT, AT, AT, CG, CG, CG, CG, CG, CG, AT, AT, AT, AT, AT, AT, CG, CG, CG, AT, AT, AT, CG, CG, CG, CG, CG, CG, AT, AT, AT, AT, AT, AT, CG, CG, CG, CG, CG, CG, AT, AT, AT, CG, CG, CG, AT, AT, AT, AT, AT, AT, CG, CG, CG, CG, CG, CG, AT, AT, AT, AT, AT, AT, CG, CG, CG, AT, AT, AT, CG, CG, CG, CG, CG, CG, AT, AT, AT, CG, CG, CG, AT, AT, AT, AT, AT, AT, CG, CG, CG, AT, AT, AT, CG, CG, CG, CG, CG, CG, AT, AT, AT, AT, AT, AT, CG, CG, CG, CG, CG, CG, AT, AT, AT, CG, CG, CG, AT, AT, AT, AT, AT, AT, CG, CG, CG, AT, AT, AT, CG, CG, CG, CG, CG, CG, AT, AT, AT, CG, CG, CG, AT, AT, AT, AT, AT, AT, CG, CG, CG, CG, CG, CG, AT, AT, AT, AT, AT, AT, CG, CG, CG, AT, AT, AT, CG, CG, CG, CG, CG, CG, AT, AT, AT, CG, CG, CG, AT, AT, AT, AT, AT, AT, CG, CG, CG, AT, AT, AT, CG, CG, CG, CG, CG, CG, AT, AT, AT, AT, AT, AT, CG, CG, CG, CG, CG, CG, AT, AT, AT, CG, CG, CG, AT, AT, AT, AT, AT, AT, CG, CG, CG, CG, CG, CG, AT, AT, AT, AT, AT, AT, CG, CG, CG, AT, AT, AT, CG, CG, CG, CG, CG, CG, AT, AT, AT, AT, AT, AT, CG, CG, CG, CG, CG, CG, AT, AT, AT, CG, CG, CG, AT, AT, AT, AT, AT, AT, CG, CG, CG, AT, AT, AT, CG, CG, CG, CG, CG, CG, AT, AT, AT, CG, CG, CG, AT, AT, AT, AT, AT, AT, CG, CG, CG, CG, CG, CG, AT, AT, AT, AT, AT, AT, CG, CG, CG, AT, AT, AT, CG, CG, CG, CG, CG, CG, AT, AT, AT, AT, AT, AT, CG, CG, CG, CG, CG, CG, AT, AT, AT, CG, CG, CG, AT, AT, AT, AT, AT, AT, CG, CG, CG, CG, CG, CG, AT, AT, AT, AT, AT, AT, CG, CG, CG, AT, AT, AT, CG, CG, CG, CG, CG, CG, AT, AT, AT, CG, CG, CG, AT, AT, AT, AT, AT, AT, CG, CG, CG, AT, AT, AT, CG, CG, CG, CG, CG, CG, AT, AT, AT, AT, AT, AT, CG, CG, CG, CG, CG, CG, AT, AT, AT, CG, CG, CG, AT, AT, AT, AT, AT, AT, CG, CG, CG, CG, CG, CG, AT, AT, AT, AT, AT, AT, CG, CG, CG, AT, AT, AT, CG, CG, CG, CG, CG, CG, AT, AT, AT, AT, AT, AT, CG, CG, CG, CG, CG, CG, AT, AT, AT, CG, CG, CG, AT, AT, AT, AT, AT, AT, CG, CG, CG, AT, AT, AT, CG, CG, CG, CG, CG, CG, AT, AT, AT, CG, CG, CG, AT, AT, AT, AT, AT, AT, CG, CG, CG, CG, CG, CG, AT, AT, AT, AT, AT, AT, CG, CG, CG, AT, AT, AT, CG, CG, CG, CG, CG, CG, AT, AT, AT, CG, CG, CG, AT, AT, AT, AT, AT, AT, CG, CG, CG, AT, AT, AT, CG, CG, CG, CG, CG, CG, AT, AT, AT, AT, AT, AT, CG, CG, CG, CG, CG, CG, AT, AT, AT, CG, CG, CG, AT, AT, AT, AT, AT, AT, CG, CG, CG, AT, AT, AT, CG, CG, CG, CG, CG, CG, AT, AT, AT, CG, CG, CG, AT, AT, AT, AT, AT, AT, CG, CG, CG, CG, CG, CG, AT, AT, AT, AT, AT, AT, CG, CG, CG, AT, AT, AT, CG, CG, CG, CG, CG, CG, AT, AT, AT, AT, AT, AT, CG, CG, CG, CG, CG, CG, AT, AT, AT, CG, CG, CG, AT, AT, AT, AT, AT, AT, CG, CG, CG, CG, CG, CG, AT, AT, AT, AT, AT, AT, CG, CG, CG, AT, AT, AT, CG, CG, CG, CG, CG, CG, AT, AT, AT, CG, CG, CG, AT, AT, AT, AT, AT, AT, CG, CG, CG, AT, AT, AT, CG, CG, CG, CG, CG, CG, AT, AT, AT, AT, AT, AT, CG, CG, CG, CG, CG, CG, AT, AT, AT, CG, CG, CG, AT, AT, AT, AT, AT, AT, CG, CG, CG, AT, AT, AT, CG, CG, CG, CG, CG, CG, AT, AT, AT, CG, CG, CG, AT, AT, AT, AT, AT, AT, CG, CG, CG, CG, CG, CG, AT, AT, AT, AT, AT, AT, CG, CG, CG, AT, AT, AT, CG, CG, CG, CG, CG, CG, AT, AT, AT, CG, CG, CG, AT, AT, AT, AT, AT, AT, CG, CG, CG, AT, AT, AT, CG, CG, CG, CG, CG, CG, AT, AT, AT, AT, AT, AT, CG, CG, CG, CG, CG, CG, AT, AT, AT, CG, CG, CG, AT, AT, AT, AT, AT, AT, CG, CG, CG, CG, CG, CG, AT, AT, AT, AT, AT, AT, CG, CG, CG, AT, AT, AT, CG, CG, CG, CG, CG, CG, AT, AT, AT, AT, AT, AT, CG, CG, CG, CG, CG, CG, AT, AT, AT, CG, CG, CG, AT, AT, AT, AT, AT, AT, CG, CG, CG, AT, AT, AT, CG, CG, CG, CG, CG, CG, AT, AT, AT, CG, CG, CG, AT, AT, AT, AT, AT, AT, CG, CG, CG, CG, CG, CG, AT, AT, AT, AT, AT, AT, CG, CG, CG, AT, AT, AT, CG, CG, CG, CG, CG, CG, AT, AT, AT, CG, CG, CG, AT, AT, AT, AT, AT, AT, CG, CG, CG, AT, AT, AT, CG, CG, CG, CG, CG, CG, AT, AT, AT, AT, AT, AT, CG, CG, CG, CG, CG, CG, AT, AT, AT, CG, CG, CG, AT, AT, AT, AT, AT, AT, CG, CG, CG, AT, AT, AT, CG, CG, CG, CG, CG, CG, AT, AT, AT, CG, CG, CG, AT, AT, AT, AT, AT, AT, CG, CG, CG, CG, CG, CG, AT, AT, AT, AT, AT, AT, CG, CG, CG, AT, AT, AT, CG, CG, CG, CG, CG, CG, AT, AT, AT, AT, AT, AT, CG, CG, CG, CG, CG, CG, AT, AT, AT, CG, CG, CG, AT, AT, AT, AT, AT, AT, CG, CG, CG, CG, CG, CG, AT, AT, AT, AT, AT, AT, CG, CG, CG, AT, AT, AT, CG, CG, CG, CG, CG, CG, AT, AT, AT, CG, CG, CG, AT, AT, AT, AT, AT, AT, CG, CG, CG, AT, AT, AT, CG, CG, CG, CG, CG, CG, AT, AT, AT, AT, AT, AT, CG, CG, CG, CG, CG, CG, AT, AT, AT, CG, CG, CG, AT, AT, AT, AT, AT, AT, CG, CG, CG, CG, CG, CG, AT, AT, AT, AT, AT, AT, CG, CG, CG, AT, AT, AT, CG, CG, CG, CG, CG, CG, AT, AT, AT, AT, AT, AT, CG, CG, CG, CG, CG, CG, AT, AT, AT, CG, CG, CG, AT, AT, AT, AT, AT, AT, CG, CG, CG, AT, AT, AT, CG, CG, CG, CG, CG, CG, AT, AT, AT, CG, CG, CG, AT, AT, AT, AT, AT, AT, CG, CG, CG, CG, CG, CG, AT, AT, AT, AT, AT, AT, CG, CG, CG, AT, AT, AT, CG, CG, CG, CG, CG, CG, AT, AT, AT, AT, AT, AT, CG, CG, CG, CG, CG, CG, AT, AT, AT, CG, CG, CG, AT, AT, AT, AT, AT, AT, CG, CG, CG, CG, CG, CG, AT, AT, AT, AT, AT, AT, CG, CG, CG, AT, AT, AT, CG, CG, CG, CG, CG, CG, AT, AT, AT, CG, CG, CG, AT, AT, AT, AT, AT, AT, CG, CG, CG, AT, AT, AT, CG, CG, CG, CG, CG, CG, AT, AT, AT, AT, AT, AT, CG, CG, CG, CG, CG, CG, AT, AT, AT, CG, CG, CG, AT, AT, AT, AT, AT, AT, CG, CG, CG, AT, AT, AT, CG, CG, CG, CG, CG, CG, AT, AT, AT, CG, CG, CG, AT, AT, AT, AT, AT, AT, CG, CG, CG, CG, CG, CG, AT, AT, AT, AT, AT, AT, CG, CG, CG, AT, AT, AT, CG, CG, CG, CG, CG, CG, AT, AT, AT, CG, CG, CG, AT, AT, AT, AT, AT, AT, CG, CG, CG, AT, AT, AT, CG, CG, CG, CG, CG, CG, AT, AT, AT, AT, AT, AT, CG, CG, CG, CG, CG, CG, AT, AT, AT, CG, CG, CG, AT, AT, AT, AT, AT, AT, CG, CG, CG, CG, CG, CG, AT, AT, AT, AT, AT, AT, CG, CG, CG, AT, AT, AT, CG, CG, CG, CG, CG, CG, AT, AT, AT |
| Thue-morse 4 | AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, CG, CG, CG, CG, AT, AT, AT, AT, AT, AT, AT, AT, CG, CG, CG, CG, AT, AT, AT, AT, CG, CG, CG, CG, CG, CG, CG, CG, AT, AT, AT, AT, AT, AT, AT, AT, CG, CG, CG, CG, CG, CG, CG, CG, AT, AT, AT, AT, CG, CG, CG, CG, AT, AT, AT, AT, AT, AT, AT, AT, CG, CG, CG, CG, CG, CG, CG, CG, AT, AT, AT, AT, AT, AT, AT, AT, CG, CG, CG, CG, AT, AT, AT, AT, CG, CG, CG, CG |

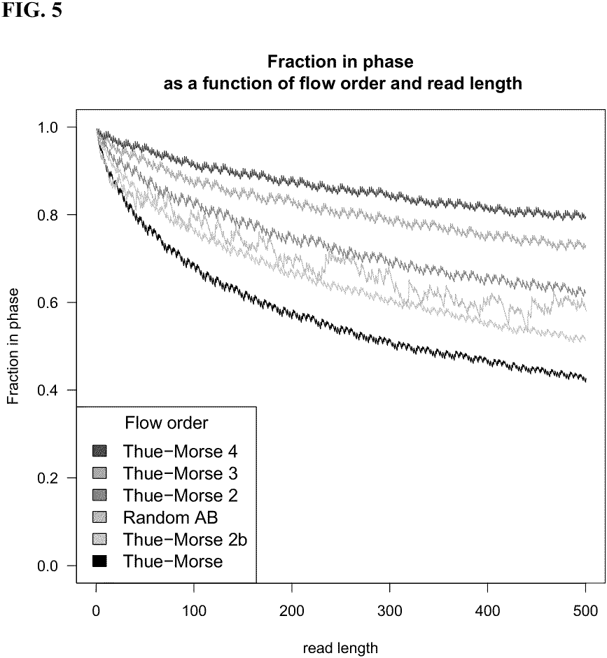

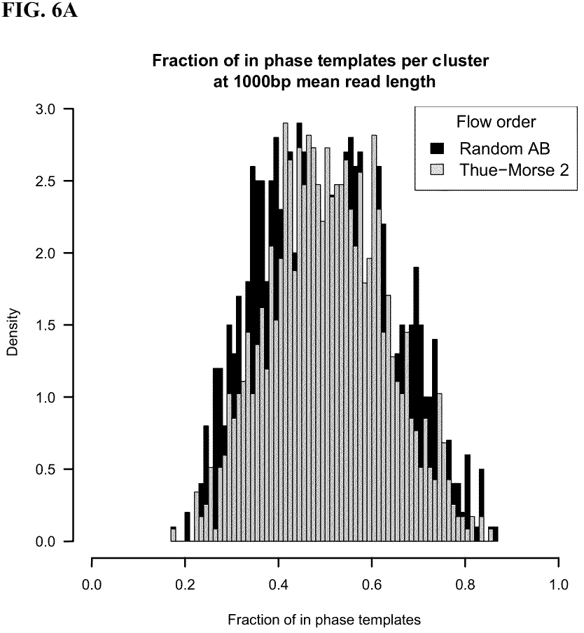

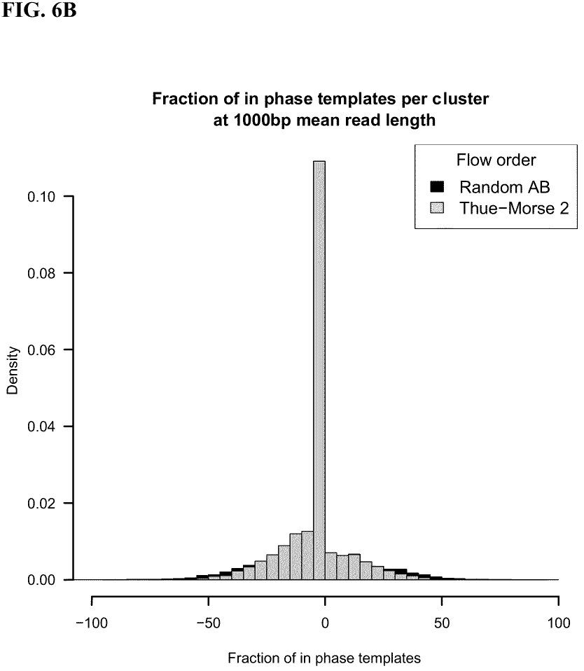

The Thue-Morse 2 sequence outperforms Random AB with respect to phase protection and overall base quality, while having a comparable sequencing efficiency and reduced variance in base quality per cycle (FIG. 5 and Table 1). Thue-Morse 2b shows reduced phase protection compared to Thue-Morse 2 but greater sequencing efficiency. Thue-Morse 3 and 4 sequences are able to exceed the phase protection of the Random AB order at the expense of sequencing efficiency. At a read length of 1000bp, Thue-Morse 2 and Random AB sequences show a similar distribution in the fraction of in phase templates per cluster (FIG. 6A), although Thue-Morse 2 preserves a higher average fraction of in phase templates per cycle (0.622 vs 0.648, respectively). The Thue-Morse 2 and Random AB sequences produce a similar distribution of phasing offsets, with neither appearing to synchronize out of phase templates (FIG. 6B). Importantly, Thue-Morse 2, 2b, 3, and 4 sequences consist entirely of at least two consecutive flows of the same solution. This property is advantageous for downstream signal interpretation as one may compare signal measurements from consecutive flows of the same solution to identify mistakes in base calling, thereby enabling ‘self-correction’ of the resultant read sequence without prior knowledge of the template sequence. Taken together, these results indicate Thue-Morse derived sequences provide advantageous flow orders for a two-solution DNA sequencing paradigm.

### Developing Base Calling Algorithms for Use With Alternative Flow Sequencing Data

Unlike traditional four nucleotide flow orderings, a flow order employing fewer than four nucleotides per flow is expected to yield a mixture of illuminated template clusters, where the dominating in phase template population incorporates one of the flowed nucleotides, and dark clusters, where extension of the dominating in phase template population requires a nucleotide that is absent from the flowed pool of nucleotides. Consequently, alternative flow orders effectively enable multiple measurements of a single nucleotide incorporation event: a direct measurement of the signal tag from a template nucleotide incorporation, resulting in an illuminated cluster, and an indirect detection of a template nucleotide incorporation, where one observes an absence of nucleotide incorporation, corresponding to a dark cluster. In the latter case, one knows which nucleotide(s) were absent from the cycle and thus one can infer the identity of the next base to be incorporated into the cluster.

This unique attribute of alternative flow orders may be exploited to enable higher sequencing accuracy than is attainable via a traditional four nucleotide flow system. In one embodiment, a machine learning base calling algorithm is trained to leverage the information conveyed by dark and illuminated clusters. For each sequencing cycle, the machine learning algorithm receives as input the nucleotide signal intensities for each of the clusters on the flow cell, the nucleotides that were flowed in that cycle and the previous cycles, and the base calls for the previous cycles for the same cluster (e.g., dA, dT, dC, dG, or ‘D’ for dark). Using the intensities of the signals for the current cycle, the algorithm determines whether a given cluster is illuminated or dark in that cycle. If the cluster is determined to be illuminated, the algorithm examines the state of the cluster in the previous cycle. If in the previous cycle the cluster was classified as dark, the algorithm weighs the current cycle base calling to favor nucleotide(s) that were missing during that dark cycle. If the previous and current cycles were identified as dark but the nucleotides flowed differed between the cycles such that one would expect at least one illuminated cycle, then the identified cluster state is incompatible with the flow order, and the signals from the conflicting cycles may be reinterpreted to be compatible with the flow order. Optionally, such an incompatibility may be used to reduce the estimated quality of the cluster sequence. More broadly, this latter process enables one to use expectations relating to the temporal ordering of illuminated and dark clusters as a quality control measurement. In some implementations, the machine learning algorithm examines the cluster state during the preceding cycle, but also the state during one or more of the earlier cycles.

In one embodiment, training of a machine learning model may be accomplished by conducting a sequencing experiment where a given number of initial sequencing cycles are performed using a default four nucleotide flow to generate a sequence that is readily interpretable by a four-nucleotide flow analysis algorithm. These cycles are followed by additional cycles where an alternative flow order is implemented. The sequence derived from the four nucleotide flows may be used to map the template sequence to a reference genome and infer the subsequent template sequence. Thus, the initial cycles should be numerous enough to generate a sequence kmer that enables unambiguous mapping of the template sequence to the reference genome. In some embodiments, the number of four nucleotide cycles is approximately 20, the number of alternative flow cycles is approximately 50-100 and the reference genome is the salmonella genome. In other embodiments, the training may be performed using a well characterized human genome, for example the specimen NA12878 (Zook J. et al., Nat. Biotechnol. 32, 246-251 (2014)), or combinations of genomes deriving from different organisms and differing with respect to GC content and sequence complexity (e.g., repetitive elements, low complexity sequence, etc.). Using knowledge of the template sequence as well as the flow ordering, one creates the true incorporation sequence by including the template nucleotide when the flow order permits incorporation and the no incorporation event (termed dark) when the template’s next nucleotide is not in the current flow.

In experiments corresponding to alternative flow cycles, a previously trained alternative flow basecaller may be used to generate a prospective basecall sequence. By removing the predicted dark cycles and aligning the remaining cycles for each template to the appropriate reference genome, the template’s true sequence may be determined. In the case where all template sequences cannot be unambiguously determined, one may do several rounds of model training where each successive model is able to generate a more accurate training set for the experiment. Additionally, one can enforce self-consistency within the training dataset. For example, if a cluster is identified as dark following flow of nucleotide solution ‘A’, then if the next flow also consists of solution ‘A’ one should also identify the cluster as dark. Similarly, if a cluster is identified as dark following a flow of solution ‘A’, then it must be identified as having an incorporation following a flow of the complementary solution ‘B’. Rules of this type can be enforced when creating the dataset to ensure the model learns a self-consistent basecalling algorithm. Additionally, self-consistency metrics can be used on basecalling outputs in the field to ensure that the model is well-calibrated to the user’s data. Outputs failing to meet a minimum self-consistency threshold can trigger, for instance, a request to access the user’s raw data to add it to the basecaller training set.

One can train a basecalling model on a dataset of templates, where each template contains three sequences of data: the signal intensities, the alternative flow order, and the incorporation history (A, C, T, G, no incorporation). In some embodiments, the dataset will correspond to a mixture of numerous experiments, where each experiment may have differing conditions (such as species or alternative flow protocols). Using standard supervised learning techniques, one can train models where the inputs are: a sequence of one or more cycles of signal intensities, a sequence of the cycle numbers, and a sequence of the nucleotides flowed and the output labels are the sequence of one or more cycles of the true incorporation history. The model may be a generic neural network architecture capable of accommodating the sequence of inputs and outputs, some embodiments may require a specified sequence length whereas other may handle sequences of variable lengths. In one embodiment, the sequence length is 30, the model includes recurrent, dense, and softmax network layers, and the loss function optimized is categorical cross entropy. In this example, the dataset is separated into an 80%, 20% split, where the model is trained on the 80% of data. To evaluate performance, the model may be validated on the 20% of data that the model was not trained on. Once trained and validated, the algorithm may finally be employed to analyze data derived exclusively from application of the alternative flow. Specific methods for training such machine learning models are known to those knowledgeable in the art and may include application of Python-based machine learning libraries such as Scikit-learn, Theano, TensorFlow, Keras and PyTorch, among others.

### Example 2. Evaluating Phase Protection by Sequencing of Known Templates

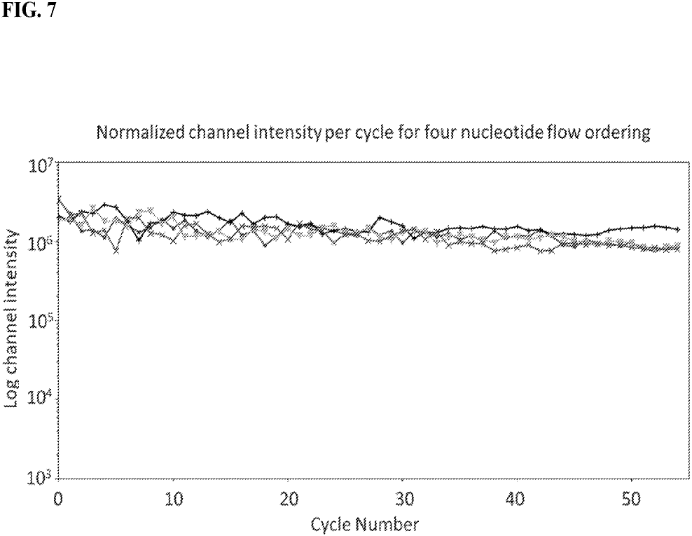

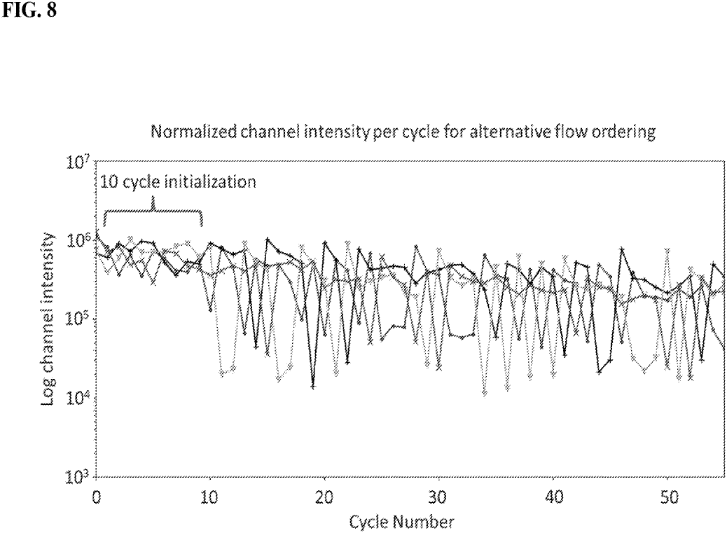

To validate the results of the simulation, we estimated the lag error rate and fraction of in-phase templates per cycle following sequencing of a set of 41 DNA sequences derived from the PhiX genome using either a 55-cycle standard four nucleotide flow (control) or a flow order consisting of 10 cycles of four nucleotides followed by 70 cycles consisting of three randomly selected nucleotide types (equivalent to Random 3 order in FIG. 2). In this experiment, the use of an initial 10 cycle four nucleotide flow facilitates comparison of lead and lag error rates across the two experimental conditions. Each nucleotide contained a reversible terminator moiety and labeled by one of four fluorescent dyes, the signal of which could be quantified by four color channel image analysis. Sequencing conditions were selected to achieve a per cycle lag error rate of 1-2%. Approximately 8,000 template clusters from the control and test conditions were selected for downstream analysis. As expected, the net signal intensity per channel remained in a narrow range across all 55 cycles of the four-nucleotide control experiment (FIG. 7), while a reduction in signal of approximately one magnitude was observed in one of the four channels for each flow cycle employing a three-nucleotide flow within the test experiment (FIG. 8). In each of the latter cases, the channel showing a reduction in net signal intensity corresponded perfectly with the nucleotide absent from that cycle.

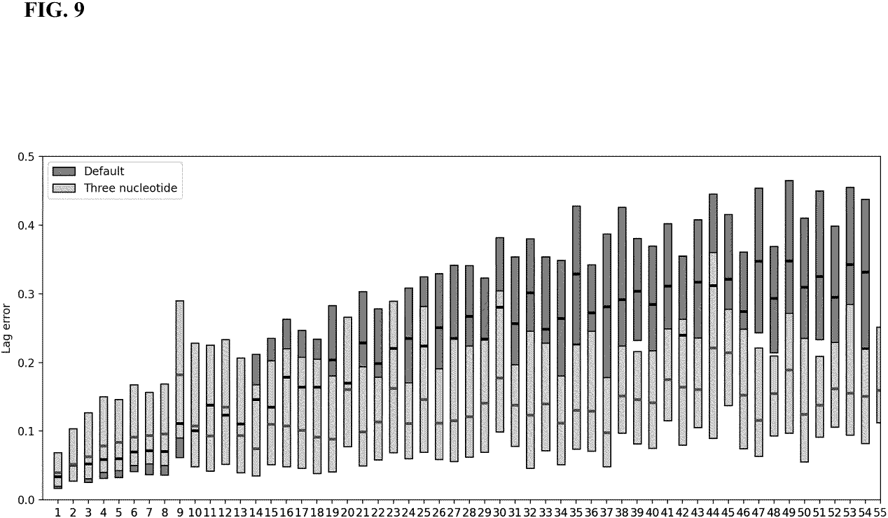

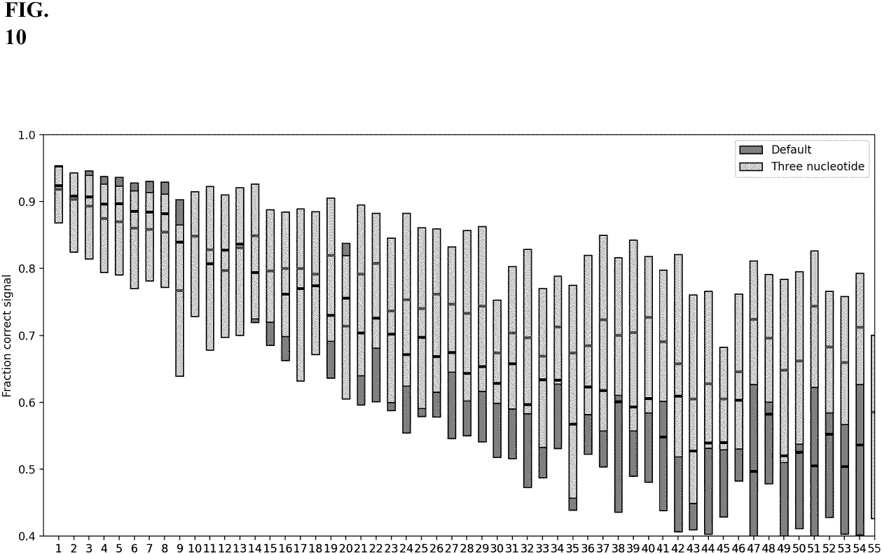

Given that the sequence of each of the 41 DNA sequences was predefined, the lag error rate and fraction of in-phase templates could be estimated by comparing the observed signal per channel for a given cycle with the expected signal based on the template sequence of each cluster. Accordingly, for each template cluster within the sequencing experiment, and each cycle within the experiment, the lag error rate was defined as the signal intensity of the channel corresponding to the previous nucleotide incorporation divided by the total signal across all four channels (FIG. 9), while the fraction of in-phase templates was defined as the ratio of the signal of the channel corresponding to the correct nucleotide divided by the total signal across all four channels (FIG. 10). If the previous nucleotide incorporation matched the incorporation in the current cycle, then the last nucleotide incorporation differing from the current nucleotide incorporation was used to select the signal channel corresponding to lag error. As seen in FIGS. 9 and 10, following cycle 10, the lag error of the three-nucleotide test condition diverges from the four-nucleotide control condition, with the three-nucleotide condition demonstrating an approximately 50% reduction in lag error compared to the control condition by nucleotide incorporation 55 and an approximately 20% increase in the fraction of in-phase molecules. These values are consistent with expectation based on the simulation results from Example 1 and highlight the superior phase protection of random flow orders compared to other orders, while also maintaining a relatively high sequencing efficiency.

### Example 3. Phase Protecting B-Cell and T-Cell Receptor Repertoire Sequencing

The functions of immune cells such as B- and T-cells are predicated on the recognition through specialized receptors of specific targets (antigens) in pathogens. There are approximately 10¹⁰-10¹¹B-cells and 10¹¹T-cells in a human adult (Ganusov VV, De Boer RJ. Trends Immunol. 2007;28(12):514-8; and Bains I, Antia R, Callard R, Yates AJ. Blood. 2009;113(22):5480-5487).

Immune cells are critical components of adaptive immunity and directly bind to pathogens through antigen-binding regions present on the cells. Within lymphoid organs (e.g., bone marrow for B cells and the thymus for T cells) the gene segments variable (V), joining (J), and diversity (D) rearrange to produce a novel amino acid sequence in the antigen-binding regions of antibodies that allow for the recognition of antigens from a range of pathogens (e.g., bacteria, viruses, parasites, and worms) as well as antigens arising from cancer cells. The large number of possible V-D-J segments, combined with additional (junctional) diversity, lead to a theoretical diversity of >10¹⁴, which is further increased during adaptive immune responses. Overall, the result is that each B- and T-cell expresses a practically unique receptor, whose sequence is the outcome of both germline and somatic diversity. These antibodies also contain a constant (C) region, which confers the isotype to the antibody. In most mammals, there are five antibody isotypes: IgA, IgD, IgE, IgG, and IgM. For example, each antibody in the IgA isotype shares the same constant region.

While parts of the B-cell immunoglobulin receptor (BCR) can be traced back to segments encoded in the germline (i.e., the V, D and J segments), the set of segments used by each receptor is something that needs to be determined de novo as it is coded in a highly repetitive region of the genome (Yaari G, Kleinstein SH. Practical guidelines for B-cell receptor repertoire sequencing analysis. Genome Med. 2015;7:121. (2015)). Additionally, there are no pre-existing full-length templates to align the sequencing reads. Thus, obtaining long-range sequence data is incredibly insightful to gain insights into the adaptive immune response in healthy individuals and in those with a wide range of diseases.

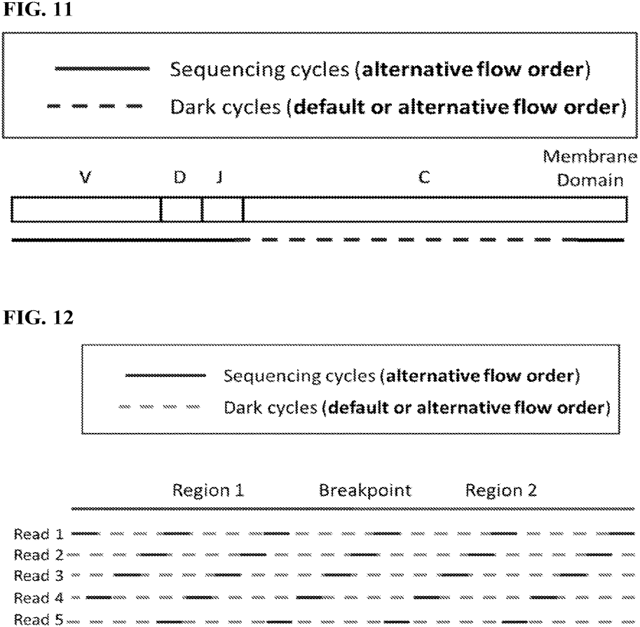

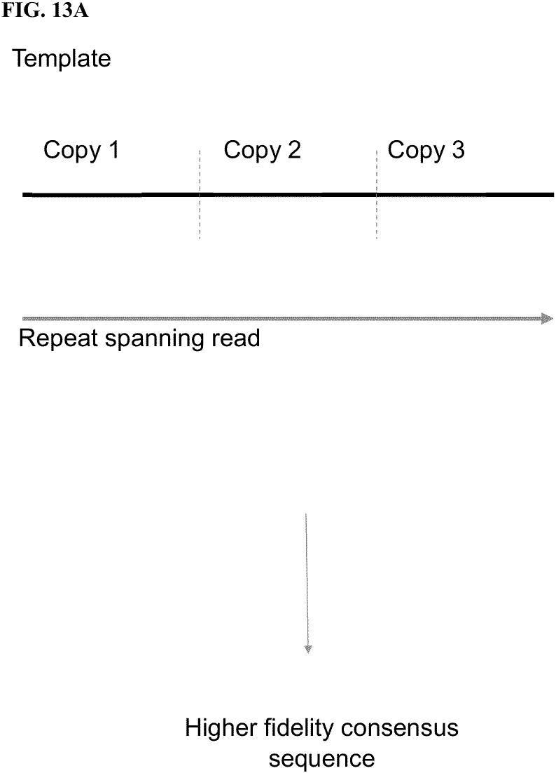

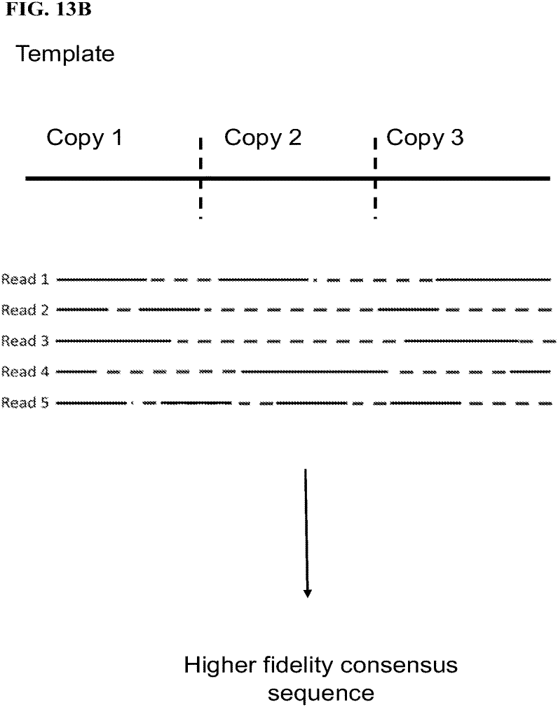

Described herein is a method for obtaining comprehensive snapshots of the repertoire diversity for each class of antibody by sequencing a portion of the constant region sufficient to determine the isotype and/or to determine whether a transmembrane domain is present, whereby the transmembrane domain is indicative of a surface bound receptor or secreted immunoglobulin, the method including a plurality of sequencing-cycles and a plurality of dark cycles (see, e.g., U.S. Pat. Appl. No. 17/127,308, which is incorporated by reference herein in its entirety), while taking advantage of the optimized random nucleotide flow orderings as described supra for increased accuracy and read length. The method further includes applying multiple dark cycles coupled with a standard four-nucleotide flow order to rapidly extend the elongating strand to the joining gene, then applying sequencing cycles with an alternative flow order, for example, a random AB flow, to obtain a comprehensive readout of the V-D-J segments, which determine the antigen specificity of the surface bound receptor or secreted immunoglobulin (see FIG. 11). In embodiments, the method involves alternating dark and sequencing cycles, in tandem with alternating flow orders, to obtain a set of reads that may be combined to precisely reconstruct a breakpoint region within a cancer cell (see FIG. 12). In other embodiments, the method involves applying a single long read to sequence tandemly arranged copies of a DNA region of interest (FIG. 13A). The resultant copy sequences may be compared to one another to detect and eliminate PCR and sequencing derived errors, and ultimately combined to form a higher quality consensus sequence. In yet further embodiments, sequencing of tandem copies of a DNA region of interest is accomplished by combining a set of reads derived from application of light and dark sequencing cycles (FIG. 13B).

In some embodiments, the dark cycle includes extending the complementary polynucleotide by one or more nucleotides using a polymerase; where the extension is accomplished by a pool of native nucleotides lacking at least one of the four bases. For example, the dark cycle may include extending the complementary nucleotide in the presence of three nucleotides, e.g., dA, dG, and dC. The cycles of extension may continue until the complement of the missing nucleotide, e.g., dT, is necessary to continue extension.

Following a plurality of dark cycles, a sequencing cycle, or a plurality of sequencing cycles, may be reinstated, whereby the extension strand from the limited-extension cycle (i.e., the dark-extension strand) is elongated in the presence of a polymerase and labeled nucleotide analogues. The sequence data is collected from a portion of the template nucleic acid sequence which is contiguous with the dark-extension strand, but not contiguous with the sequenced-extension strand from the first nucleic acid sequencing reaction.

The methods described herein permit faster sequencing of nucleic acid sequences with greater sequencing depth. In embodiments, the methods described herein are about or more than about 2-fold or 4-fold faster than traditional sequencing methodologies. When combined with a distribution of nucleic acid fragments and the massive parallelization that next-generation sequencing technology affords, in embodiments, the methods described herein may increase the sequencing read length to 500-1000 base pairs of a region of a reference sequence. Additionally, the inclusion of random flow orders, or other flow orders containing consecutive flows of the same solution (e.g., Thue-Morse sequences) during sequencing cycles will lead to superior phase protection while also maintaining a relatively high sequencing efficiency, and will improve long-read accuracy.

Sample library preparation involves the isolation and amplification of the target nucleic acid fragments for sequencing. Briefly, B cells are separated from the starting tissue (e.g., anticoagulated whole blood containing B cells). There are two starting materials that can serve as the initial template to sequence immunoglobulin (Ig) repertoires—genomic DNA (gDNA) and mRNA. In the example above, RNA input would be used as splicing eliminates large introns within the rearranged receptor, resulting in a constant gene region sequence that directly flanks the rearranged V-D-J. RNA is converted to cDNA by reverse transcription; in some embodiments, RNA derived from B cells may be selectively converted to cDNA using oligomers targeting the 3′ most region of the isotype. Optionally, IGH cDNA may be amplified by PCR, followed by NGS library preparation according to known techniques in the art, then subjected to alternating sequencing and dark cycles with random and traditional flow orders, respectively, (e.g., the interval sequencing protocols) as described herein and in further detail in U.S. Pat. Appl. No. 17/127,308, which is incorporated herein by reference.

### Example 4. Long Read Sequencing for Detection of Structural Variation in Cancer

Gene fusions and other structural variants are an important clinically actionable cancer biomarkers that may be detected by NGS sequencing of cancer DNA. Identification of structural variants may be facilitated by long sequencing reads. FIG. 12 provides an example of an implementation where sequencing via alternative flow orders is combined with interval sequencing to rapidly produce long breakpoint spanning reads.

### Example 5. Phase Protection During Enzymatic Polynucleotide Synthesis

At present, the majority of artificially synthesized oligonucleotides are created by chemical synthesis using the phosphoramidite process. Polynucleotides are also be synthesized enzymatically with a template-independent deoxyribonucleic acid (DNA) polymerase such as terminal deoxynucleotidyl transferase (TdT). Phosphoramidite synthesis is carried out by stepwise addition of nucleotide residues to the 5′-terminus of a growing polynucleotide until the desired sequence is assembled. Phosphoramidite synthesis involves a complex series of chemical reactions to join nucleoside phosphoramidites and creates organic waste that can be hazardous and expensive to process. Additionally, the upper limit of phosphoramidite-based oligo synthesis is about 200-300 nucleotides, and as a result, longer molecules must be assembled from oligonucleotides in failure-prone processes (see, Palluk S et al. Nat. Biotechnol. 2018; 36(7):645-650, which is incorporated herein by reference in its entirety). Enzymes used for enzymatic synthesis, such as TdT, can repeatedly add any available nucleotide in an unregulated manner. Multiple techniques have been developed to regulate the activity of template-independent polymerases. However, it can still be difficult to add only a single nucleotide at a time.

An alternative approach for synthesizing polynucleotides has been recently described (for details, see U.S. Pat. Pub. 2021/0340615, which is incorporated herein by reference in its entirety). This approach uses a universal template strand that includes universal base analogs that pair with any of the natural nucleotide bases. Primer extension with a polymerase is used to synthesize a polynucleotide with a de novo sequence that is complementary to the universal template strand. The universal template strand creates a double-stranded molecule with the growing polynucleotide. In some implementations, the universal template strand may have a backbone structure that is different from conventional DNA or ribonucleic acid (RNA).

Because the universal template strand can hybridize to any sequence, the sequence of the polynucleotide hybridized to the universal template strand is specified not by base pairing with the template strand but by the order in which protected nucleotides are added. Protected nucleotides include blocking groups that limit addition to only one nucleotide at a time. After a protected nucleotide is incorporated into a growing polynucleotide by a polymerase, the blocking group is removed and the next protected nucleotide is added. Multiple cycles of protected nucleotide addition and deblocking are repeated until synthesis of the polynucleotide is complete. The polynucleotide may be dehybridized from the universal template strand and stored or processed. The universal template strand may then be reused to create a different polynucleotide.

Multiple polynucleotides with different sequences can be created in parallel by anchoring universal template strands to a solid substrate and selectively deblocking protected nucleotides at only specific locations on the surface of the solid substrate. Location-specific deblocking may be achieved by any number of techniques that cause cleavage of blocking groups at some but not all of the nucleotides attached to the solid substrate. Techniques for controlling the locations at which blocking groups are removed include using a microelectrode array to vary electrical current, a photomask to control exposure to light, and inkjet printing to deposit chemicals at precise locations. Different combinations of locations on the surface of the solid substrate may be deblocked at each cycle which changes where protected nucleotides are added. Performing multiple cycles of addition in which the location of nucleotide addition and the base of the nucleotide are varied each cycle creates a high degree of parallelism and enables synthesis of a batch of polynucleotides with different sequences.

As described in Example 1 with respect to sequencing a polynucleotide template, at the start of a sequencing reaction, after hybridization of the sequencing primer, 100% of the strands within the cluster are synchronized. As the strands are extended, individual strands may fall behind or extend faster than the majority of the strands in the polynucleotide cluster, referred to as dephasing. Some strands may extend faster when the reversible terminator of the nucleotide to be incorporated is removed prematurely, or the solution of reversibly terminated nucleotides contains impurities (e.g., natural nucleotides or modified nucleotides bearing a 3′ hydroxyl group), resulting in the clusters of monoclonal amplicons being out-of-phase.

As in sequencing, dephasing may also arise during de novo enzymatic polynucleotide synthesis using a universal template as described herein. Each nucleotide incorporation associated with the population of universal templates may be described as being generally “in-phase” or having “phasic synchrony” with each other when they are performing the same incorporation step at the same sequence position for the associated universal template molecules in a given reaction step. A relatively small fraction of template molecules in each population may lose or fall out of phasic synchrony (e.g., may become “out of phase”) with the majority of the template molecules in the population. That is, the incorporation events associated with a certain fraction of template molecules may either get ahead of or fall behind other similar template molecules during polynucleotide synthesis.

Flow order methods as described supra in Examples 1 and 2 may be utilized during de novo enzymatic polynucleotide synthesis to reduce and/or correct the effects of dephasing. These flow order methods are useful for the enzymatic synthesis of polynucleotides from template strands where at least a subset of bases are not universal base analogs. If the template consists entirely of universal base analogs then the flow order will not impact phasing, as any introduced base will incorporate. By contrast, if the template consists of at least some non-universal nucleotides, synthesizing the complementary polynucleotide via a rephasing flow order will synchronize the synthesizing molecules, resulting in a greater fraction of synthesized molecules of the same length. For example, application of restricted flow orders would improve performance when using a template consisting of two stretches of universal bases separated by a portion of non-universal bases. Thue-Morse sequences and random two solution flow orders may be applied to enzymatic polynucleotide synthesis to provide phase protection and improved polynucleotide synthesis efficiency. Random AB refers to a random selection between two reagents, reagent A which contains two nucleotides types (e.g., dA and dC); and reagent B includes two other nucleotides (e.g., dT and dG). As illustrated in Table 1 and FIG. 2, a default four nucleotide flow order provides no protection against dephasing, while non-predetermined flow orders, such as those defined by a random alternation between two nucleotide reagent solutions, afford superior phase protection compared to predetermined orders. Thue-Morse sequences are enriched for consecutive repetitions of the same item and have been shown to yield a balanced alternation between two items irrespective of sequence length (Richman, R. “Recursive Binary Sequences of Differences” (2001). Complex Systems. 13 (4):381-382 ). As shown in FIG. 5 and Table 1, Thue-Morse sequences (Thue-Morse, Thue-Morse 2, Thue-Morse 3, and Thue-Morse 4, as shown in Table 2) outperformed the Random AB flow order with respect to phase protection and overall base quality during sequencing. Implementing these two-solution flow orders into enzymatic polynucleotide synthesis protocols as described herein may also yield similar improvements in phase protection and synthesis efficiency.

### EMBODIMENTS

The present disclosure provides the following illustrative embodiments.

Embodiment P1. A method for sequencing a nucleic acid template, said method comprising: a) hybridizing one or more sequencing primers to a nucleic acid template; b) executing a plurality of sequencing cycles, each cycle comprising (i) contacting the nucleic acid template with a sequencing solution in the presence of a polymerase, wherein the sequencing solutions of at least two sequencing cycles comprise a different combination of fewer than four nucleotide types, wherein each nucleotide of each nucleotide type comprises a reversible terminator; and (ii) detecting a characteristic signature indicating that the nucleotide has been incorporated into the sequencing primer.

Embodiment P2. The method of Embodiment P1, wherein the method comprises executing a plurality of sequencing cycles by consecutively contacting the nucleic acid template with a first sequencing solution, followed by consecutively contacting the nucleic acid template with a second sequencing solution, wherein the first sequencing solution is different than the second sequencing solution.

Embodiment P3. The method of Embodiment P2, wherein consecutively contacting comprises contacting the nucleic acid template 2 to 10 times with a first or second sequencing solution.

Embodiment P4. The method of Embodiment P2, wherein consecutively contacting comprises contacting the nucleic acid template 2 to 4 times with a first or second sequencing solution.

Embodiment P5. The method of Embodiment P2, wherein consecutively contacting comprises contacting the nucleic acid template 2 times with a first or second sequencing solution.

Embodiment P6. The method of any one of Embodiment P1 to Embodiment P5, wherein each of a plurality of the sequencing solutions comprises a different combination of two nucleotide types.

Embodiment P7. The method of any one of Embodiment P1 to Embodiment P5, wherein each of a plurality of the sequencing solutions comprises a different combination of three nucleotide types.

Embodiment P8. The method of any one of Embodiment P1 to Embodiment P5, wherein each of a plurality of the sequencing solutions comprises a randomly determined combination of less than four nucleotide types.

Embodiment P9. The method of any one of Embodiment P1 to Embodiment P5, wherein each of a plurality of the sequencing solutions comprises a randomly determined combination of three nucleotide types.

Embodiment P10. The method of any one of Embodiment P1 to Embodiment P5, wherein each of a plurality of the sequencing solutions comprises a randomly determined combination of two nucleotide types.

Embodiment P11. The method of any one of Embodiment P1 to Embodiment P10, wherein greater than 10%, 20%, 30%, 40%, or 50% of the plurality of sequencing cycles comprise a sequencing solution that comprises a plurality of less than four nucleotide types.

Embodiment P12. The method of any one of Embodiment P1 to Embodiment P10, wherein greater than 50%, 60%, 70%, 80%, 90%, 95%, or 99% of the plurality of sequencing cycles comprise a sequencing solution that comprises a plurality of less than four nucleotide types.

Embodiment P13. The method of any one of Embodiment P1 to Embodiment P10, wherein about 50%, 60%, 70%, 80%, 90%, 95%, or 99% of the plurality of sequencing cycles comprise a sequencing solution that comprises a plurality of less than four nucleotide types.

Embodiment P14. The method of any one of Embodiment P1 to Embodiment P13, wherein prior to detecting the characteristic signature, the method further comprises contacting the nucleic acid templates with a dark solution, wherein the dark solution comprises a plurality of unlabeled, 3′-reversibly terminated dATPs, dCTPs, dTTPs, dGTPs.

Embodiment P15. A method for sequencing a nucleic acid template, said method comprising: a) hybridizing one or more sequencing primers to a nucleic acid template; b) executing a plurality of sequencing cycles, each cycle comprising (i) contacting the nucleic acid template with a sequencing solution in the presence of a polymerase, wherein the sequencing solutions of at least two sequencing cycles comprise a different combination of a plurality of four different nucleotide types, wherein at least one nucleotide type is a non-incorporating nucleotide type, and the remaining nucleotide types each comprise a reversible terminator; and (ii) detecting a characteristic signature indicating that the nucleotide has been incorporated into the sequencing primer.

Embodiment P16. The method of Embodiment P15, wherein the method comprises executing a plurality of sequencing cycles by consecutively contacting the nucleic acid template with a first sequencing solution, followed by consecutively contacting the nucleic acid template with a second sequencing solution, wherein the first sequencing solution is different than the second sequencing solution.

Embodiment P17. The method of Embodiment P16, wherein consecutively contacting comprises contacting the nucleic acid template 2 to 10 times with a first or second sequencing solution.

Embodiment P18. The method of Embodiment P16, wherein consecutively contacting comprises contacting the nucleic acid template 2 to 4 times with a first or second sequencing solution.

Embodiment P19. The method of Embodiment P16, wherein consecutively contacting comprises contacting the nucleic acid template 2 times with a first or second sequencing solution.

Embodiment P20. The method of any one of Embodiment P15 to Embodiment P19, wherein the sequencing solutions comprise a first nucleotide type comprising a reversible terminator and a second nucleotide type comprising a reversible terminator, and two non-incorporating nucleotide types.

Embodiment P21. The method of any one of Embodiment P15 to Embodiment P19, wherein the sequencing solutions comprise a first nucleotide type comprising a reversible terminator, a second nucleotide type comprising a reversible terminator, and a third nucleotide type comprising a reversible terminator, and one non-incorporating nucleotide type.

Embodiment P22. The method of any one of Embodiment P15 to Embodiment P19, wherein the sequencing solutions comprise a first nucleotide type comprising a reversible terminator and three non-incorporating nucleotide types.

Embodiment P23. The method of any one of Embodiment P15 to Embodiment P22, wherein greater than 10%, 20%, 30%, 40%, or 50% of the plurality of sequencing cycles comprise a sequencing solution that comprises a plurality of a first nucleotide type comprising a reversible terminator and a second nucleotide type comprising a reversible terminator, and two non-incorporating nucleotide types.

Embodiment P24. The method of any one of Embodiment P15 to Embodiment P22, wherein greater than 50%, 60%, 70%, 80%, 90%, 95%, or 99% of the plurality of sequencing cycles comprise a sequencing solution that comprises a plurality of a first nucleotide type comprising a reversible terminator and a second nucleotide type comprising a reversible terminator, and two non-incorporating nucleotide types.

Embodiment P25. The method of any one of Embodiment P15 to Embodiment P22, wherein about 50%, 60%, 70%, 80%, 90%, 95%, or 99% of the plurality of sequencing cycles comprise a sequencing solution that comprises a plurality of a first nucleotide type comprising a reversible terminator and a second nucleotide type comprising a reversible terminator, and two non-incorporating nucleotide types.

Embodiment P26. The method of any one of Embodiment P1 to Embodiment P25, wherein detecting a characteristic signature comprises detecting the absence of a label.

Embodiment P27. The method of any one of Embodiment P1 to Embodiment P26, wherein detecting a characteristic signature comprises detecting a fluorescent emission.

Embodiment P28. The method of any one of Embodiment P1 to Embodiment P27, wherein the reversible terminator is a 3′-reversible terminator.

Embodiment P29. The method of any one of Embodiment P1 to Embodiment P27, wherein the reversible terminator is a virtual terminator.

Embodiment P30. The method of any one of Embodiment P1 to Embodiment P27, wherein each nucleotide comprises a 3′-reversible terminator and a detectable label.

Embodiment P31. The method of any one of Embodiment P15 to Embodiment P27, wherein each non-incorporating nucleotide comprises a 3′—OH.

Embodiment P32. The method of any one of Embodiment P1 to Embodiment P31, wherein the method comprises executing a plurality of sequencing cycles by contacting the nucleic acid template with a series of sequencing solutions according to a predetermined flow order.

Embodiment P33. The method of Embodiment P32, wherein the predetermined flow order comprises a non-cyclic binary or non-cyclic ternary sequence.

Embodiment P34. The method of Embodiment P32, wherein the predetermined flow order comprises a Thue-Morse sequence.

Embodiment P35. The method of any one of Embodiment P1 to Embodiment P31, wherein the method comprises executing a plurality of sequencing cycles by contacting the nucleic acid template with a series of sequencing solutions according to a pseudorandom sequence flow order.

Embodiment P36. The method of any one of Embodiment P1 to Embodiment P35, wherein the template nucleic acid is about 50 to about 1500 nucleotides in length.

Embodiment P37. The method of any one of Embodiment P1 to Embodiment P35, wherein the template nucleic acid is about 50 to about 500 nucleotides in length.

Embodiment P38. The method of any one of Embodiment P1 to Embodiment P35, wherein the template nucleic acid is greater than 100 nucleotides in length.

Embodiment P39. The method of any one of Embodiment P1 to Embodiment P38, wherein at least 10 to 200 nucleotides are incorporated into the sequencing primer.

Embodiment P40. The method of any one of Embodiment P1 to Embodiment P38, wherein about 100 to 1000 nucleotides are incorporated into the sequencing primer.

Embodiment P41. The method of any one of Embodiment P1 to Embodiment P38, wherein about 100 to 500 nucleotides are incorporated into the sequencing primer.

Embodiment P42. The method of any one of Embodiment P1 to Embodiment P38, wherein greater than 200 nucleotides are incorporated into the sequencing primer.

Embodiment P43. The method of any one of Embodiment P1 to Embodiment P42, wherein each sequencing cycle comprises a probability of an incorrect base call that is less than 1 in 100.

Embodiment P44. The method of any one of Embodiment P1 to Embodiment P42, wherein each sequencing cycle comprises a probability of an incorrect base call that is less than 1 in 1000.

Embodiment P45. The method of any one of Embodiment P1 to Embodiment P42, wherein each sequencing cycle comprises a probability of an incorrect base call that is less than 1 in 100 for about 200 to 1000 nucleotide incorporations.

Embodiment P46. The method of any one of Embodiment P1 to Embodiment P42, wherein each sequencing cycle comprises a probability of an incorrect base call that is less than 1 in 1000 for about 200 to 1000 nucleotide incorporations.

Embodiment P47. The method of any one of Embodiment P1 to Embodiment P42, wherein each sequencing cycle comprises a probability of an incorrect base call that is less than 1 in 100 for about 500 to 1000 nucleotide incorporations.

Embodiment P48. The method of any one of Embodiment P1 to Embodiment P42, wherein each sequencing cycle comprises a probability of an incorrect base call that is less than 1 in 1000 for about 500 to 1000 nucleotide incorporations.

Embodiment P49. A kit for determining the identity of a base in a target nucleic acid by sequencing-by-synthesis, the kit comprising (a) a first sequencing mixture of deoxyribonucleotide triphosphates (dNTPs) comprising: a first plurality of dNTPs comprising a first label; and a second plurality of dNTPs comprising a second label; and (b) a second sequencing mixture of deoxyribonucleotide triphosphates (dNTPs) comprising: a third plurality of dNTPs comprising the first label; and a fourth plurality of dNTPs comprising the second label; wherein each of the first, second, third, and fourth pluralities of dNTPs is selected from the group consisting of dATP, dTTP, dCTP, and dGTP, and are different from each other; and wherein the first label and the second label are different labels and are distinguishable.

Embodiment P50. A method of sequencing a nucleic acid template, said method comprising hybridizing one or more sequencing primers to a nucleic acid template; executing a plurality of sequencing cycles, each cycle comprising contacting the nucleic acid template with the first and second sequencing mixtures of the kit of Embodiment P49 in the presence of a polymerase; and detecting a characteristic signature indicating that a nucleotide from the first or second sequencing mixtures has been incorporated into the sequencing primer.

Embodiment P51. A kit for determining the identity of a base in a target nucleic acid by sequencing-by-synthesis, the kit comprising (a) a first sequencing mixture of deoxyribonucleotide triphosphates (dNTPs) comprising: a first plurality of dNTPs comprising a first label; and a second plurality of dNTPs comprising a second label; and (b) a second sequencing mixture of deoxyribonucleotide triphosphates (dNTPs) comprising: a third plurality of dNTPs comprising the first label; and a fourth plurality of dNTPs comprising the second label; and (c) a third sequencing mixture comprising non-incorporating dNTPs comprising: a first plurality of non-incorporating dNTPs; a second plurality of non-incorporating dNTPs; a third plurality of non-incorporating dNTPs; and a fourth plurality of non-incorporating dNTPs; wherein each of the first, second, third, and fourth pluralities of dNTPs is selected from the group consisting of dATP, dTTP, dCTP, and dGTP, and are different from each other; wherein the first label and the second label are different labels and are distinguishable; and wherein each of the first, second, third, and fourth pluralities of non-incorporating dNTPs is selected from the group consisting of a non-incorporable analog of dATP, a non-incorporable analog of dTTP, a non-incorporable analog of dCTP, and a non-incorporable analog of dGTP, and are different from each other.

Embodiment P52. A method of sequencing a nucleic acid template, said method comprising hybridizing one or more sequencing primers to a nucleic acid template; executing a plurality of sequencing cycles, each cycle comprising contacting the nucleic acid template with the first, second, and third sequencing mixtures of the kit of Embodiment P51 in the presence of a polymerase; and detecting a characteristic signature indicating that a nucleotide from the first or second sequencing mixtures has been incorporated into the sequencing primer.

Embodiment P53. A device for sequencing a nucleic acid template, comprising: i) a reaction vessel for receiving flows of different sequencing solutions, wherein each of a plurality of the sequencing solutions comprises a different combination of two to three nucleotide types, wherein each nucleotide of each nucleotide type comprises a reversible terminator; ii) a plurality of reservoirs that each contain a different nucleotide type; iii) flow paths from each reservoir to the reaction vessel; and iv) a fluidics controller that controls the flow from the reservoirs to the reaction vessel, wherein the fluidics controller is programmed to randomly provide flow from the plurality of reservoirs to the reaction vessel in order to form the sequencing solutions.

Embodiment P54. A device for sequencing a nucleic acid template, comprising: i) a reaction vessel for receiving flows of different sequencing solutions, wherein each of a plurality of the sequencing solutions comprises a different combination of two to three nucleotide types, wherein each nucleotide of each nucleotide type comprises a reversible terminator; ii) a first reservoir that contains a first sequencing solution and a second reservoir that contains a second sequencing solution, wherein the first and second sequencing solutions collectively comprise all four nucleotide types; iii) flow paths from each reservoir to the reaction vessel; and iv) a fluidics controller that controls the flow from the reservoirs to the reaction vessel, wherein the fluidics controller is programmed to randomly provide flow from each of the reservoirs to the reaction vessel.

Embodiment P55. A device for sequencing a nucleic acid template, comprising: i) a reaction vessel for receiving flows of different sequencing solutions, wherein each of a plurality of the sequencing solutions comprises a different combination of four nucleotide types, wherein at least one nucleotide type is a non-incorporating nucleotide type, and the remaining nucleotide types each include a reversible terminator; ii) a first reservoir that contains a first sequencing solution and a second reservoir that contains a second sequencing solution, wherein the first and second sequencing solutions collectively comprise all four nucleotide types; iii) flow paths from each reservoir to the reaction vessel; and iv) a fluidics controller that controls the flow from the reservoirs to the reaction vessel, wherein the fluidics controller is programmed to randomly provide flow from each of the reservoirs to the reaction vessel.

Embodiment P56. A system, comprising: at least one data processor; and at least one memory storing instructions, which when executed by the at least one data processor, result in operations comprising: obtaining a plurality of sequencing reads from a genomic sequencing device, wherein each sequencing read is generated by executing a plurality of sequencing cycles, each cycle comprising contacting the nucleic acid template with a sequencing solution in the presence of a polymerase, wherein each sequencing solution comprises four nucleotide types; and detecting a characteristic signature indicating that the nucleotide has been incorporated into the sequencing primer; and obtaining a plurality of sequencing reads from a genomic sequencing device, wherein each sequencing read is generated according to any one of Embodiment P1 to Embodiment P48.

Embodiment P57. A system, comprising: at least one data processor; and at least one memory storing instructions, which when executed by the at least one data processor, result in operations comprising: obtaining a plurality of sequencing reads from a genomic sequencing device, wherein each sequencing read is generated by executing a plurality of sequencing cycles, each cycle comprising contacting the nucleic acid template with a sequencing solution in the presence of a polymerase, wherein each sequencing solution comprises four nucleotide types, wherein at least one nucleotide type is a non-incorporating nucleotide type, and the remaining nucleotide types each include a reversible terminator; and detecting a characteristic signature indicating that the nucleotide has been incorporated into the sequencing primer; and obtaining a plurality of sequencing reads from a genomic sequencing device, wherein each sequencing read is generated according to any one of Embodiment P1 to Embodiment P48.

### ADDITIONAL EMBODIMENTS

The present disclosure provides the following additional illustrative embodiments.

Embodiment 1. A method for sequencing a nucleic acid template, said method comprising: a) hybridizing one or more sequencing primers to a nucleic acid template; b) executing a plurality of sequencing cycles, each cycle comprising: (i) contacting the nucleic acid template with a sequencing solution in the presence of a polymerase; and (ii) detecting a characteristic signature indicating that the nucleotide has been incorporated into the sequencing primer; wherein the sequencing solutions of at least two sequencing cycles comprise a different combination of fewer than four nucleotide types, wherein each nucleotide of each nucleotide type comprises a reversible terminator.

Embodiment 2. The method of Embodiment 1, wherein the method comprises executing a plurality of sequencing cycles by contacting the nucleic acid template with a first sequencing solution, followed by contacting the nucleic acid template with a second sequencing solution, wherein the first sequencing solution comprises a first doublet of nucleotide types and said second sequencing solution comprises a second doublet of nucleotide types, wherein said first doublet of nucleotide types have no nucleotide types in common with said second doublet of nucleotide types.

Embodiment 3. The method of Embodiment 1, wherein the method comprises executing a plurality of sequencing cycles by contacting the nucleic acid template with a first sequencing solution, followed by contacting the nucleic acid template with a second sequencing solution, wherein said first sequencing solution comprises a first triplet of nucleotide types and said second sequencing solution comprises a second triplet of nucleotide types, wherein said first triplet of nucleotide types has one or two nucleotide types in common with said second triplet of nucleotide types.

Embodiment 4. The method of Embodiment 1, wherein the method comprises executing a plurality of sequencing cycles by contacting the nucleic acid template with a first sequencing solution, followed by contacting the nucleic acid template with a second sequencing solution, wherein said first sequencing solution comprises a first doublet of nucleotide types and said second sequencing solution comprises a second triplet of nucleotide types, wherein said first doublet of nucleotide types has one or two nucleotide types in common with said second triplet of nucleotide types.

Embodiment 5. The method of Embodiment 1, wherein the method comprises executing a plurality of sequencing cycles by contacting the nucleic acid template with a first sequencing solution, followed by contacting the nucleic acid template with a second sequencing solution, wherein said first sequencing solution comprises a first triplet of nucleotide types and said second sequencing solution comprises a second doublet of nucleotide types, wherein said first triplet of nucleotide types has one or two nucleotide types in common with said second doublet of nucleotide types.

Embodiment 6. The method of Embodiment 1, wherein the method comprises executing a plurality of sequencing cycles by consecutively contacting the nucleic acid template with a first sequencing solution, followed by consecutively contacting the nucleic acid template with a second sequencing solution, wherein the first sequencing solution is different than the second sequencing solution.

Embodiment 7. The method of Embodiment 1, wherein each of the sequencing solutions comprises a randomly determined combination of less than four nucleotide types.

Embodiment 8. The method of Embodiment 1, wherein each of the sequencing solutions comprises a randomly determined combination of two nucleotide types.

Embodiment 9. The method of Embodiment 1, wherein prior to or concurrent with detecting the characteristic signature, the method further comprises contacting the nucleic acid templates with a dark solution, wherein the dark solution comprises a plurality of unlabeled, 3′-reversibly terminated dATPs, dCTPs, dTTPs, or dGTPs.

Embodiment 10. The method of Embodiment 1, wherein each sequencing cycle comprises removing the sequencing solution.

Embodiment 11. The method of Embodiment 1, wherein detecting a characteristic signature comprises detecting the presence or absence of a label.

Embodiment 12. The method of Embodiment 1, wherein each nucleotide comprises a 3′-reversible terminator and a detectable label.

Embodiment 13. The method of Embodiment 1, wherein each sequencing solution further includes one or more non-incorporating nucleotide type.

Embodiment 14. The method of Embodiment 1, wherein the method comprises executing a plurality of sequencing cycles by contacting the nucleic acid template with a series of sequencing solutions according to a predetermined non-cyclic binary or non-cyclic ternary sequence flow order.

Embodiment 15. A method for extending a primer hybridized to a nucleic acid template, said method comprising: (a) contacting the primer with a first extension solution in the presence of a polymerase; (b) contacting the primer with a second extension solution in the presence of a polymerase thereby extending said primer by a single nucleotide; wherein: (i) said first extension solution comprises a first doublet of nucleotide types and said second extension solution comprises a second doublet of nucleotide types, wherein said first doublet of nucleotide types have no nucleotide types in common with said second doublet of nucleotide types; (ii) said first extension solution comprises a first triplet of nucleotide types and said second extension solution comprises a second triplet of nucleotide types, wherein said first triplet of nucleotide types has one or two nucleotide types in common with said second triplet of nucleotide types; (iii) said first extension solution comprises a first doublet of nucleotide types and said second extension solution comprises a second triplet of nucleotide types, wherein said first doublet of nucleotide types has one or two nucleotide types in common with said second triplet of nucleotide types; or (iv) said first extension solution comprises a first triplet of nucleotide types and said second extension solution comprises a second doublet of nucleotide types, wherein said first triplet of nucleotide types has one or two nucleotide types in common with said second doublet of nucleotide types; and (c) repeating steps (a) and (b), wherein each repetition of steps (a) and (b) is a cycle, wherein each cycle is performed at least 20 times thereby performing a series of cycles, wherein each cycle is a first ordered cycle or a second ordered cycle, wherein said first ordered cycle contacts the primer with said first extension solution first and said second extension solution second, wherein said second ordered cycle contacts the primer with said second extension solution first and said first extension solution second, wherein said series of cycles is performed according to a non-cyclic sequence.

Embodiment 16. The method of Embodiment 15, wherein prior to step c), the method further comprises detecting a characteristic signature indicating that the one to three nucleotides have been incorporated into the primer.

Embodiment 17. The method of Embodiment 15 or 16, wherein prior to step b), the method further comprises detecting a characteristic signature indicating that the one to three nucleotides have been incorporated into the primer.

Embodiment 18. The method of any one of Embodiment 15 to 17, wherein said second extension solution comprises a different combination of nucleotide types than said first extension solution.

Embodiment 19. The method of any one of Embodiment 15 to 17, wherein said second extension solution comprises the same combination of nucleotide types as said first extension solution.

Embodiment 20. The method of any one of Embodiment 15 to 17, wherein said first extension solution comprises a first doublet of nucleotide types and said second extension solution comprises a second doublet of nucleotide types, wherein said first doublet of nucleotide types have no nucleotide types in common with said second doublet of nucleotide types.

Embodiment 21. The method of any one of Embodiment 15 to 17, wherein said first extension solution comprises a first triplet of nucleotide types and said second extension solution comprises a second triplet of nucleotide types, wherein said first triplet of nucleotide types has one or two nucleotide types in common with said second triplet of nucleotide types.

Embodiment 22. The method of any one of Embodiment 15 to 17, wherein said first extension solution comprises a first doublet of nucleotide types and said second extension solution comprises a second triplet of nucleotide types, wherein said first doublet of nucleotide types has one or two nucleotide types in common with said second triplet of nucleotide types.

Embodiment 23. The method of any one of Embodiment 15 to 17, wherein said first extension solution comprises a first triplet of nucleotide types and said second extension solution comprises a second doublet of nucleotide types, wherein said first triplet of nucleotide types has one or two nucleotide types in common with said second doublet of nucleotide types.

Embodiment 24. The method of any one of Embodiment 15 to 17, wherein said first extension solution comprises a first triplet of nucleotide types and said second extension solution comprises a second triplet of nucleotide types, wherein said first triplet of nucleotide types has one or two nucleotide types in common with said second triplet of nucleotide types.

Embodiment 25. The method of any one of Embodiment 15 to 17, wherein said first extension solution comprises a first doublet of nucleotide types and said second extension solution comprises a second triplet of nucleotide types, wherein said first doublet of nucleotide types has two nucleotide types in common with said second triplet of nucleotide types.

Embodiment 26. The method of any one of Embodiment 15 to 17, wherein said first extension solution comprises a first triplet of nucleotide types and said second extension solution comprises a second doublet of nucleotide types, wherein said first triplet of nucleotide types has two nucleotide types in common with said second doublet of nucleotide types.

Embodiment 27. The method of any one of Embodiment 15 to 26, wherein each cycle is performed at least 30 times, at least 40 times, or at least 50 times.

Embodiment 28. The method of any one of Embodiment 15 to 27, wherein the nucleotide types of the first extension solution and the nucleotide types of the second extension solution differ across one or more cycles.

Embodiment 29. The method of any one of Embodiment 15 to 27, wherein the nucleotide types of the first extension solution and the nucleotide types of the second extension solution are the same across one or more cycles.

Embodiment 30. The method of any one of Embodiment 15 to 29, wherein prior to detecting the characteristic signature, the method further comprises contacting the primer with a dark solution, wherein the dark solution comprises a plurality of unlabeled, 3′reversibly terminated dATPs, dCTPs, dTTPs, or dGTPs.

Embodiment 31. The method of any one of Embodiment 15 to 30, wherein the non-cyclic sequence comprises a non-cyclic binary or non-cyclic ternary sequence.

Embodiment 32. The method of any one of Embodiment 15 to 30, wherein the non-cyclic sequence comprises a Thue-Morse sequence.

Embodiment 33. The method of any one of Embodiment 15 to 30, wherein the non-cyclic sequence is a pseudorandom sequence.

Embodiment 34. The method of any one of Embodiment 15 to 33, wherein each nucleotide of each nucleotide type comprises a reversible terminator.

Embodiment 35. The method of any one of Embodiment 15 to 34, wherein detecting a characteristic signature comprises detecting the absence of a label.

Embodiment 36. The method of any one of Embodiment 15 to 35, wherein detecting a characteristic signature comprises detecting a fluorescent emission.

Embodiment 37. The method of any one of Embodiment 15 to 36, wherein the reversible terminator is a 3′-reversible terminator.

Embodiment 38. The method of any one of Embodiment 15 to 36, wherein each nucleotide comprises a 3′-reversible terminator and a detectable label.

Embodiment 39. The method of any one of Embodiment 15 to 33, wherein at least one nucleotide type of said first extension solution, said second extension solution, or both said first extension solution and said second extension solution is a non-incorporating nucleotide type.

Embodiment 40. The method of any one of Embodiment 15 to 33, wherein at least one nucleotide type of said first extension solution, said second extension solution, or both said first extension solution and said second extension solution is a non-incorporating nucleotide type and the remaining one or more nucleotide types comprise a reversible terminator.

Embodiment 41. The method of any one of Embodiment 15 to 33, wherein two nucleotide types of said first extension solution, said second extension solution, or both said first extension solution and said second extension solution are non-incorporating nucleotide types.

Embodiment 42. The method of any one of Embodiment 15 to 33, wherein greater than 10%, 20%, 30%, 40%, or 50% of the cycles comprise a first extension solution, a second extension solution, or both a first extension solution and a second extension solution that comprises at least one non-incorporating nucleotide type.

Embodiment 43. The method of any one of Embodiment 1 to 42, wherein detecting a characteristic signature comprises detecting the absence of a label.

Embodiment 44. The method of any one of Embodiment 1 to 42, wherein detecting a characteristic signature comprises detecting a fluorescent emission.

Embodiment 45. The method of any one of Embodiment 1 to 44, wherein the reversible terminator is a 3′-reversible terminator.

Embodiment 46. The method of any one of Embodiment 1 to 45, wherein the template nucleic acid is about 50 to about 1500 nucleotides in length.

Embodiment 47. The method of any one of Embodiment 1 to 45, wherein the template nucleic acid is about 50 to about 500 nucleotides in length.

Embodiment 48. The method of any one of Embodiment 1 to 45, wherein the template nucleic acid is greater than 100 nucleotides in length.

Embodiment 49. The method of any one of Embodiment 1 to 48, wherein at least 10 to at least 200 nucleotides are incorporated into the primer.

Embodiment 50. The method of any one of Embodiment 1 to 48, wherein about 100 to about 200 nucleotides are incorporated into the primer.

Embodiment 51. A kit for determining the identity of a base in a target nucleic acid by sequencing-by-synthesis, the kit comprising (a) a first mixture of deoxyribonucleotide triphosphates (dNTPs) comprising: a first plurality of dNTPs comprising a first label; and a second plurality of dNTPs comprising a second label; and (b) a second mixture of deoxyribonucleotide triphosphates (dNTPs) comprising: a third plurality of dNTPs comprising the first label; and a fourth plurality of dNTPs comprising the second label; wherein each of the first, second, third, and fourth pluralities of dNTPs is selected from the group consisting of dATP, dTTP, dCTP, and dGTP, and are different from each other; and wherein the first label and the second label are different labels and are distinguishable.

Embodiment 52. A method of sequencing a nucleic acid template, said method comprising: hybridizing one or more sequencing primers to a nucleic acid template; executing a plurality of sequencing cycles, each cycle comprising contacting the nucleic acid template with the first and second mixtures of the kit of Embodiment 51 in the presence of a polymerase; and detecting a characteristic signature indicating that a nucleotide from the first or second mixtures has been incorporated into the sequencing primer.

Embodiment 53. A kit for determining the identity of a base in a target nucleic acid by sequencing-by-synthesis, the kit comprising (a) a first mixture of deoxyribonucleotide triphosphates (dNTPs) comprising: a first plurality of dNTPs comprising a first label; a second plurality of dNTPs comprising a second label; and a third plurality of dNTPs comprising a third label; (b) a second mixture of deoxyribonucleotide triphosphates (dNTPs) comprising: the second plurality of dNTPs comprising the first label; the third plurality of dNTPs comprising the second label; and a fourth plurality of dNTPs comprising the third label; (c) a third mixture of dNTPs comprising: the fourth plurality of dNTPs comprising the first label; the first plurality of dNTPs comprising the second label; and the second plurality of dNTPs comprising the third label; and (d) a fourth mixture of dNTPs comprising: the third plurality of dNTPs comprising the first label; the fourth plurality of dNTPs comprising the second label; and the first plurality of dNTPs comprising the third label; wherein each of the first, second, third, and fourth pluralities of dNTPs is selected from the group consisting of dATP, dTTP, dCTP, and dGTP, and are different from each other; and wherein the first label, the second label, and the third label are different labels and are distinguishable.

Embodiment 54. A method of sequencing a nucleic acid template, said method comprising: hybridizing one or more sequencing primers to a nucleic acid template; executing a plurality of sequencing cycles, each cycle comprising contacting the nucleic acid template with any two mixtures of the first, second, third, or fourth mixtures of the kit of Embodiment 53 in the presence of a polymerase, wherein the two mixtures collectively include all four nucleotide types; and detecting a characteristic signature indicating that a nucleotide from the first, second, third, or fourth mixtures has been incorporated into the sequencing primer.

Embodiment 55. A kit for determining the identity of a base in a target nucleic acid by sequencing-by-synthesis, the kit comprising (a) a first mixture of deoxyribonucleotide triphosphates (dNTPs) comprising: a first plurality of dNTPs comprising a first label; and a second plurality of dNTPs comprising a second label; and (b) a second mixture of deoxyribonucleotide triphosphates (dNTPs) comprising: a third plurality of dNTPs comprising the first label; and a fourth plurality of dNTPs comprising the second label; and (c) a third mixture comprising non-incorporating dNTPs comprising: a first plurality of non-incorporating dNTPs; a second plurality of non-incorporating dNTPs; a third plurality of non-incorporating dNTPs; and a fourth plurality of non-incorporating dNTPs; wherein each of the first, second, third, and fourth pluralities of dNTPs is selected from the group consisting of dATP, dTTP, dCTP, and dGTP, and are different from each other; wherein the first label and the second label are different labels and are distinguishable; and wherein each of the first, second, third, and fourth pluralities of non-incorporating dNTPs is selected from the group consisting of a non-incorporable analog of dATP, a non-incorporable analog of dTTP, a non-incorporable analog of dCTP, and a non-incorporable analog of dGTP, and are different from each other.

Embodiment 56. A method of sequencing a nucleic acid template, said method comprising hybridizing one or more sequencing primers to a nucleic acid template; executing a plurality of sequencing cycles, each cycle comprising contacting the nucleic acid template with the first, second, and third mixtures of the kit of Embodiment 55 in the presence of a polymerase; and detecting a characteristic signature indicating that a nucleotide from the first or second mixtures has been incorporated into the sequencing primer.

Embodiment 57. A device for sequencing a nucleic acid template, comprising: i) a reaction vessel for receiving flows of different solutions, wherein each of a plurality of the solutions comprises a different combination of two to three nucleotide types, wherein each nucleotide of each nucleotide type comprises a reversible terminator; ii) a plurality of reservoirs that each contain a different nucleotide type; iii) flow paths from each reservoir to the reaction vessel; and iv) a fluidics controller that controls the flow from the reservoirs to the reaction vessel, wherein the fluidics controller is programmed to randomly provide flow from the plurality of reservoirs to the reaction vessel in order to form the solutions.

Embodiment 58. A device for sequencing a nucleic acid template, comprising: i) a reaction vessel for receiving flows of different solutions, wherein each of a plurality of the solutions comprises a different combination of two to three nucleotide types, wherein each nucleotide of each nucleotide type comprises a reversible terminator; ii) a first reservoir that contains a first solution and a second reservoir that contains a second solution, wherein the first and second solutions collectively comprise all four nucleotide types; iii) flow paths from each reservoir to the reaction vessel; and iv) a fluidics controller that controls the flow from the reservoirs to the reaction vessel, wherein the fluidics controller is programmed to randomly provide flow from each of the reservoirs to the reaction vessel.

Embodiment 59. A device for sequencing a nucleic acid template, comprising: i) a reaction vessel for receiving flows of different solutions, wherein each of a plurality of the solutions comprises a different combination of four nucleotide types, wherein at least one nucleotide type is a non-incorporating nucleotide type, and the remaining nucleotide types each include a reversible terminator; ii) a first reservoir that contains a first solution and a second reservoir that contains a second solution, wherein the first and second solutions collectively comprise all four nucleotide types; iii) flow paths from each reservoir to the reaction vessel; and iv) a fluidics controller that controls the flow from the reservoirs to the reaction vessel, wherein the fluidics controller is programmed to randomly provide flow from each of the reservoirs to the reaction vessel.

1. A method for sequencing a nucleic acid template, said method comprising:
a) hybridizing a sequencing primer to the nucleic acid template;
b) executing a plurality of sequencing cycles by contacting the nucleic acid template with a series of sequencing solutions according to a predetermined non-cyclic binary or non-cyclic ternary sequence flow order, wherein each cycle comprising:
(i) contacting the nucleic acid template with a sequencing solution in the presence of a polymerase, wherein sequencing solutions of at least two sequencing cycles comprise a different combination of fewer than four nucleotide types, wherein each nucleotide of each nucleotide type comprises a reversible terminator; and
(ii) detecting a characteristic signature indicating that the nucleotide has been incorporated into the sequencing primer.

2. The method of claim 1, wherein executing a plurality of sequencing cycles further comprises contacting the nucleic acid template with a first sequencing solution, followed by contacting the nucleic acid template with a second sequencing solution, wherein the first sequencing solution comprises a first doublet of nucleotide types and said second sequencing solution comprises a second doublet of nucleotide types, wherein said first doublet of nucleotide types have no nucleotide types in common with said second doublet of nucleotide types.

3. The method of claim 1, wherein executing a plurality of sequencing cycles further comprises contacting the nucleic acid template with a first sequencing solution, followed by contacting the nucleic acid template with a second sequencing solution, wherein said first sequencing solution comprises a first triplet of nucleotide types and said second sequencing solution comprises a second triplet of nucleotide types, wherein said first triplet of nucleotide types has one or two nucleotide types in common with said second triplet of nucleotide types.

4. The method of claim 1, wherein executing a plurality of sequencing cycles further comprises contacting the nucleic acid template with a first sequencing solution, followed by contacting the nucleic acid template with a second sequencing solution, wherein said first sequencing solution comprises a first doublet of nucleotide types and said second sequencing solution comprises a second triplet of nucleotide types, wherein said first doublet of nucleotide types has one or two nucleotide types in common with said second triplet of nucleotide types.

5. The method of claim 1, wherein executing a plurality of sequencing cycles further comprises contacting the nucleic acid template with a first sequencing solution, followed by contacting the nucleic acid template with a second sequencing solution, wherein said first sequencing solution comprises a first triplet of nucleotide types and said second sequencing solution comprises a second doublet of nucleotide types, wherein said first triplet of nucleotide types has one or two nucleotide types in common with said second doublet of nucleotide types.

6. The method of claim 1, wherein executing a plurality of sequencing cycles further comprises contacting the nucleic acid template with a first sequencing solution, followed by consecutively contacting the nucleic acid template with a second sequencing solution, wherein the first sequencing solution is different from the second sequencing solution.

7. The method of claim 1, wherein each of the sequencing solutions comprises a randomly determined combination of less than four nucleotide types.

8. The method of claim 1, wherein each of the sequencing solutions comprises a randomly determined combination of two nucleotide types.

9. (canceled)

10. (canceled)

11. (canceled)

12. The method of claim 1, wherein a subset of each nucleotide comprises a 3′-reversible terminator and a detectable label and a subset of nucleotides include one or more non-incorporating nucleotides lacking 3′-reversible terminator and a detectable label.

13. (canceled)

14. (canceled)

15. A method for extending a primer hybridized to a nucleic acid template, said method comprising:
(a) contacting the primer with a first extension solution in the presence of a polymerase;
(b) contacting the primer with a second extension solution in the presence of a polymerase thereby extending said primer by a single nucleotide; wherein:
(i) said first extension solution comprises a first doublet of nucleotide types and said second extension solution comprises a second doublet of nucleotide types, wherein said first doublet of nucleotide types have no nucleotide types in common with said second doublet of nucleotide types;
(ii) said first extension solution comprises a first triplet of nucleotide types and said second extension solution comprises a second triplet of nucleotide types, wherein said first triplet of nucleotide types has one or two nucleotide types in common with said second triplet of nucleotide types;
(iii) said first extension solution comprises a first doublet of nucleotide types and said second extension solution comprises a second triplet of nucleotide types, wherein said first doublet of nucleotide types has one or two nucleotide types in common with said second triplet of nucleotide types; or
(iv) said first extension solution comprises a first triplet of nucleotide types and said second extension solution comprises a second doublet of nucleotide types, wherein said first triplet of nucleotide types has one or two nucleotide types in common with said second doublet of nucleotide types; and
(c) repeating steps (a) and (b), wherein each repetition of steps (a) and (b) is a cycle, wherein each cycle is performed at least 20 times thereby performing a series of cycles, wherein each cycle is a first ordered cycle or a second ordered cycle, wherein said first ordered cycle contacts the primer with said first extension solution first and said second extension solution second, wherein said second ordered cycle contacts the primer with said second extension solution first and said first extension solution second, wherein said series of cycles is performed according to a non-cyclic sequence.

16. The method of claim 15, wherein prior to step b) or c), the method further comprises detecting a characteristic signature indicating that the one to three nucleotides have been incorporated into the primer.

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. The method of claim 15, wherein the nucleotide types of the first extension solution and the nucleotide types of the second extension solution differ across one or more cycles.

29. The method of claim 15, wherein the nucleotide types of the first extension solution and the nucleotide types of the second extension solution are the same across one or more cycles.

30. (canceled)

31. (canceled)

32. The method of claim 15, wherein the non-cyclic sequence comprises a Thue-Morse sequence.

33. (canceled)

34. (canceled)

35. (canceled)

36. (canceled)

37. (canceled)

38. (canceled)

39. (canceled)

40. The method of claim 15, wherein at least one nucleotide type of said first extension solution, said second extension solution, or both said first extension solution and said second extension solution is a non-incorporating nucleotide type and the remaining one or more nucleotide types comprise a 3′-reversible terminator and detectable label.

41. (canceled)

42. The method of claim 15, wherein greater than 10%, 20%, 30%, 40%, or 50% of the cycles comprise a first extension solution, a second extension solution, or both a first extension solution and a second extension solution that comprises at least one non-incorporating nucleotide type.

43. (canceled)

44. (canceled)

45. (canceled)

46. The method claim 1, wherein the template nucleic acid is about 50 to about 1500 nucleotides in length.

47. (canceled)

48. (canceled)

49. The method claim 1, wherein at least 10 to at least 200 nucleotides are incorporated into the primer.

50. (canceled)

51. (canceled)

52. (canceled)

53. (canceled)

54. (canceled)

55. A kit for determining the identity of a base in a target nucleic acid by sequencing-by-synthesis, the kit comprising (a) a first mixture of deoxyribonucleotide triphosphates (dNTPs) comprising: a first plurality of dNTPs comprising a first label; and a second plurality of dNTPs comprising a second label; and (b) a second mixture of deoxyribonucleotide triphosphates (dNTPs) comprising: a third plurality of dNTPs comprising the first label; and a fourth plurality of dNTPs comprising the second label; and (c) a third mixture comprising non-incorporating dNTPs comprising: a first plurality of non-incorporating dNTPs; a second plurality of non-incorporating dNTPs; a third plurality of non-incorporating dNTPs; and a fourth plurality of non-incorporating dNTPs; wherein each of the first, second, third, and fourth pluralities of dNTPs is selected from the group consisting of dATP, dTTP, dCTP, and dGTP, and are different from each other; wherein the first label and the second label are different labels and are distinguishable; and wherein each of the first, second, third, and fourth pluralities of non-incorporating dNTPs is selected from the group consisting of a non-incorporable analog of dATP, a non-incorporable analog of dTTP, a non-incorporable analog of dCTP, and a non-incorporable analog of dGTP, and are different from each other.

56. A method of sequencing a nucleic acid template, said method comprising hybridizing one or more sequencing primers to a nucleic acid template; executing a plurality of sequencing cycles, each cycle comprising contacting the nucleic acid template with the first, second, and third mixtures of the kit of claim 55 in the presence of a polymerase; and detecting a characteristic signature indicating that a nucleotide from the first or second mixtures has been incorporated into the sequencing primer.

57. (canceled)

58. (canceled)

59. (canceled)
